SEQUENCE LISTING (71 sequences)

SEQ ID NO: 1 - DNA, 3724 bp, Homo Sapiens
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcataccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaaaaaaaaaaaaaa

SEQ ID NO: 2 - DNA, 3724 bp, Homo sapiens
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcgaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaaaaaaaaaaaaaa

SEQ ID NO: 3 - DNA, 2949 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagacagcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 4 - DNA, 5312 bp, Homo sapiens
ggccgcggcggcggaggcagcagcggcggcggcaatggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 5 - DNA, 5601 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 6 - DNA, 3256 bp, Homo sapiens
aggatacagcggcttctgcgcgacttataagagctccttgtgcggcgccattttaagcctctcggtctgtggcagcagcgttggcccggccccgggagcggagagcgaggggaggcggagagggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattg

SEQ ID NO: 7 - DNA, 5572 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgctggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcaaaaaaaaaaaaaaaaaaaaaaaaaaa

SEQ ID NO: 8 - DNA, 74 bp, Homo sapiens
tgaaaagaattcgcggccgcataacttcgtataatgtatgctatacgaagttatgttttcatgctaagttcgat

SEQ ID NO: 9 - DNA, 35 bp, Homo sapiens
aaataagaattctgatttttgtgaatactgggaac

SEQ ID NO: 10 - DNA, 32 bp, Homo sapiens
tcacaatctagagtgttcttattttttatgta

SEQ ID NO: 11 - DNA, 31 bp, Homo sapiens
ctcacttctagaagcaggccagtcaactcct

SEQ ID NO: 12 - DNA, 41 bp, Homo sapiens
atatcagcggccgcagaattcgttgccattaagccagtctg

SEQ ID NO: 13 - DNA, 32 bp, Homo sapiens
gatatccaattcttttatttaaactattatac

SEQ ID NO: 14 - DNA, 40 bp, Homo sapiens
ataatatctagaactagttgttgtggtgaagaaaagagag

SEQ ID NO: 15 - DNA, 38 bp, Homo sapiens
gaatcttctagaactagttctgaggtggaatggtgtca

SEQ ID NO: 16 - DNA, 71 bp, Homo sapiens
ggaaatgaattcgcggccgcataacttcgtataatgtatgctatacgaagttatatcagtggtcctgtgag

SEQ ID NO: 17 - DNA, 33 bp, Homo sapiens
cccactgaattcagaaagggaaagacatagaaa

SEQ ID NO: 18 - DNA, 34 bp, Homo sapiens
ctttccgctagcagctctagtgggtataactccc

SEQ ID NO: 19 - DNA, 34 bp, Homo sapiens; identical to SEQ ID NO: 18

SEQ ID NO: 20 - DNA, 40 bp, Homo sapiens
taggcggaattcgcggccgccggctcacttgcatctctta

SEQ ID NO: 21 - DNA, 34 bp, Homo sapiens
tgactggaattctgtatcgtaatgactgtacttc

SEQ ID NO: 22 - DNA, 32 bp, Homo sapiens
cattactctagacgtctgcagtcaactggaat

SEQ ID NO: 23 - DNA, 68 bp, Homo sapiens
gacagttctagaataacttcgtatagcatacattatacgaagttatatatcctcatctgcttgggatg

SEQ ID NO: 24 - DNA, 40 bp, Homo sapiens
ttatttgatatcgcggccgcaggcttgcagtgttttctcc

SEQ ID NO: 25 - DNA, 32 bp, Homo sapiens
ctggatgatatcatgatttacagaaaaagcaa

SEQ ID NO: 26 - DNA, 32 bp, Homo sapiens
tctgtaactagtctgtgaatccagaggggaaa

SEQ ID NO: 27 - DNA, 32 bp, Homo sapiens
gcacagactagttggcaaagaacacaaaagga

SEQ ID NO: 28 - DNA, 40 bp, Homo sapiens
ggtttcgaattcgcggccgcgctyggtcttgaactcccaa

SEQ ID NO: 29 - DNA, 36 bp, Homo sapiens
ttggaggaattcatgttaataccttcaggtctttgc

SEQ ID NO: 30 - DNA, 32 bp, Homo sapiens
aggtattctagacatttgctccaaactgacca

SEQ ID NO: 31 - DNA, 66 bp, Homo sapiens
tgtccatctagaataacttcgtataatgtatgctatacgaagttatgtgactgcttccaaaactgc

SEQ ID NO: 32 - DNA, 26 bp, Homo sapiens
tgttttcctttacttactacacctca

SEQ ID NO: 33 - DNA, 21 bp, Homo sapiens
cctgaaatttgtctgcgaagt

SEQ ID NO: 34 - DNA, 20 bp, Homo sapiens
tctttccgcctcagaaggta

SEQ ID NO: 35 - DNA, 21 bp, Homo sapiens
cctgaaatttgtctgcgaagt

SEQ ID NO: 36 - DNA, 23 bp, Homo sapiens
tgatttttgtgaatactgggaac

SEQ ID NO: 37 - DNA, 20 bp, Homo sapiens
gctgaggtgacccttgtctc

SEQ ID NO: 38 - DNA, 20 bp, Homo sapiens
gcttggctggacgtaaactc

SEQ ID NO: 39 - DNA, 20 bp, Homo sapiens
gcttggctggacgtaaactc

SEQ ID NO: 40 - DNA, 20 bp, Homo sapiens
ccacacagcaaagcagaaac

SEQ ID NO: 41 - DNA, 20 bp, Homo sapiens
gctggtaacatccacccaga

SEQ ID NO: 42 - DNA, 20 bp, Homo sapiens
gccttctatcgccttcttga

SEQ ID NO: 43 - DNA, 20 bp, Homo sapiens
acaaggacagttggggaatg

SEQ ID NO: 44 - DNA, 21 bp, Homo sapiens
tcttgacgagttcttctgagc

SEQ ID NO: 45 - DNA, 20 bp, Homo sapiens
aacaaggacagttggggaat

SEQ ID NO: 46 - DNA, 20 bp, Homo sapiens
cgtctgcagtcaactggaat

SEQ ID NO: 47 - DNA, 20 bp, Homo sapiens
aacaaggacagttggggaat

SEQ ID NO: 48 - DNA, 27 bp, Homo sapiens
ggtggagtatttgatagtgtattaacc

SEQ ID NO: 49 - DNA, 20 bp, Homo sapiens
aggacatagcgttggctacc

SEQ ID NO: 50 - DNA, 20 bp, Homo sapiens
tgggtagaatttcggggata

SEQ ID NO: 51 - DNA, 20 bp, Homo sapiens
ctgtgaatccagaggggaaa

SEQ ID NO: 52 - DNA, 20 bp, Homo sapiens
tgggtagaatttcggggata

SEQ ID NO: 53 - DNA, 23 bp, Homo sapiens
gggaaaaatatgacaaagaaagc

SEQ ID NO: 54 - DNA, 21 bp, Homo sapiens
tcttgacgagttcttctgagc

SEQ ID NO: 55 - DNA, 19 bp, Homo sapiens
ttgtgggagcccagaattt

SEQ ID NO: 56 - DNA, 20 bp, Homo sapiens
catttgctccaaactgacca

SEQ ID NO: 57 - DNA, 19 bp, Homo sapiens
ttgtgggagcccagaattt

SEQ ID NO: 58 - DNA, 20 bp, Homo sapiens
gctgcagtccattgagcata

SEQ ID NO: 59 - DNA, 21 bp, Homo sapiens
tccaggaagaggaaaggaaaa

SEQ ID NO: 60 - DNA, 20 bp, Homo sapiens
gctgcagtccattgagcata

SEQ ID NO: 61 - DNA, 20 bp, Homo sapiens
gcttggctggacgtaaactc

SEQ ID NO: 62 - DNA, 20 bp, Homo sapiens
gctgcagtccattgagcata

SEQ ID NO: 63 - DNA, 21 bp, Homo sapiens
tccaggaagaggaaaggaaaa

SEQ ID NO: 64 - DNA, 20 bp, Homo sapiens
gctgcagtccattgagcata

SEQ ID NO: 65 - DNA, 20 bp, Homo sapiens
tctttccgcctcagaaggta

SEQ ID NO: 66 - DNA, 21 bp, Homo sapiens
tccaggaagaggaaaggaaaa

SEQ ID NO: 67 - DNA, 20 bp, Homo sapiens
gatggagctgtggttgaggt

SEQ ID NO: 68 - DNA, 23 bp, Homo sapiens
tcaaaactgcattctgactttca

SEQ ID NO: 69 - DNA, 20 bp, Homo sapiens
gatggagctgtggttgaggt

SEQ ID NO: 70 - DNA, 20 bp, Homo sapiens
tctttccgcctcagaaggta

SEQ ID NO: 71 - DNA, 20 bp, Homo sapiens
caggacttgggaggtatcca

(19) United States
(12) Patent Application Publication
(10) Pub. No.: US 2012/0115896 A1
(43) Pub. Date: May 10, 2012

(54) ISOGENIC HUMAN CELL LINES COMPRISING MUTATED CANCER ALLELES AND PROCESS USING THE CELL LINES

(76) Inventors: Alberto Bardelli, Torino (IT); Federica Di Nicolantonio, La Loggia (TO) (IT); Sabrina Arena, Savigliano (Cuneo) (IT)

(21) Appl. No.: 13/132,737

(22) PCT Filed: Dec. 3, 2008
(86) PCT No.: PCT/EP2008/010218

§ 371 Date: Jan. 23, 2012

(51) Int. Cl. A61K 31/4375; A61P 35/00; C12Q 1/04; A61K 31/405; C12N 5/10; A01K 67/027

(52) U.S. Cl. 514/291; 435/366; 800/21; 435/34; 514/420

(57) ABSTRACT

Isogenic human cell lines comprising at least one mutated cancer allele under the control of the cell line endogenous promoter, which corresponds to the wild-type cancer allele promoter are disclosed, as well as an in vitro process for determining sensitivity/resistance of a patient suffering from a tumor to a pharmacological agent comprising the following steps: a) identifying at least one mutated cancer allele in a tissue affected by a tumor of said patient; b) providing an isogenic human cell line representative of the tissue, wherein the cell line comprises at least the identified mutated cancer allele, which is under the control of the cell line endogenous promoter corresponding to the wild-type cancer allele promoter; c) putting in contact said cell line with the pharmacological agent; d) determining a variation of proliferation, apoptosis or cytotoxicity of the cell line in presence of the pharmacological agent; wherein the variation of proliferation, apoptosis car cytotoxicity indicative of the sensitivity/resistance of the patient tumor to the pharmacological agent.

### TECHNICAL FIELD OF THE INVENTION

The present invention relates to human cell lines where selected oncogenes are inserted through a Knock In (KI) strategy. The present invention concerns also the use of these human cell lines as models for the detection of genotype-specific drug resistance.

### BACKGROUND OF THE INVENTION

The awareness that the discovery of cancer alleles can point to the identification of ‘druggable’ oncogenic pathways has led to a race to map the entire cancer genome. This initial imperative, supported by dramatic improvements in ‘Omics’ technologies and the availability of the reference human genome sequence, has fostered the identification of a large number of cancer-associated alleles. However, compared to the genomic discovery stage, the functional validation of putative novel cancer alleles—despite their potential clinical relevance—has substantially lagged behind. One of the current major goals in the field is now the clinical translation of this knowledge. In particular, there is an urgent need to evaluate how the presence or absence of one or more oncogenic alleles affects resistance/sensitivity to specifically targeted drugs. This is necessary for faster drug approval and to develop tailored therapy for those cancer patients who are resistant to pharmacological treatments.

The construction of model systems that accurately recapitulate the genetic alterations present in human cancer is a prerequisite to understand the cellular properties imparted by the mutated alleles and to identify genotype and tumor-specific pharmacological responses. In this regard, mammalian cell lines have been widely used as model systems to functionally characterize cancer alleles carrying point mutations or deletions and to develop and validate anticancer drugs. These models typically involve the ectopic expression (by means of plasmid transfection or viral infection) of mutated cDNAs in human or mouse cells. The derivative cells are then used to assess the properties of individual cancer alleles with a variety of standardized assays. Although these studies have yielded remarkable results, they are typically hampered by at least two caveats. First, the expression is achieved by transient or stable transfection of cDNAs, resulting often in overexpression of the target allele at levels that do not recapitulate what occurs in human cancers. Second, the expression of the mutated cDNA is achieved under the control of non-endogenous viral promoters. As a result, the mutated alleles cannot be appropriately (endogenously) modulated in the target cells. While such systems in which mutated oncogenes are ectopically expressed under exogenous promoters have been instrumental in dissecting their oncogenic properties, they have also led to controversial results. For example studies focused on oncogene-mediated transformation and senescence in mouse models have generated conflicting data depending on whether the cancer alleles were ectopically expressed or permanently introduced in the genome of mouse cells (1). Furthermore, it has been previously noted that transformation of mammalian cells by mutated human RAS cDNAs depends on at least 100-fold higher expression than is observed in human tumors (2).

Evidently, the above models, although extremely useful in determining the oncogenic potential of a single mutated allele, are limited by the fact that ectopic expression of such allele cannot completely reflect the gradual progression of a normal cell into a tumor one. Besides the risk of providing artifactual evidence, such approach might also limit the possibility to detect intermediate phases which may indeed reveal potential additional targets for drug therapy. Based on this reasoning, it should be clear that there is a urgent need for a reliable cellular model to be used to test anticancer drug efficacy and efficiency, that should not be carrying ectopic expression of mutated alleles. In order for such a model to be of use for cancer patients, it is indispensable that the impact on cell biology of the oncogenic mutations carried by the model closely reproduce cell progression toward a tumor phenotype in a human subject.

### SUMMARY OF THE INVENTION

Object of the present invention is the provision of a new model closely reproducing cell progression toward a tumor phenotype in a human subject and a process for determining drug resistance/sensitivity in a human subject suffering from a tumor.

According to the present invention said objects are achieved thanks to the solution having the characteristics referred to specifically in the ensuing claims. Thus the claims form integral part of the technical teaching herein provided in relation to the present invention.

To achieve these objects, thus to overcome the limitations of current models, the present inventors have used targeted homologous recombination to introduce a panel of cancer alleles in human cells which will be controlled by an endogenous promoter, corresponding to the one of the wild type allele.

In an embodiment, the present disclosure concerns human cell lines comprising at least one mutated cancer allele, wherein the mutated cancer allele is under the control of the cell line endogenous promoter which corresponds to the wild-type cancer allele promoter, wherein the at least one cancer allele is selected among BRAF, EGFR, PIK3CA, PTEN, CTNNB1, c-KIT, c-MET, EPHA3, Erbb2, AKT1, FGFR2, MSH6, ABL1, STAT1, STAT4, RET, AKT3, TEK, VAV3, ALK, LYN, NOTCH, IDH1, ROR1, FLT3, ALK, SRC, BCL9, RPS6KA2, PDPK1, NTRK3, NTRK2, AKT3, KDR, MKK4, FBWX7, MEK1, OBSCN, TECTA, MLL3, NRAS, HRAS, TP53, APC, Rb1, CDKN2A (p16), BRCA1, BRCA2, PTCH1, VHL, SMAD4, PER1, MEN1, NF1, NF2, ATM, PTPRD. The isogenic cell lines, thus, closely recapitulate the occurrence of somatic mutation in human cancers.

In an embodiment, the present disclosure concerns the use of such isogenic cell lines in a screening method to test which genotypes might be sensitive or resistant to antitumor agents. More specifically, the present disclosure provides a pharmacogenomic platform for the rational design of targeted therapies for cancer patients:

In an embodiment, the present disclosure concerns an in vitro or in vivo process for determining sensitivity/resistance of a patient suffering from a tumor to a pharmacological agent, comprising the following steps:

a) identifying at least one mutated cancer allele in a tissue affected by a tumor of the patient;

b) providing an isogenic human cell line representative of this tissue, wherein the cell line comprises at least the identified mutated cancer allele put under the control of the cell line endogenous promoter, which corresponds to the wild-type cancer allele promoter;

c) putting in contact the isogenic cell line with the pharmacological agent to be evaluated;

d) determining a variation of proliferation, cytotoxicity or apoptosis of the isogenic cell line in presence of the pharmacological agent;

wherein the variation of proliferation, cytotoxicity or apoptosis of the isogenic cell line induced by the presence of the pharmacological agent is indicative of the sensitivity/resistance of the patient tumor to the evaluated pharmacological agent.

In a still further embodiment wherein the sensitivity/resistance is evaluated as the relative variation of proliferation, apoptosis and/or cytotoxicity between the isogenic human cell line comprising the identified mutated cancer allele and the wild-type isogenic human cell line, i.e. the cell line free of the mutated cancer allele.

In a further embodiment, the present disclosure concerns a cell bank comprising a plurality of isogenic human cell lines, wherein these cell lines comprise at least one mutated cancer allele put under the control of the cell line endogenous promoter, which corresponds to the wild-type cancer allele promoter.

In a still further embodiment the present disclosure concerns the use of human isogenic cell lines comprising at least one mutated cancer allele, wherein the mutated cancer allele is under the control of the cell line endogenous promoter which corresponds to the wild-type cancer allele promoter, for generating xenografts apt to induce tumor growth in a non-human laboratory animal model and correspondingly for producing non-human transgenic laboratory animals susceptible to develop a tumor carrying the mutated cancer allele.

The findings obtained in knock-in (KI) models can be translated to cancer cells in which the corresponding mutations naturally occur. In addition to providing new insights into the molecular basis of cellular transformation, the present results indicate that (KI) cell models can be successfully used to evaluate how oncogenic alleles affect resistance/sensitivity to anticancer therapies.

### DESCRIPTION OF THE DRAWINGS

The present invention will now be described in detail in relation to some preferred embodiments by way of non limiting examples, referring to the annexed figures, in which:

FIG. 1. Targeted knock-in (KI) of cancer mutations in human cells.

Structure of AAV targeting constructs. AAV vectors carrying oncogenic alleles either in the 5′ (BRAF, EGFR, CTNNB1 and PTEN) or the 3′ arm (KRAS and PIK3CA) were used to introduce the indicated mutations in human cells by homologous recombination. P, SV40 promoter; Neo, geneticin-resistance gene; ITR, inverted terminal repeat; triangles, loxP sites. The nucleotide and aminoacid changes are indicated.

FIG. 2. Biochemical analysis of hTERT-HME1 KI cells carrying oncogenic alleles.

(A) After starvation, EGFR mutated clones (A and B) and parental (WT) cells were treated with EGF (50 ng/mL) for the indicated times. Lysates were immunoblotted with anti-phospho-EGFR (Tyr1068) and total anti-EGFR, and total protein amount was determined with anti-actin antibody. (B) Activation of PI3K in serum starved PIK3CA (H1047R) KI and WT cells was measured by anti-phosphoAKT antibody. Lysates were immunoblotted also with anti-total AKT, and total protein amount was determined with anti-actin antibody. (C) KRAS mutated clones (A and B) and parental WT cells were serum starved for 48 hours and lysed. Levels of GTP-RAS were assessed by pull down with the recombinant RAF-CRIB domain and immunoblotting with anti-Pan-Ras (Ab-3) antibody. Total lysates were also immunoblotted with anti-Pan-Ras and antiactin antibody. The colorectal cancer cell line HCT 116 carrying a mutated KRAS D13 allele served as control. The columns represent the result of the densitometric analysis of the dot images corresponding to the GTP-RAS normalized on total RAS of the indicated cell lines. The numbers are referred to the untreated WT cells that were given an arbitrary value of 1. (D) WT and BRAF KI cells were grown in growth factor deprived medium, and the corresponding lysates were immunoblotted with the phospho-p44/42 Map kinase (Thr202/Tyr204), total MAPK1/MAPK2 and antiactin antibodies. The columns represent the result of the densitometric analysis of the dot images corresponding to the phosphorylation status of MAPK normalized on total MAPK. The numbers are referred to the untreated WT cells that were given an arbitrary value of 1.

FIG. 3. Transforming potential of cells carrying oncogenic alleles.

(A) An anchorage-independent growth assay was performed on hTERT-HME1 cells carrying the indicated genotypes, while HCT 116 colorectal cancer cells were used as positive control. The same assay was performed on cells infected with lentiviral vectors expressing the G13D KRAS or V600E BRAF mutations. A lentiviral vector encoding for luciferase was employed as a negative control. Representative photographs were taken after 3 weeks. (B) The area occupied by colonies was analyzed with BD Pathway HT bioimager and counted with BD AttoVision 1.5 software. Columns indicate mean area of four fields and error bars represent SD.

FIG. 4. Effect of the EGFR tyrosine kinase inhibitor erlotinib on KI cells.

(3) The effect of erlotinib treatment on cellular proliferation was assessed for hTERT-HME1 (A), MCF10A (B) and hTERT RPE-1 (C) isogenic clones carrying the indicated mutations. The average cell number was measured by determining ATP content in three replicate wells. Results are normalized to growth of cells treated with DMSO and are represented as mean±SD of at least three independent experiments.

FIG. 5. ‘Pharmarray’ analysis of hTERT-HME1 cells carrying the indicated alleles.

(A) Heatmap of the pharmacogenomic data (Pharmarray). Each column represents the average of multiple isogenic clones of the indicated genotype. Each row displays the results of differential response to drugs of the KI compared to WT cells. Drugs that—at the indicated concentrations—preferentially inhibit the growth of mutated cells are highlighted by the black color, while white color indicates compounds to which KI cells are more resistant than the WT counterpart. Grey boxes indicate no significant differences in response between KI and parental cells. Overall clustering of all the compounds by Fuzzy-SOM and of all the genotypes by hierarchical clustering. (B-F) Individual clusters composed of drugs with similar genotype-specific activity: (B) EGFR sensitive; (C) EGFR-PIK3CA DKI sensitive; (D) BRAF sensitive; (E) EGFR resistant; (F) KRAS sensitive; (G) PIK3CA sensitive and (H) KRAS/BRAF resistant cluster.

FIG. 6. Knock-in of PIK3CA mutations sensitizes cells to everolimus.

(A) Antiproliferative effects of everolimus on hTERT-HME1 WT, PIK3CA KI cells grown in complete media. Results are normalized to treatment with DMSO and represent mean±SD of at least three independent observations. (B and C) Dose-response curve to everolimus of the indicated PIK3CA KI clones obtained in MCF10A (B) and SW48 (C) cell lines.

FIG. 7. Genetic alterations in the KRAS and PIK3CA pathways are determinant of tumor cells' response to everolimus.

(A) Antiproliferative effects of everolimus on cancer cell lines. The mutational status of KRAS BRAF, PIK3CA and PTEN are indicated. (B) Two independent clones of HCT 116 colorectal cancer cells—in which the KRAS D13 allele was genetically deleted by homologous recombination (HKh-2 and HKe-3)—were more sensitive to everolimus than either their parental cells or a clone in which the KRAS WT allele was knocked out, but the mutated allele was retained (HK2-6). (C) DLD-1-derived cell clones that are knock-out for the mutated KRAS D13 allele (two independent clones DKO-3 and DKO-4) were more sensitive to everolimus than either the corresponding parental cells or a clone retaining only the KRAS mutated allele (DKO-1). Results are expressed as percent of viability compared to cells treated with DMSO only ('control') and represent mean±SD of at least three independent observations. Abbreviations: ampl and loss indicate respectively increased PIK3CA gene copy number in NIH:OVCAR-3 and lack of Pten expression in U-87 MG and PC-3 cells.

FIG. 8. Concomitant genetic and pharmacologic targeting of KRAS and PIK3CA pathways in colorectal cancer cells.

(A and B). The response of HCT 116 and DLD-1 to the MEK inhibitor CI-1040 is shown to be modulated either by the pharmacological inhibition of the PIK3CA pathway using everolimus or by genetic deletion of the mutant PIK3CA alleles. (A) HCT 116 and (B) DLD-1 cancer cells retaining the PIK3CA mutant R1047 and K545 alleles, respectively, were less sensitive to the MEK inhibitor CI-1040 than their isogenic counterparts carrying WT PIK3CA. Addition of a single fixed concentration of everolimus (10⁻⁷ M) shifts to the left the dose-response curve of CI-1040 in PIK3CA mutant cells, resulting in IC₅₀ values similar to those achieved in PIK3CA WT clones. The experiment was performed four times with similar results. Results of a representative experiment are shown and are indicated as percent of viability of vehicle-only treated cells by the ATP assay (mean±SD).

FIG. 9. Anchorage-independent growth of MCF10A cells carrying cancer mutations.

A soft agar growth assay was performed on WT and KI cells carrying the indicated genotypes, while DLD-1 colorectal cancer cells were used as positive control. Pictures of a representative experiment are shown.

FIG. 10. Effect of the EGFR tyrosine kinase inhibitor gefitinib on KI cells.

The effect of gefitinib treatment for 96 hours on cellular proliferation was assessed for hTERT-HME1 (A) and hTERT RPE-1 (B) isogenic clones. The average cell number at each indicated drug concentration was measured by determining ATP content in three replicate wells. Results are normalized to cell growth treated with corresponding amounts of DMSO and are represented as mean±SD of at least three independent experiments.

FIG. 11. hTERT RPE1 cells carry a KRAS activating mutation.

(A) Electropherograms showing the WT and mutated (Gly12 insAla-Gly) KRAS alleles in hTERT RPE-1 cells. (B) Levels of GTP-Ras were assessed in hTERT RPE-1 cells by pull down with the recombinant RAF-CRIB domain and immunoblotting with anti-Pan-Ras (Ab-3) antibody. The colorectal cancer cell lines HCT-116 and DLD1 carrying a mutated KRASD13 allele were used as positive controls, while hTERT-HME1 cells represented negative control. Total lysates were also immunoblotted with anti-Pan-Ras and anti-actin antibody.

FIG. 12. Growth curves of mutated cells carrying oncogenic alleles

Cellular proliferation of hTERT-HME1 KI clones in 96-well plastic culture plates was assessed using media containing either EGF, insulin, hydrocortisone and 5% FBS. Average cell number at each time point was measured by determining ATP content in quadruplicate wells. Data are represented as mean±SD of three independent experiments (***p<0.001). RLUs indicate relative light units.

FIG. 13. Graphical visualization using GEDAS of the differential pharmacological responses of KI cells to drugs.

Compounds that preferentially inhibit the growth of mutated cells are highlighted by the black color, while white indicates compounds to which KI cells are more resistant than the WT counterpart. Grey boxes indicate no significant differences in response between KI and parental cells. The cell genotype, the drug names and the logarithmic concentration at which compounds were tested are indicated.

FIG. 14. Effect of everolimus of HCT 116 and DLD-1 colorectal cancer cells.

(A) After 4 days' treatment with everolimus, HCT-116 colorectal cancer cells that had the mutated 1047R allele of PIK3CA genetically deleted by homologous recombination (WT) displayed similar sensitivity as either their parental cells (WT/H1047R, in black) or a clone in which the PIK3CA WT (1047H) allele was knocked out, but the mutated 1047R allele was retained (−/H1047R). (B) DLD-1-derived cells that are knock-out for the mutated PIK3CA K545 allele were as sensitive to everolimus as either the corresponding parental cells or a clone retaining only the PIK3CA mutated allele.

FIG. 15. Biochemical effects of everolimus treatment in HCT116 and its derivative KRAS WT/-HKe-3 clone.

(A, D) After 30 minutes'treatment with everolimus 500 nM, HCT 116 parental cells and its derivative KRAS WT/-HKe-3 clone were lysed and immunoblotted with the anti-phospho-P70S6K, totalP70S6K, phospho-MAPK and total MAPK (B, C, E) The same lysates were used also for ELISA measurements of total AKT, phosphoAKT (Thr308), phosphoAKT (Ser473), total RpS6 and phosphoRpS6 levels. Numbers indicate the ratio of phosphorylated protein related to total protein levels and are normalized respect to the untreated (NT) HCT 116 cells.

FIG. 16. Biochemical effects of everolimus treatment in hTERT-HME1 WT and KI cells.

(A, E) After 30 minutes' treatment with everolimus 500 nM, cells of the indicated genotype were lysed and immunoblotted with the anti-phospho-P70S6K, totalP70S6K, phospho-MAPK and total MAPK antibodies (B, C, D) The same lysates were also used for ELISA measurements of total AKT, phosphoAKT (Thr308), phosphoAKT (Ser473), total RpS6 and phosphoRpS6 levels. Numbers indicate the ratio of phosphorylated protein related to total protein levels and are normalized respect to the untreated (NT) hTERT-HME1 WT cells. DKI, Double Knock-In cells carrying both PIK3CA H1047R and KRAS G13D alleles.

FIG. 17. Oncogenic KRAS confers resistance to everolimus.

Effect of everolimus (72 hours) on proliferation of HKe-3 (HCT116-derivative KRAS WT clone) (A) and ME-180 (B) cells infected with control or KRASG13D lentivirus.

FIG. 18. Effects of everolimus on cell cycle.

(A) CFSE-labelled cells were analyzed by flow cytometry at the indicated time-points (top panels). The maximum fluorescence intensity for all samples was recorded at day 0 (depicted in filled black). Decrease of fluorescence intensity is proportional to the number of cell divisions and was measured at day 2, 4 and 7 (indicated on top of the graph). hTERT-HME1 WT (A1, B1), PIK3CA E545K (A2, B2) and H1047R KI (A3, B3) cells showed a similar pattern of cell doublings in absence of treatment. Exposure to everolimus 500 nM for 7 days resulted in decreased cell proliferation rate in all genotypes, with the effect being particularly evident in PIK3CA H1047R, less pronounced in PIK3CA E545K and only minimal in WT cells. (B) Cells of the indicated genotype were incubated with everolimus 500 nM for 48 h, after which cell cycle was analyzed by flow cytometry. No increase of the subG1 apoptotic fraction of cells was observed upon treatment. Representative data from 3 independent experiments are shown.

FIG. 19. Effects of indomethacin on PIK3CA mutated cells.

(A) Cell viability of hTERT-HME1 WT and PIK3CA KI cells treated with indomethacin for 96 h, normalized to cells treated with vehicle, measured by the ATP assay. Data represent mean±SD of at least three independent experiments. Statistical analysis was performed comparing values of % cell viability for each KI clone versus WT cells calculated at the same drug concentration (***p<0.001, by Bonferroni's multiple comparison t test). (B-C) After 96 h drug treatment, cells were stained with Hoechst 33323 (depicted in gray as exemplified by dashed arrow), while apoptotic and dead cells were counterstained with propidium iodide (depicted in white as exemplified by black solid arrow). Effect of indomethacin 100 μM on WT (B) and PIK3CA KI cells (C). Cells were photographed with a 10× Lens at the BD™ Pathway HT bioimager. A field of a representative experiment is shown.

### DETAILED DESCRIPTION OF THE INVENTION

The present invention will now be described in detail in relation to some preferred embodiments by way of non limiting examples.

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

The headings provided herein are for convenience only and do not interpret the scope or meaning of the embodiments.

The construction of cellular models carrying cancer-associated genetic alterations is a prerequisite to dissect their role in tumor progression and to target their oncogenic properties. Until now, strategies to study cancer mutations in human cells have mainly involved ectopic expression of the corresponding mutated cDNA under the control of non-endogenous, constitutively active promoters These approaches do not accurately recapitulate the occurrence of cancer mutations in human tumors.

The above problem has found a solution in the present invention in that to overcome these limitations targeted homologous recombination to introduce cancer alleles in the genome of human cells by stable modification of the corresponding genomic locus was used. As a result, the heterozygously mutated genes were expressed under their endogenous promoters, thus closely recapitulating the lesions observed in human tumors. This type of technical solution possess a clear advantage over the precedent ones in that it provides a realistic cellular model of tumor evolution and biology, which can likely be transposed to human subject. Target cells for the introduction (knock-in) or deletion (knock-out) of cancer alleles include those that are able to or can be induced to perform homologous recombination. Among these, established cell lines or primary cells, derived from either normal or diseased tissues (including cancer) can be included. Non-limiting examples of such human cells are: human cells immortalized by any methods (e.g. hTERT, HPV (Human Papilloma Virus), Large and small T antigen, SV40 (Simian Virus 40), E6 or E7 protein) and derived from different organ or tissues (e.g. breast, prostate, lung, bronchus, ovary, pancreas, liver, skin, kidney, uterus, stomach, esophagus, pharynx, larynx, bone, muscle, brain, cervix, blood, retina, colon-rectum, bladder, gallbladder, spleen) and at any level of differentiation (from stem to fully differentiated status).

In table 1 is provided a list of cell lines that can be used in the present invention.

TABLE 1

| Cell Line | Cell Bank | Catalogue number |
|---|---|---|
| NuLi, | ATCC | CRL-4011 |
| CuFi | ATCC | CRL-4013 |
| CHON-001 | ATCC | CRL-2846 |
| CHON-002 | ATCC | CRL-2847 |
| BJ-5ta | ATCC | CRL-4001 |
| hTERT-HME1 | ATCC | CRL-4010 |
| (ME16C) | | |
| hTERT RPE-1 | ATCC | CRL-4000 |
| hTERT-HPNE | ATCC | CRL-4023 |
| NeHepLxHT | ATCC | CRL-4020 |
| T HESCs | ATCC | CRL-4003 |
| RWPE-1 | ATCC | CRL-11609 |
| RWPE-2 | ATCC | CRL-11610 |
| WPE-stem | ATCC | CRL-2887 |
| WPE-int | ATCC | CRL-2888 |
| WPE1-NA22 | ATCC | CRL-2849 |
| WPE1-NB14 | ATCC | CRL-2850 |
| WPE1-NB11 | ATCC | CRL-2851 |
| WPE1-NB26 | ATCC | CRL-2852 |
| RWPE2-W99 | ATCC | CRL-2853 |
| WPMY-1 | ATCC | CRL-2854 |
| WPE1-NB26-64 | ATCC | CRL-11609 |
| WPE1-NB26-65 | ATCC | CRL-11610 |
| HBE4-E6/E7 | ATCC | CRL-2078 |
| [NBE4-E6/E7] | | |
| JVM-13 | ATCC | CRL-3003 |
| MeT-5A | ATCC | CRL-9444 |
| BBM | ATCC | CRL-9482 |
| BZR | ATCC | CRL-9483 |
| BEAS-2B | ATCC | CRL-9609 |
| MCF 10A | ATCC | CRL-10317 |
| MCF 10F | ATCC | CRL-10318 |
| MCF-10-2A | ATCC | CRL-10781 |
| B-3 | ATCC | CRL-11421 |
| HBE4-E6/E7-C1 | ATCC | CRL-2079 |
| HK-2 | ATCC | CRL-2190 |
| CHON-001 | ATCC | CRL-2846 |
| CHON-002 | ATCC | CRL-2847 |
| HS-5 | ATCC | CRL-11882 |
| PWR-1E | ATCC | CRL-11611 |
| THLE-3 | ATCC | CRL-11233 |
| HCE-2 [50.B1] | ATCC | CRL-11135 |
| 46BR.1N | ECACC-HPA | 92100623 |
| BRISTOL 8 | ECACC-HPA | 5011436 |
| AGLCL | ECACC-HPA | 89120566 |
| C211 | ECACC-HPA | 90112604 |
| GM1899A | ECACC-HPA | 98120701 |
| GS-109-V-63 | ECACC-HPA | 90110503 |
| GS-109-V-34 | ECACC-HPA | 90110504 |
| H9 | ECACC-HPA | 85050301 |
| HFFF2 | ECACC-HPA | 86031405 |
| HFL1 | ECACC-HPA | 89071902 |
| HG261 | ECACC-HPA | 90112603 |
| HH-8 | ECACC-HPA | 99090226 |
| HL | ECACC-HPA | 96121720 |
| Hs 68 | ECACC-HPA | 89051701 |
| Hs 888Lu | ECACC-HPA | 90112709 |
| Hs1.Tes | ECACC-HPA | 97123004 |
| IM 9 | ECACC-HPA | 86051302 |
| MRC-5 pd19 | ECACC-HPA | 05072101 |
| MRC-5 pd25 | ECACC-HPA | 05081101 |
| MRC-5 pd30 | ECACC-HPA | 84101801 |
| MRC-5 pd30 | ECACC-HPA | 05090501 |
| MRC-5 SV1 TG1 | ECACC-HPA | 85042501 |
| MRC-5 SV1 TG2 | ECACC-HPA | 85042502 |
| MRC-5 SV2 | ECACC-HPA | 84100401 |
| MRC-7 | ECACC-HPA | 85020203 |
| MRC-9 | ECACC-HPA | 85020202 |
| MT-2 | ECACC-HPA | 93121518 |
| PNT1A | ECACC-HPA | 95012614 |
| PNT1A (SERUM | ECACC-HPA | 07052901 |
| FREE) | | |
| PNT2 | ECACC-HPA | 95012613 |
| PNT2 (SERUM | ECACC-HPA | 07042701 |
| FREE) | | |
| SVCT | ECACC-HPA | 94122105 |
| SVCT-MI2 | ECACC-HPA | 98031105 |
| TK6 | ECACC-HPA | 95111735 |
| TK6TGR | ECACC-HPA | 87020507 |
| TOU (TOU I-2) | ECACC-HPA | 93093001 |
| WI 26 VA4 | ECACC-HPA | 89101301 |
| WI 38 | ECACC-HPA | 90020107 |
| WI 38VA13 | ECACC-HPA | 85062512 |
| Subline 2RA | | |
| WiDr | ECACC-HPA | 85111501 |
| WIL2 NS | ECACC-HPA | 90112121 |
| WIL2.NS.6TG | ECACC-HPA | 93031001 |
| WILCL | ECACC-HPA | 89120565 |
| OVCAR-5 | COSMIC | 875861 |
| OVCAR-4 | COSMIC | 688105 |
| OVCAR-3 | ATCC | HTB-161 |
| NCI-H522 | ATCC | CRL-5810 |
| NCI-H460 | ATCC | HTB-177 |
| NCI-H322M | COSMIC | 905967 |
| NCI-H23 | ATCC | CRL-5800 |
| NCI-H226 | ATCC | CRL-5826 |
| NCI/ADR-RES | COSMIC | 905987 |
| MOLT-4 | ATCC | CRL-1582 |
| MDA-N | | not available |
| MDA-MB-435 | COSMIC | 905988 |
| MDA-MB-231 | ATCC | HTB-26 |
| MCF7 | ATCC | HTB-22 |
| Malme-3M | ATCC | HTB-64 |
| M14 | COSMIC | 974261 |
| LOXIMV1 | COSMIC | 905974 |
| KM12 | COSMIC | 974247 |
| K-562 | | CCL-243 |
| IGROV1 | COSMIC | 905968 |
| HT-29 | ATCC | HTB-38 |
| Hs 578T | ATCC | HTB-126 |
| HOP-92 | COSMIC | 905973 |
| HOP-62 | COSMIC | 905972 |
| HL-60 | ATCC | CCL-240 |
| HCT-15 | ATCC | CCL-225 |
| HCT-116 | ATCC | CCL-247 |
| HCC-2998 | COSMIC | 905971 |
| EKVX | COSMIC | 905970 |
| DU-145 | ATCC | HTB-81 |
| COLO-205 | ATCC | CCL-222 |
| CCRF-CEM | ATCC | CCL-119 |
| CAKI-1 | ATCC | HTB-46 |
| BT-549 | ATCC | HTB-122 |
| ACHN | ATCC | CRL-1611 |
| A549 | ATCC | CCL-185 |
| A498 | ATCC | HTB-44 |
| 786-0 | ATCC | CRL-1932 |

Specifically, the present inventors focused on EGFR, KRAS, BRAF, PTEN, CTNNB1 and PIK3CA mutated alleles that are found in multiple cancer types and affect hundreds of thousands of patients currently suffering from this disease worldwide. In particular, the isogenic cell lines carried mutations frequently found in human tumors such as KRAS G13D, BRAF V600E, EGFR delE746-A750, CTNNB1 T41A, PTEN R130* and the PIK3CA mutations E545K and H1047R, that can be present alone or in combination between them. In addition to the above mentioned mutations all the cancer alleles listed in table 2a can be used to generate isogenic human cell lines carrying one or more mutated cancer alleles.

The derivative cell lines stringently recapitulate the molecular alterations present in human tumors, in that the mutated alleles are present in the heterozygous state and are regulated under the control of the targeted cells endogenous promoters. These mutant cells have then been used to study the biochemical, biological and transforming potential of common cancer alleles, to provide new insights into the molecular basis of cellular transformation and most of all to identify genotype-specific pharmacological profiles.

Several studies have shown that single cancer alleles—when ectopically expressed—can transform human cells.

In contrast, the present inventors found that the introduction of one or more cancer alleles in the genome of immortalized human cells of epithelial origin through the KI strategy was generally not sufficient to confer transforming properties. Thus, they postulated that the sequential addition of multiple mutations by direct modification of the corresponding genomic loci should prospectively allow the identification of the minimal number of genetic alterations required to transform human epithelial cells.

Understanding how the presence of common oncogenic alleles affects resistance and/or sensitivity to targeted drugs is key to define individualized cancer therapies. To address this issue the present inventors evaluated the response of the KI cells to a panel of over 90 compounds, including established (FDA approved) drugs and recently developed kinase inhibitors, using a proliferation screening assay. The profiling of drugs on the KI cells was highly informative on multiple levels. The ‘oncogene addiction’ phenotype displayed by EGFR mutant cells with EGFR kinase inhibitors such as erlotinib and gefitinib was unequivocal. On the contrary, and in accordance with recent clinical data, these drugs did not significantly affect growth of the isogenic clones in which the mutation of the EGFR was present together with a mutation leading to constitutive activation of the PIK3CA pathway downstream.

More detailed determining of the drug sensitivity data using a novel approach based on an algorithm designed to detect the hierarchical clustering of pharmacological profiles (pharmarrays) revealed other pathway interactions and sensitivity profiles of note.

Analysis of the hierarchical tree enlightened the proximity of the KRAS and BRAF phenotypes. Unequivocal biochemical, biological and genetic evidences had previously established that KRAS and BRAF act within the same signaling pathway. By using the pharmarray approach the present inventors demonstrated that combinatorial pharmacogenomic analysis of cells carrying activating alleles for these two genes identifies cancer mutations likely to act in the same or overlapping signaling pathways.

If systematically applied to cells carrying newly discovered cancer alleles, this approach can lead to pharmarray based charts of the pathways in which the individual mutations are implicated.

The signaling network centered on the lipid kinase PIK3CA is deregulated in many tumor types and is currently the focus of multiple therapeutic efforts in light of its ‘druggability’. The present disclosure allows a more detailed analysis of the drugs showing an ‘oncogene addiction’ phenotype towards the PIK3CA mutated cells. The present experiments revealed that everolimus had a striking selectivity for non-tumorigenic cells carrying PIK3CA mutations.

Everolimus is currently the focus of extensive oncology clinical trials; the relationship between PIK3CA mutations and sensitivity to everolimus was investigated in human cancer cells. Using a panel of cell lines derived from various tumor lineages and carrying genetic alterations in members of the PIK3CA pathway, two groups were identified based on their response to everolimus. Intriguingly, everolimus-resistant cells, in addition to PIK3CA mutations, also carried KRAS oncogenic alleles. In these cells the genetic removal of the KRAS mutated (but not of the WT) allele restored sensitivity to everolimus. Furthermore, in these cells combinatorial pharmacological targeting of the KRAS and PIK3CA pathways had a synergistic pattern confirming the genetic-based observation. These data indicate that the oncogenic status of KRAS plays a central role in conferring resistance to the antiproliferative effects of everolimus in tumor cells harbouring genetic alterations in the PIK3CA gene. The present inventors also verified that the findings obtained in knock-in (KI) cell models were reproducible in cancer cells in which the corresponding mutations naturally occur.

These findings have enormous implications for genetically driven selection of cancer patients currently undergoing clinical trials with everolimus and for the rationale interpretation of their treatment outcome.

Importantly, the pharmarray analysis detected pharmacological relationships for the KI cells equivalent to those for cancer cells in which the corresponding mutations naturally occur.

Therefore, the present results indicate that KI cell models can be successfully used to evaluate how oncogenic alleles affect resistance/sensitivity to anticancer therapies.

A number of general considerations can be drawn from these results.

KI of cancer mutations generates cellular models in which the mutated genes are expressed under their endogenous promoters, closely recapitulating the lesions observed in human tumors. While the mutant cells display allele-specific biochemical and biological properties, they are not transformed. The present process allows, thus, to pave the way to the identification of the number and sequential order of genetic lesions required to transform human epithelial cells. The present process allows, also, to establish which of the hundreds of alleles recently identified by the cancer genome projects act as ‘drivers’ or ‘passengers’ with respect to tumorigenesis (4, 5).

Mutant cells show striking ‘oncogene addiction’ phenotypes, either enhanced sensitivity or resistance, when treated with targeted inhibitors resembling the response and resistance mechanisms occurring in human tumors. Profiling of bioactive drugs on KI cells can be rapidly performed to identify drug-genotype correlations thus allowing the rational design of clinical trials based on the genetic milieu of individual tumors.

In order to distinguish and track KI cells both during in vitro and in vivo (on laboratory animal models) assays, it is very useful to label them with molecules selected among fluorescent, radioactive, luminescent, phosphorescent markers.

Retroviral or lentiviral vectors expressing one of the above mentioned molecules can be generated and used to infect the isogenic cell line of interest. Clones expressing the marker molecule at the desired intensity can be isolated and used alone or in combination with differently marked clones for the assays.

Non-limiting examples for possible applications are:
in vitro drug resistance/sensitivity assay: wt and KI cells marked with different tracing agents can be mixed in the same plate and then undergo drug treatment. Resistant cells surviving drug exposure can be monitored through microscope analysis.
xenograft model (where a xenograft consists of living cells, tissues or organs, that are xenotransplanted from one species to another such as from human to mouse) of tumorigenesis: cell lines expressing a molecular marker can be injected subcutaneously in the flank of a laboratory animal model, e.g. a mouse, thus giving rise to tumors. The growth and the dissemination of these cells can then be in vivo monitored through the use of special instrumentation such us microscopes or camera for detection of fluorescent, radioactive, luminescent, phosphorescent markers known to the person skilled in the field.

With a similar strategy, it is possible to measure the in vivo effect of drug treatment, where the fate of these KI treated cells can be monitored in real-time thanks to the employment of these biomarkers. It is important to note that while a tumor cell line is able to give rise to xenograft tumor in mice, a non-transformed cell line can become tumorigenic by the expression of multiple oncogenic alleles (such as for example KRAS, BRAF, EGFR, PIK3CA, PTEN, CTNNB1, c-KIT, c-MET, EPHA3, Erbb2, AKT1, FGFR2, MSH6, ABL1, STAT1, STAT4, RET, AKT3, TEK, VAV3, ALK, LYN, NOTCH, IDH1, ROR1, FLT3, ALK, SRC, BCL9, RPS6KA2, PDPK1, NTRK3, NTRK2, AKT3, KDR, MKK4, FBWX7, MEK1, OBSCN, TECTA, MLL3, NRAS, HRAS, TP53, APC, Rb1, CDKN2A (p16), BRCA1, BRCA2, PTCH1, VHL, SMAD4, PER1, MEN1, NF1, NF2, ATM, PTPRD, see table 2a) and/or the concomitant inactivation of tumor suppressor genes including but not limited to p53, p21, Rb1, PTEN, APC, BUB1, BRCA1, BRCA2, PTCH, VHL, SMAD4, PER1, TSC2, CDKN2A, DCC, MEN-1, NF1, ATM, PTPRD, LRP1B and NF2 (see table 2b).

As a different application of isogenic cells, a reporter gene, selected among fluorescent, radioactive, luminescent, phosphorescent markers, can be introduced in-frame to monitor the level of expression of the target allele.

The reporter gene is placed through homologous recombination at the 3′ end of the allele of interest, so that its expression is driven by the same endogenous promoter regulating the expression of the target allele. Moreover, using two different reporters, it is possible to track at the same time both alleles (wt and KI), thus evaluating the specific contribution of both of them to any observed phenotype.

### Materials and Methods

### Cells and Cell Culture Reagents

The following cell lines were purchased from American Type Culture Collection (ATCC, Manassas, Va.): hTERT-HME1 (ATCC® CRL-4010™), MCF10A (ATCC® CRL-10317™), hTERT RPE-1 (ATCC® CRL-4000™) and SW48 (ATCC® CCL-231). hTERT-HME1 and MCF10A were cultured in growth medium containing DMEM/F-12 (Invitrogen Carlsbad, Calif.) supplemented with 20 ng/mL epidermal growth factor (EGF), 10 μg/mL insulin and 100 μg/mL hydrocortisone. DLD-1 and SW48 cells were cultured in DMEM (Invitrogen, Carlsbad, Calif.), while hTERT RPE-1 cells were grown in RPMI-1640 medium (Invitrogen, Carlsbad, Calif.). All cell culture media were supplemented with 10% fetal bovine serum (Sigma-Aldrich, St. Louis, Mo.), 50 units/mL penicillin and 50 mg/mL streptomycin. Geneticin (G418) was purchased from Gibco (Carlsbad, Calif.). Isogenic HCT 116 and DLD-1 PIK3CA WT and mutant cells were generously provided by the Vogelstein/Velculescu laboratories (6). All other cancer cell lines (U-87 MG-ATCC® HTB-14, Ca Ski-ATCC® CRL-1550, ME-180-ATCC® HTB-33, MCF7-ATCC® HTB-22, BT-474-ATCC® HBT-20, PC-3-ATCC® CRL-1435, PANC-1-ATCC® CRL-1469, HT-29-ATCC® HTB-38, NIH:OVCAR-3-ATCC® HTB-161, SK-OV-3-ATCC® HTB-77, HCT 116-ATCC® CCL-247 and DLD-1-ATCC® CCL-221) were obtained from ATCC and cultured according to their recommendations.

### Cancer Alleles

The nucleotide sequences of the wild-type alleles used in the present disclosure (i.e. BRAF, EGFR, KRAS, PIK3CA, PTEN and CTNNB1) are public available in GenBank and the corresponding reference numbers are provided in table 2a, as well as the nucleotide sequences of the mutated BRAF, EGFR, KRAS, PIK3CA, PTEN and CTNNB1 allele exons (SEQ ID NO.: 1 to 7) used in the present disclosure.

The other cancer alleles listed in Table 2a are encompassed by the present disclosure and can be used, according to the teaching provided herein, for generating different isogenic human cell lines carrying—by means of the knock-in strategy—one or more of the mutated cancer alleles different from those cited above.

TABLE 2a

| | GenBank | |
|---|---|---|
| Allele | wild-type | Allele mutation |
| PIK3CA | NM_006218 | Exon 9 mutated G1633A (E545K) |
| | | SEQ ID No.: 1 |
| | | Exon 20 mutated A3140G (H1047R) |
| | | SEQ ID No.: 2 |
| BRAF | NM_004333 | Exon 15 mutated T1799A (V600E) |
| | | SEQ ID No.: 3 |
| KRAS | NM_004985 | Exon 2 mutated G38A (G13D) |
| | | SEQ ID No.: 4 |
| EGFR | NM_005228 | Exon 19 mutated del 2235- |
| | | GGAATTAAGAGAAGC-2249 |
| | | (delE746-A750) |
| | | SEQ ID No.: 5 |
| CTNNB1 | NM_001098210 | Exon 3 mutated C121G (T41A) |
| | | SEQ ID No.: 6 |
| PTEN | NM_000314 | Exon 5 mutated C388T (R130*) |
| | | SEQ ID No.: 7 |
| | | (The asterisk indicate a STOP codon) |
| KRAS | NM_004985 | c.32C > T; p.A11V |
| | | c.35G > C; p.G12A |
| | | c.34G > T; p.G12C |
| | | c.35G > A; p.G12D |
| | | c.34_35GG > TT; p.G12F |
| | | c.36T > C; p.G12G |
| | | c.34_35GG > CC; p.G12L |
| | | c.34G > C; p.G12R |
| | | c.34G > A; p.G12S |
| | | c.38G > C; p.G13A |
| | | c.37G > T; p.G13C |
| | | c.38_39GC > AT; p.G13D |
| | | c.38G > A; p.G13D |
| | | c.39C > T; p.G13G |
| | | c.37G > C; p.G13R |
| | | c.37G > A; p.G13S |
| | | c.38G > T; p.G13V |
| | | c.40G > A; p.V14I |
| | | c.57G > C; p.L19F |
| | | c.64C > A; p.Q22K |
| | | c.175G > A; p.A59T |
| | | c.181C > G; p.Q61E |
| | | c.183A > C; p.Q61H |
| | | c.183A > T; p.Q61H |
| | | c.181C > A; p.Q61K |
| | | c.182A > T; p.Q61L |
| | | c.182A > C; p.Q61P |
| | | c.182A > G; p.Q61R |
| | | c.436G > A; p.A146T |
| | | c.36_37insGGT; p.G12_G13insG |
| NRAS | NM_002524 | c.29G > A; p.G10E |
| | | c.31G > A; p.A11T |
| | | c.35G > C; p.G12A |
| | | c.34G > T; p.G12C |
| | | c.35G > A; p.G12D |
| | | c.34_35GG > AA; p.G12N |
| | | c.34_35GG > CC; p.G12P |
| | | c.34G > C; p.G12R |
| | | c.34G > A; p.G12S |
| | | c.35G > T; p.G12V |
| | | c.34_35GG > TA; p.G12Y |
| | | c.38G > C; p.G13A |
| | | c.37G > T; p.G13C |
| | | c.38G > A; p.G13D |
| | | c.37G > C; p.G13R |
| | | c.37G > A; p.G13S |
| | | c.38G > T; p.G13V |
| | | c.181C > G; p.Q61E |
| | | c.183A > T; p.Q61H |
| | | c.183A > C; p.Q61H |
| | | c.181C > A; p.Q61K |
| | | c.180_181AC > TA; p.Q61K |
| | | c.182A > T; p.Q61L |
| | | c.181_182CA > TT; p.Q61L |
| | | c.182A > C; p.Q61P |
| | | c.183A > G; p.Q61Q |
| | | c.181_182CA > AG; p.Q61R |
| | | c.182A > G; p.Q61R |
| | | c.190T > A; p.Y64N |
| | | c.193A > T; p.S65C |
| HRAS | NM_005343 | c.31G > T; p.A11S |
| | | c.34G > T; p.G12C |
| | | c.35G > C; p.G12A |
| | | c.35G > A; p.G12D |
| | | c.34G > C; p.G12R |
| | | c.34G > A; p.G12S |
| | | c.35G > T; p.G12V |
| | | c.37G > T; p.G13C |
| | | c.38G > A; p.G13D |
| | | c.37G > C; p.G13R |
| | | c.37G > A; p.G13S |
| | | c.38G > T; p.G13V |
| | | c.49A > G; p.S17G |
| | | c.52G > A; p.A18T |
| | | c.59C > T; p.T20I |
| | | c.64C > T; p.Q22* |
| | | c.175G > A; p.A59T |
| | | c.181C > G; p.Q61E |
| | | c.183G > T; p.Q61H |
| | | c.183G > C; p.Q61H |
| | | c.181C > A; p.Q61K |
| | | c.182A > T; p.Q61L |
| | | c.182A > C; p.Q61P |
| | | c.182A > G; p.Q61R |
| | | c.182_183AG > GT; p.Q61R |
| BRAF | NM_004333 | c.1391G > A; p.G464E |
| | | c.1390G > C; p.G464R |
| | | c.1391G > T; p.G464V |
| | | c.1397G > T; p.G466V |
| | | c.1397G > A; p.G466E |
| | | c.1397G > C; p.G466A |
| | | c.1396G > C; p.G466R |
| | | c.1406G > C; p.G469A |
| | | c.1406G > A; p.G469E |
| | | c.1405G > A; p.G469R |
| | | c.1405_1406GG > TC; p.G469S |
| | | c.1406G > T; p.G469V |
| | | c.1781A > G; p.D594G |
| | | c.1786G > C; p.G596R |
| | | c.1790T > A; p.L597Q |
| | | c.1790T > G; p.L597R |
| | | c.1789_1790CT > TC; p.L597S |
| | | c.1789C > G; p.L597V |
| | | c.1799T > C; p.V600A |
| | | c.1799_1800TG > AT; p.V600D |
| | | c.1798_1799GT > AA; p.V600K |
| | | c.1798G > A; p.V600M |
| | | c.1798_1799GT > AG; p.V600R |
| | | c.1801A > G; p.K601E |
| EGFR | NM_005228 | c.323G > A; p.R108K |
| | | c.866C > T; p.A289V |
| | | c.2030G > A; p.R677H |
| | | c.2125G > A; p.E709K |
| | | c.2126A > C; p.E709A |
| | | c.2126A > G; p.E709G |
| | | c.2155G > A; p.G719S |
| | | c.2155G > T; p.G719C |
| | | c.2156G > C; p.G719A |
| | | c.2156G > A; p.G719D |
| | | c.2303G > T; p.S768I |
| | | c.2326C > T; p.R776C |
| | | c.2369C > T; p.T790M |
| | | c.2497T > G; p.L833V |
| | | c.2573T > G; p.L858R |
| | | c.2582T > A; p.L861Q |
| | | c.2582T > G; p.L861R |
| | | c.2235_2249del15; p.E746_A750del |
| | | c.2236_2250del15; p.E746_A750del |
| | | c.2237_2251del15; p.E746_T751 > A |
| | | c.2239_2256del18; p.L747_S752del |
| | | c.2240_2257del18; p.L747_P753 > S |
| | | c.2240_2254del15; p.L747_T751del |
| | | c.2237_2255 > T; p.E746_S752 > V |
| | | c.2239_2248TTAAGAGAAG > C; |
| | | p.L747_A750 > P |
| | | c.2239_2251 > C; p.L747_T751 > P |
| PIK3CA | NM_006218.1 | c.(1624_1633)G > A; |
| | | p.(542_545)E > K |
| | | c.113G > A; p.R38H |
| | | c.263G > A; p.R88Q |
| | | c.277C > T; p.R93W |
| | | c.317G > T; p.G106V |
| | | c.323G > A; p.R108H |
| | | c.331A > G; p.K111E |
| | | c.333G > C; p.K111N |
| | | c.353G > A; p.G118D |
| | | c.1035T > A; p.N345K |
| | | c.1132T > C; p.C378R |
| | | c.1357G > A; p.E453K |
| | | c.1616C > G; p.P539R |
| | | c.1625A > G; p.E542G |
| | | c.1624G > A; p.E542K |
| | | c.1624G > C; p.E542Q |
| | | c.1625A > T; p.E542V |
| | | c.1633G > C; p.E545Q |
| | | c.1634A > C; p.E545A |
| | | c.1634A > G; p.E545G |
| | | c.1634A > T; p.E545V |
| | | c.1635G > T; p.E545D |
| | | c.1636C > G; p.Q546E |
| | | c.1636C > A; p.Q546K |
| | | c.1637A > T; p.Q546L |
| | | c.1637A > C; p.Q546P |
| | | c.1638G > T; p.Q546H |
| | | c.1637A > G; p.Q546R |
| | | p.Q546R; p.D549N |
| | | c.2102A > C; H701P |
| | | c.3019G > C; p.G1007R |
| | | c.3061T > C; p.Y1021H |
| | | c.3062A > G; p.Y1021C |
| | | c.3061T > A; p.Y1021N |
| | | c.3073A > G; p.T1025A |
| | | c.3074C > A; p.T1025N |
| | | c.3073A > T; p.T1025S |
| | | c.3075C > T; p.T1025T |
| | | c.3129G > T; p.M1043I |
| | | c.3127A > G; p.M1043V |
| | | c.3132T > A; p.N1044K |
| | | c.3133G > A; p.D1045N |
| | | c.3136G > A; p.A1046T |
| | | c.3140A > T; p.H1047L |
| | | c.3139C > T; p.H1047Y |
| | | c.3145G > C; p.G1049R |
| | | c.3145G > A; p.G1049S |
| | | c.3155C > A; p.T1052K |
| | | c.3194A > T; p.H1065L |
| | | c.3204_3205insA; p.N1068fs*4 |
| CTNNB1 | NM_001904 | c.86C > T; p.S29F |
| | | c.95A > C; p.D32A |
| | | c.95A > G; p.D32G |
| | | c.94G > C; p.D32H |
| | | c.94G > A; p.D32N |
| | | c.95A > T; p.D32V |
| | | c.94G > T; p.D32Y |
| | | c.97T > G; p.S33A |
| | | c.98C > G; p.S33C |
| | | c.98C > T; p.S33F |
| | | c.97_98TC > CT; p.S33L |
| | | c.97T > C; p.S33P |
| | | c.98C > A; p.S33Y |
| | | c.101G > A; p.G34E |
| | | c.100G > C; p.G34R |
| | | c.100G > A; p.G34R |
| | | c.101G > T; p.G34V |
| | | c.104T > G; p.I35S |
| | | c.107A > C; p.H36P |
| | | c.106C > T; p.H36Y |
| | | c.109T > G; p.S37A |
| | | c.110C > G; p.S37C |
| | | c.110C > T; p.S37F |
| | | c.109T > C; p.S37P |
| | | c.110C > A; p.S37Y |
| | | c.112_114GGT > CCC; p.G38P |
| | | c.119C > T; p.T40I |
| | | c.122C > T; p.T41I |
| | | c.122C > G; p.T41S |
| | | c.130C > G; p.P44A |
| | | c.130C > T; p.P44S |
| | | c.133T > G; p.S45A |
| | | c.134C > G; p.S45C |
| | | c.134C > T; p.S45F |
| | | c.133T > C; p.S45P |
| | | c.134C > A; p.S45Y |
| | | c.140G > A; p.S47N |
| | | c.143G > A; p.G48D |
| | | c.143G > T; p.G48V |
| | | c.146A > G; p.K49R |
| | | c.157G > A; p.E53K |
| | | c.172G > A; p.D58N |
| | | c.74_97del24; p.W25_D32del |
| c-KIT | NM_001093772 | c.1727T > C; p.L576P |
| | | c.154G > A; p.D52N |
| | | c.1676T > A; p.V559D |
| | | c.1676T > C; p.V559A |
| | | c.1676T > G; p.V559G |
| | | c.1679T > A; p.V560D |
| | | c.1679T > G; p.V560G |
| | | c.1681G > A; p.E561K |
| | | c.1924A > G; p.K642E |
| | | c.1961T > C; p.V654A |
| | | c.2446G > C; p.D816H |
| | | c.2446G > T; p.D816Y |
| | | c.2447A > T; p.D816V |
| | | c.2467T > G; p.Y823D |
| | | c.2474T > C; p.V825A |
| | | c.1509_1510insGCCTAT; |
| | | p.Y503_F504insAY |
| | | c.1669_1674delTGGAAG; |
| | | p.W557_K558del |
| | | c.1675_1677delGTT; p.V559del |
| | | c.1735_1737delGAT; p.D579del |
| c-MET | NM_000245 | c.504G > T; p.E168D |
| | | c.687G > T; p.L229F |
| | | c.849C > T; p.S283S |
| | | c.1124A > G; p.N375S |
| | | c.1128G > A; p.K376K |
| | | c.2962C > T; p.R988C |
| | | c.468G > A p.S156S |
| | | c.3757T > G p.Y1253D |
| | | c.3029C > T; p.T1010I |
| | | c.3803T > C; p.M1268T |
| | | c.3743A > G; p.Y1248C |
| EPHA3 | NM_005233 | c.686C > A; p.S229Y |
| | | c.1346C > T; p.S449F |
| | | c.2297G > A; p.G766E |
| | | c.1552_1553GG > TT; p.G518L |
| | | c.110C > A p.T37K |
| | | c.254A > G p.N85S |
| | | c.1861A > C p.I621L |
| | | c.2416G > A p.D806N |
| | | c.907 G > A p.G228R |
| | | c.1725G > T p.K500N |
| | | c.3136 G > C p.A971AP |
| Erbb2 | NM_004448 | c.2264T > C; p.L755S |
| | | c.2305G > C; p.D769H |
| | | c.2326G > A; p.G776S |
| | | c.2172G > T p.K724N |
| | | c.2198C > T p.T733I |
| | | c.2263_2264TT > CC; p.L755P |
| | | c.2327G > T; p.G776V |
| | | c.2329G > T; p.V777L |
| | | c.2524G > A; p.V842I |
| | | c.2632C > T; p.H878Y |
| | | c.2322_2323ins12; |
| | | p.M774_A775insAYVM |
| | | c.2324_2325ins12; |
| | | p.A775_G776insYVMA |
| AKT1 | NM_005163 | c.49 G > A p.E17K |
| FGFR2 | CCDS7620.1 | c.607C > T p.R203C |
| PDGFRB | NM_002609 | c.1765T > C p.Y589H |
| | | c.2645C > T p.T882I |
| | | c.3270G > A p.P1090P |
| MSH6 | NM_000179 | c.3246G > T p.P1082P |
| ABL1 | NM_007313. | c.1052T > C p.M351T |
| STAT1 | CCDS2309.1 | c.1471C > G p.P491A |
| STAT4 | CCDS2310.1 | c.334G > C p.E112Q |
| RET | NM_020975 | c.2753T > C p.M918T |
| | | c.434T > G p.V145G |
| | | c.1078C > T p.R360W |
| | | c.1778G > A p.G593E |
| AKT3 | NM_005465 | c.511G > A p.G171R |
| TEK | CCDS6519.1 | c.351G > C p.K117N |
| VAV3 | CCDS785.1 | c.1153C > T p.Q385X |
| LYN | NM_002350 | c.1153G > T p.D385Y |
| NOTCH | NM_017617 | c.3647G > A p.G1216D |
| | | c.2912C > T p.T971I |
| | | c.1858G > T p.D620Y |
| IDH1 | NM_005896.2 | c.394C > T p.R132C |
| | | c.394C > A p.R132S |
| | | c.395G > A p.R132H |
| ROR1 | NM_005012 | c.448T > C p.F150L |
| | | c.1700G > T p.R567I |
| | | c.2181G > A p.E727E |
| | | c.2667A > G p.S889S |
| FLT3 | U02687 | c.2503G > C p.D835H |
| ALK | NM_004304 | c.3502G > C p.A1168P |
| | | c.2269G > A p.V757M |
| | | c.1202G > A p.R401Q |
| SRC | NM_005417 | c.1591C > T p.Q531* |
| BCL9 | NM_004326 Mx38 | c.3664G > T p.E1222X |
| RPS6KA2 | NM_021135 | c.1280C > G p.S427* |
| | | c.2195G > A p.R732Q |
| PDPK1 | NM_002613 | c.282C > T p.S94S |
| NTRK3 | NM_002530 | c.2029C > T p.H677Y |
| | | c.1464C > T p.I488I |
| | | c.919G > C p.V307L |
| NTRK2 | NM_006180 | c.412C > T p.L138F |
| | | c.2263C > T p.L755L |
| | | c.2442G > A p.K814K p.K814K |
| KDR | NM_002253 | c.743C > G p.A248G |
| MKK4 | NM_003010 | c.929G > A p.W310* |
| | | c.425A > T p.Q142L |
| FBWX7 | NM_033632.2 | c.1745C > T p.S582L |
| | | c.1514G > T p.R505L |
| | | c.1394G > A p.R465H |
| MEK1 | NM_002755 | c.171 G > T K57N |
| | | c.199 G > A D67N |
| OBSCN | CCDS1570.1 | c.15211G > A p.A5071T |
| | | c.11453G > A p.G3818E |
| | | c.13791G > A p.E4574K |
| TECTA | NM_005422.2 | c: 2404 C > T p.P802S |
| MLL3 | NM_170606.2 | C: 5767 C > G p.P1863A |
| | | c.11020-11022delGAT p.3614Ddel |
| PTEN | NM_000314.4 | c.388C > T; p.R130* |
| | | c.388C > G; p.R130G |
| | | c.389G > A; p.R130Q |
| | | c.513G > C; p.Q171H |
| | | c.518G > A; p.R173H |
| | | c.697C > T; p.R233* |
| | | c.1003C > T; p.R335* |
| TP53 | NM_000546 | c.524G > A; p.R175H |
| | | c.659A > G; p.Y220C |
| | | c.734G > T; p.G245V |
| | | c.743G > T; p.R248L |
| | | c.743G > A; p.R248Q |
| | | c.742C > T; p.R248W |
| | | c.818G > A; p.R273H |
| | | c.817C > T; p.R273C |
| | | c.818G > T; p.R273L |
| APC | NM_000038 | c.3340C > T; p.R1114* |
| | | c.4012C > T; p.Q1338* |
| | | c.4135G > T; p.E1379* |
| | | c.4348C > T; p.R1450* |
| Rb1 | NM_000321 | c.160G > T; p.E54* |
| | | c.596T > A; p.L199* |
| | | c.958C > T; p.R320* |
| | | c.1072C > T; p.R358* |
| | | c.1363C > T; p.R455* |
| | | c.1654C > T; p.R552* |
| | | c.1666C > T; p.R556* |
| | | c.1735C > T; p.R579* |
| | | c.2117G > T; p.C706F |
| | | c.2242G > T; p.E748* |
| CDKN2A | NM_000077 | c.143C > T; p.P48L |
| (p16) | | c.170C > T; p.A57V |
| | | c.172C > T; p.R58* |
| | | c.181G > T; p.E61* |
| | | c.205G > T; p.E69* |
| | | c.238C > T; p.R80* |
| | | c.239G > A; p.R80Q |
| | | c.247C > T; p.H83Y |
| | | c.250G > T; p.D84Y |
| | | c.322G > T; p.D108Y |
| | | c.330G > A; p.W110* |
| | | c.341C > T; p.P114L |
| BRCA1 | NM_007294 | c.90G > T; p.L30F |
| | | c.340T > A; p.S114T |
| | | c.1116G > A; p.W372* |
| | | c.2269_2269delG; p.V757fs*8 |
| | | c.3026C > A; p.S1009* |
| | | c.5173G > T; p.E1725* |
| BRCA2 | NM_000059 | c.4550_4559del10; p.K1517fs*23 |
| | | c.5351delA; p.N1784fs*7 |
| | | c.1063G > C; p.V355L |
| | | c.1889C > T; p.T630I |
| | | c.4014C > T; p.G1338G |
| | | c.4777G > T; p.E1593* |
| | | c.5046T > C; p.S1682S |
| | | c.5962G > A; p.V1988I |
| | | c.7243C > A; p.H2415N |
| | | c.8360G > A; p.R2787H |
| | | c.8524C > T; p.R2842C |
| | | c.9285C > A; p.D3095E |
| | | c.9309A > G; p.I3103M |
| | | c.9382C > T; p.R3128* |
| | | c.10070C > G; p.T3357R |
| PTCH1 | NM_000264 | c.709G > A; p.E237K |
| | | c.1093C > T; p.Q365* |
| | | c.1247C > G; p.T416S |
| | | c.1249C > T; p.Q417* |
| | | c.1682T > G; p.M561R |
| | | c.2307_2308CC > TT; p.R770* |
| | | c.3054G > A; p.W1018* |
| | | c.3944T > C; p.L1315P |
| VHL | NM_000551 | c.559_560delGA; p.D187fs*27 |
| | | c.554delA; p.Y185fs*17 |
| | | c.524delA; p.Y175fs*27 |
| | | c.523delT; p.Y175fs*27 |
| | | c.514delC; p.P172fs*30 |
| | | c.501_501delG; p.S168fs*2 |
| | | c.469delA; p.T157fs*2 |
| | | c.444delT; p.F148fs*11 |
| | | c.439_440insT; p.A149fs*25 |
| | | c.548C > A; p.S183* |
| | | c.539T > A; p.I180N |
| | | c.194C > T; p.S65L |
| | | c.241C > T; p.P81S |
| | | c.240T > A; p.S80R |
| | | c.254T > C; p.L85P |
| | | c.203C > A; p.S68* |
| | | c.266T > A; p.L89H |
| | | c.340G > T; p.G114C |
| | | c.481C > T; p.R161* |
| | | c.473T > A; p.L158Q |
| | | c.472C > G; p.L158V |
| | | c.478G > A; p.E160K |
| SMAD4 | NM_005359 | c.733C > T; p.Q245* |
| | | c.1028C > G; p.S343* |
| | | c.1051G > C; p.D351H |
| | | c.989A > C; p.E330A |
| | | c.1081C > T; p.R361C |
| | | c.1082G > A; p.R361H |
| | | c.1156G > C; p.G386R |
| | | c.1333C > T; p.R445* |
| | | c.1394_1395insT; p.A466fs*28 |
| | | c.1546_1553delCAGAGCAT; |
| | | p.S517fs*7 |
| PER1 | ENST00000317276 | c.1411_1412insGT; F471fs*46 |
| | | c.652G > A; p.D218N |
| MEN1 | ENST00000312049 | c.266T > G; p.L89R |
| | | c.292C > T; p.R98* |
| | | c.378G > A; p.W126* |
| | | c.1413G > A; p.W471* |
| | | p.W471*; p.G208fs*16 |
| | | c.1033_1033delG; p.A345fs*23 |
| NF1 | ENST00000358273 | c.910C > T; p.R304* |
| | | c.1381C > T; p.R461* |
| | | c.4330A > G; p.K1444E |
| | | c.4330A > C; p.K1444Q |
| | | c.4600C > T; p.R1534* |
| | | c.4082_4083insT; p.R1362fs*18 |
| NF2 | NM_000268.2 | c.169C > T; p.R57* |
| | | c.432C > A; p.Y144* |
| | | c.459C > G; p.Y153* |
| | | c.586C > T; p.R196* |
| | | c.634C > T; p.Q212* |
| | | c.655G > A; p.V219M |
| | | c.784C > T; p.R262* |
| | | c.810G > T; p.E270D |
| | | c.1009C > T; p.Q337* |
| | | c.1021C > T; p.R341* |
| | | c.1198C > T; p.Q400* |
| | | c.1228C > T; p.Q410* |
| | | c.1396C > T; p.R466* |
| | | c.364_447del84; p.V122_K149del |
| ATM | NM_000051 | c.1009C > A; p.R337S |
| | | c.1810C > T; p.P604S |
| | | c.2572T > C; p.F858L |
| | | c.7328G > A; p.R2443Q |
| | | c.7996A > G; p.T2666A |
| | | c.8084G > C; p.G2695A |
| | | c.8174A > T; p.D2725V |
| | | c.8600G > A; p.G2867E |
| | | c.9022C > T; p.R3008C |
| | | c.9023G > A; p.R3008H |
| | | c.9139C > T; p.R3047* |
| PTPRD | NM_002839.1 | c.460G > T; p.D154Y |

Table 2b lists tumor suppressor genes which can be used to generate, according to the present disclosure, isogenic human cell lines carrying—together with at least one mutated cancer allele listed in table 2a—at least one knock-out or inactivated tumor suppressor gene e.g. for the production of xenografts.

TABLE 2b

| Tumor suppressor gene | GenBank |
|---|---|
| PTEN | NM_000314.4 |
| TP53 | NM_000546 |
| APC | NM_000038 |
| p21 | NM_000389 |
| Rb1 | NM_000321 |
| BUB1 | NM_004336 |
| BRCA1 | NM_007294 |
| BRCA2 | NM_000059 |
| PTCH1 | NM_000264 |
| VHL | NM_000551 |
| SMAD4 | NM_005359 |
| PER1 | ENST00000317276 |
| TSC2 | NM_000548 |
| CDKN2A | NM_000077 |
| MEN-1 | ENST00000312049 |
| NF1 | ENST00000358273 |
| ATM | NM_000051 |
| PTPRD | NM_002839.1 |
| NF2 | NM_000268.2 |

### Drug Assays

Parental and KI cells were seeded in 100 μL complete growth medium at appropriate density (1×10⁴, 4×10⁴, 5×10⁴, for hTERT RPE-1, hTERT-HME1 and MCF10A cells, respectively) in 96-well plastic culture plates. After serial dilutions, 100 μl of drugs in serum free medium were added to cells with a multichannel pipette. Vehicle and medium-only containing wells were added as controls. Plates were incubated at 37° C. in 5% CO₂ for 96 h, after which cell viability was assessed by ATP content using the CellTiter-Glo® Luminescent Assay (Promega Madison, Wis.). To account for clonal variability, multiple independent clones carrying each of the mutations were generated and analyzed. For refined analysis, all cells were stained with Hoechst 33342 1 μg/ml (Molecular Probes, Invitrogen, Milan, Italy) and the nuclei of dead cells were counterstained with propidium iodide 2 μg/ml (Molecular Probes, Invitrogen, Milan, Italy) for 30 minutes at 37° C. Cells were then washed in phenol-red-free RPMI 1640 and photographed with a BD-Pathway HT Bioimager.

### Flow Cytometric Analysis

For time-course experiments, on the initial day hTERT-HME1 cells were labelled with 3 μM CFSE (5-(and -6)-carboxyfluorescein diacetate, succinimidyl ester, Invitrogen C1157, Milan, Italy) in PBS in the dark for 30 minutes. After washing and recording baseline fluorescence, cells were plated in media containing 1% FBS and 2 ng/mL EGF, and treatment with everolimus was initiated, replenishing the drug on a daily basis. For cell cycle analysis, trypsinized cells were washed with PBS and cell nuclei DNA were stained with propidium iodide (PI) for at least 120 minutes using a commercial kit (DNA con 3, Consul T.S., Orbassano, Italy).

All fluorescence levels were detected by flow cytometry on a FACSCalibur (Becton Dickinson, Milan, Italy) and analyzed using CellQuest software. The number of events collected for each sample varied between 15,000 and 50,000. After doublets exclusion, an extended analysis of the DNA content and calculations of the percentage of cells in each phase of the cell cycle were performed on ModFit Lt software (Verity Software House, Topsham, Me.).

### Protein Analysis

SDS PAGE was performed using Invitrogen Precasted gels (Invitrogen Carlsbad, Calif.); western blotting transfer onto Hybond-C Extra membranes (Amersham, Amersham Biosciences, Uppsala, Sweden) was done following standard methods. The primary antibodies used for immunoblotting were: Anti-AKT (Cell Signaling, Technology, Danvers, Mass.); Anti-phospho-AKT S473 (Cell Signaling, Technology, Danvers, Mass.); Anti-Actin and Anti-Vinculin (Sigma-Aldrich, St. Louis, Mo.); Phospho-p44/42 Map kinase (Thr202/Tyr204) (Cell Signaling, Technology, Danvers, Mass.); Anti-phospho-EGFR Receptor (Tyr 1068) and Anti EGFR Receptor (Cell Signaling Technology, Danvers, Mass.).

### ELISA Assay (PIP3 Production)

WT and PIK3CA KI cells were starved for 72 h and a PI3K-ELISA assay (Echelon Biosciences Incorporated, Salt Lake City, Utah) was used to detect the levels of PI3-kinase activity, following manufacturer instructions.

### Ras Activation Assay

GST-RAF-RAS binding domain fusion proteins conjugated with agarose beads were purchased from Upstate Biotechnology (Raf-1-GST Ras Binding Domain, Catalog #14-278, Upstate Biotechnology, Lake Placid, N.Y.). HCT 116 and DLD-1 cells carrying the KRAS G13D mutation were employed as a control. Cells were serum-starved for 48 h and then lysed. 2 mg of whole-cell cleared lysate was incubated with 35 μg of GST-RAF CRIB for 30 min at 4° C. The complexes were collected by centrifugation and washed three times with lysis buffer. Proteins were separated by SDS page, followed by Western blot. The kras protein was detected with Anti-Pan-Ras (Ab-3) mAb (Oncogene, Calbiochem, San Diego, Calif.). Signal was developed using the ECL system (Amersham Biosciences, Uppsala, Sweden).

### Proliferation Assay

WT and KI hTERT-HME1 cells (4×10³) were seeded in triplicates in 96-well plates in complete medium (10% serum, EGF and insulin containing medium) at equal density on day 0 and cell number was measured every 24 h for 7 days by a luminescence ATP assay (ATPlite 1 step kit, Perkin Elmer, Milan, Italy). All luminescence measurements (indicated as relative light units, RLUs) were recorded by the DTX 880-Multimode plate reader (Beckman-Coulter).

### Soft Agar Anchorage-Independent Growth Assay

To assess anchorage-independent growth, 5×10⁵ cells were mixed 10:1 with 5% agarose in complete growth medium, for a final concentration of 0.5% agarose. The cell mixture was plated on top of a solidified layer of 1% agarose-growth medium in 12-well plates. Cells were supplemented every 2-3 days with 200 μl of growth complete medium. Cells were stained with 0.02% iodonitrotetrazolium chloride (Sigma-Aldrich, St. Louis, Mo.) and photographed after 14 days. Images were captured with the ImageReady software (Adobe) using a microscope (DMIL; Leica) equipped with a digital camera (DFC320; Leica).

### Chemicals and Drugs

Chemicals and drugs were purchased from several different commercial suppliers as indicated in table 3. All compounds were reconstituted in the appropriate solvents and stored in aliquots at the temperature recommended by the manufacturers.

The chemicals and drugs indicated in table 3 can be grouped in the following categories: chemotherapeutic agents, tyrosine kinase inhibitors, anti-proliferative agents, antiemetics, antacids, H2 antagonists, proton pump inhibitors, laxatives, anti-obesity drugs, anti-diabetics, vitamins, dietary minerals, antithrombotics, antihemorrhagics, antianginals, antihypertensives, diuretics, vasolidators, beta blockers, calcium channel blockers, rennin-angiotensin system drugs, antihyperlipidemics (statins, fibrates, bile acid sequestrants), antipsoriatic, sex hormones, hormonal contraceptives, fertility agents, SERMs, hypothalamic-pituitary hormones, corticosteroids (glucocorticoids, mineralocorticoids), thyroid hormones/antithyroid agents, antibiotics, antifungals, antimycobacterial, antivirals, vaccines, antiparasitic (antiprotozoals, anthelmintics), immunomodulators (immunostimulators, immunosuppressants), anabolic steroids, anti-inflammatories (NSAID), antirheumatics, corticosteroids, muscle relaxants, bisphosphonate, anesthetics, analgesics, antimigraines, anticonvulsants, mood stabilizers, antiparkinson drug, psycholeptic (anxiolytics, antipsychotics, hypnotics/sedatives), psychoanaleptic (antidepressants, stimulants/psychostimulants), decongestants, bronchodilators, H1 antagonists.

TABLE 3

| | | | | Drug | |
|---|---|---|---|---|---|
| | | | | concentration | |
| | | | Catalog | Log [M] | |
| OGC ID | Compound | Company | number | min | max |
| OGC-001 | 8-Allylnaringenin | CS | | −4.95 | −3.74 |
| OGC-002 | Apigenin | CS | | −5.48 | −4.52 |
| OGC-003 | Artemetin | CS | | −5.12 | −3.92 |
| OGC-004 | Degueline | CS | | −7.40 | −4.10 |
| OGC-005 | Erybraedin C | CS | | −5.30 | −4.70 |
| OGC-006 | 8-Geranylapigenin | CS | | −5.40 | −4.44 |
| OGC-007 | 8- | CS | | −5.30 | 3.44 |
| | Geranylnaringenin | | | | |
| OGC-008 | Eupatiline | CS | | −5.30 | −3.97 |
| OGC-009 | Genistein | CS | | −5.30 | −4.10 |
| OGC-010 | Isosakuranetin | CS | | −5.00 | −3.52 |
| OGC-011 | Naringenin | CS | | −4.22 | −3.05 |
| OGC-012 | 8-Prenylapigenin | CS | | −6.00 | −3.74 |
| OGC-013 | 8-Prenylnaringenin | CS | | −4.52 | −3.05 |
| OGC-014 | 8-Prenylgenistein | CS | | −5.52 | −4.12 |
| OGC-015 | 8-Prenylquercetin | CS | | −5.48 | −4.14 |
| OGC-016 | Pre-rotenone | CS | | −6.30 | −4.30 |
| OGC-017 | Quercetin | CS | | −5.00 | −3.92 |
| OGC-018 | Rotenone | CS | | −7.10 | −5.00 |
| OGC-019 | Sakuranetin | CS | | −4.52 | −3.57 |
| OGC-020 | LY 294002 | Calbiochem | 440202 | −5.80 | −4.40 |
| OGC-021 | LY 303511 | Alexis | ALX-270- | −5.22 | −4.05 |
| | | | 410 | | |
| OGC-022 | Wortmannin | Alexis | ALX-350- | −5.10 | −3.70 |
| | | | 020 | | |
| OGC-023 | 1L6-Hydroxymethyl- | Alexis | ALX-270- | −5.00 | −4.05 |
| | chiro-inositol-2- | | 292 | | |
| | (R)-2-O-methyl-3- | | | | |
| | O-octadecyl-sn- | | | | |
| | glycerocarbonate | | | | |
| OGC-024 | Triciribine. Akt | Calbiochem | 124005 | −8.70 | −4.70 |
| | Inhibitor V | | | | |
| OGC-025 | PD 98059 | Calbiochem | 513001 | −4.52 | −3.57 |
| OGC-026 | U0126 | Promega | V1121 | −5.22 | −3.60 |
| OGC-027 | Rapamycin | Alexis | ALX-380- | −11.00 | −6.00 |
| | | | 004 | | |
| OGC-028 | Tamoxifen 4- | Calbiochem | 579002 | −5.12 | −4.52 |
| | Hydroxy-(Z) | | | | |
| OGC-029 | Bisindolylmaleimide I | Alexis | ALX-270-049 | −5.70 | −4.70 |
| OGC-030 | SU11274 | Calbiochem | 448101 | −5.55 | −5.20 |
| OGC-031 | Gefitinib | SRP | SRP01240g | −7.30 | −4.44 |
| OGC-032 | Erlotinib mesylate | SRP | SRP01330e | −7.30 | −4.40 |
| OGC-033 | Imatinib mesylate | SRP | SRP00530i | −5.52 | −4.44 |
| OGC-034 | Sunitinib Maleate | SRP | SRP01785s | −5.78 | −4.74 |
| OGC-035 | Sorafenib Tosylate | SRP | SRP01590s | −5.95 | −5.00 |
| OGC-036 | Cetuximab | HP | | −7.65 | −5.39 |
| OGC-037 | Acetylsalicylic | Sigma- | 239631 | −3.10 | −2.14 |
| | Acid | Aldrich | | | |
| OGC-038 | Sodium Salicylate | Sigma- | 241350 | −4.00 | −1.87 |
| | | Aldrich | | | |
| OGC-039 | Mesalazine | Sigma- | A3537 | −3.65 | −1.27 |
| | | Aldrich | | | |
| OGC-040 | Paracetamol | Sigma- | A5000 | −3.52 | −2.14 |
| | | Aldrich | | | |
| OGC-041 | Meloxicam | HP | | −4.40 | −3.05 |
| OGC-042 | Celecoxib | HP | | −5.10 | −3.35 |
| OGC-043 | Rofecoxib | SRP | SRP013045r | −4.60 | −3.27 |
| OGC-045 | Indomethacin | HP | | −4.70 | −3.40 |
| OGC-046 | Nimesulide | Sigma- | N1016 | −4.30 | −3.22 |
| | | Aldrich | | | |
| OGC-047 | Diclofenac | HP | | −4.70 | −3.74 |
| OGC-048 | Ondansetron | HP | | −4.30 | −3.70 |
| OGC-049 | Cimetidine | HP | | −3.22 | −1.97 |
| OGC-050 | Ranitidine | HP | | −3.40 | −2.44 |
| OGC-051 | Omeprazole | HP | | −4.40 | −3.40 |
| OGC-052 | Metoclopramide | HP | | −4.52 | −3.57 |
| OGC-053 | Procainamide | Sigma- | P9391 | −3.40 | −2.40 |
| | | Aldrich | | | |
| OGC-054 | Sodium | Calbiochem | 567616 | −3.52 | −1.97 |
| | Phenylbutyrate | | | | |
| OGC-055 | Ergocalciferol | HP | | −6.05 | −5.09 |
| OGC-056 | Calcitriol | HP | | −5.40 | −3.74 |
| OGC-057 | Simvastatin | SRP | SRPO1380s | −6.48 | −5.49 |
| OGC-058 | Lovastatin | SRP | SRPO1585l | −6.52 | −5.27 |
| OGC-059 | Atorvastatin Ca | SRP | SRPO7330a | −6.52 | −5.27 |
| OGC-060 | Fluvastatin Na | SRP | SRPO1980f | −7.12 | −5.57 |
| OGC-061 | Pravastatin Na | SRP | SRPO2590p | −5.52 | −4.27 |
| OGC-062 | Tamoxifene Citrate | Calbiochem | 579000 | −5.70 | −4.74 |
| OGC-063 | Raloxifene | Sigma- | R1402 | −5.70 | −4.74 |
| | Hydrochloride | Aldrich | | | |
| OGC-064 | Fulvestrant | HP | | −5.00 | −4.05 |
| OGC-066 | Erythromycin | Sigma- | 45673-5G-F | −4.30 | −3.10 |
| | | Aldrich | | | |
| OGC-067 | Clodronic Acid | HP | | −3.70 | −2.44 |
| OGC-068 | Zoledronic Acid | HP | | −5.70 | −4.70 |
| OGC-069 | Estradiol | HP | | −4.48 | −3.52 |
| OGC-070 | Paclitaxel | HP | | −10.70 | −7.00 |
| OGC-071 | Mevastatin | SRP | SRPO6551m | −6.60 | −5.05 |
| OGC-072 | Itavastatin Ca | SRP | SRPO2390i | −7.60 | −5.74 |
| OGC-073 | Rosuvastatin Ca | SRP | SRPO1326r | −5.52 | −4.27 |
| OGC-074 | Everolimus | Sigma- | 7741 | −9.30 | −4.70 |
| | | Aldrich | | | |
| OGC-075 | Dasatinib | SRP | SRP09030d | −8.60 | −5.40 |
| | monohydrate | | | | |
| OGC-076 | Compound C | Sigma- | P5499 | −5.70 | −4.49 |
| | | Aldrich | | | |
| OGC-077 | Rimonabant | SRP | SRP01287r | −5.30 | −4.22 |
| OGC-078 | Anandamide | Cayman | CAY-90050 | −4.70 | −3.57 |
| OGC-079 | Met-F-AEA | Cayman | CAY-90055 | −4.70 | −3.74 |
| OGC-080 | JWH-015 | Cayman | CAY-10009018 | −4.70 | −3.55 |
| OGC-081 | 17-Allylamino | Alexis | ALX380-091 | −7.70 | −6.00 |
| | geldanamycin | | | | |
| OGC-082 | Doxorubicin | HP | | −9.00 | −5.00 |
| | hydrochloride | | | | |
| OGC-083 | 5-FU | HP | | −5.82 | −3.52 |
| OGC-084 | Cisplatin | HP | | −6.70 | −4.30 |
| OGC-085 | Sulindac | Cayman | CAY-10004386 | −4.20 | −3.00 |
| OGC-086 | Sulindac sulfide | Alexix | ALX-430-106 | −4.52 | −3.85 |
| OGC-087 | 17-DMAG | Alexis | ALX380-110 | −8.40 | −7.00 |
| OGC-088 | Trastuzumab | HP | | −6.70 | −4.27 |
| OGC-089 | THC | CS | | −5.30 | −3.40 |
| OGC-090 | Parthenolide | CS | | −6.35 | −5.22 |
| OGC-091 | Pseudolaric Acid B | CS | | −6.90 | −5.70 |
| OGC-092 | Irinotecan | HP | | −6.52 | −4.52 |
| OGC-093 | Vinorelbine | HP | | −9.40 | −7.30 |
| OGC-095 | IMMA (BML-190) | Cayman | CAY-70275 | −4.48 | −3.30 |
| OGC-096 | AM404 | Alexis | ALX-340-032 | −4.70 | −4.10 |
| OGC-097 | PI-103 | Cayman | CAY-10009209 | −8.00 | −6 |
| OGC-098 | ZSTK404 | Alexis | ALX-270-454 | −6.70 | −4.7 |
| OGC-126 | CI-1040 (PD | Alexis | ALX-270-471 | −6.81 | −4.7 |
| | 184352) | | | | |

Abbreviations:
CS: Custom Synthesis;
SRP: Sequoia Research Products;
HP: Hospital Pharmacy

### Plasmids and Viral Vectors

All the KI targeting vectors were constructed using a modified pBluescript plasmid, which was named pSA-5A, containing a Neo resistance gene driven by a SV40 promoter; two loxP sites flank this G418 resistance cassette (SEQ ID No.: 8 to 14). The list of primers employed to amplify the homology arms is available in table 4. All experimental procedures for targeting vector construction, AAV production, cell infection and screening for recombinants have already been described in (7). The list of primers used for screening is provided in table 5. The lentiviral vector expressing BRAF V600E was a kind gift of Dr. Maria S. Soengas from the University of Michigan as described in M. Verhaegen, et al. 2006 (3). The procedure to obtain the lentivirus expressing the KRAS G13D mutation has been described in (1).

TABLE 4

| | AA | Homol. | gDNA | Primers | Restriction |
|---|---|---|---|---|---|
| Gene | Mutation | arm | source | (F = forward; R = reverse) | sites |
| BRAF | V600E | 5′ | HT-29 | F | Eco RI, |
| | | | | tgaaaaGAATTCGCGGCCGCataac | NotI, loxP |
| | | | | ttcgtataatgtatgctatacgaag | |
| | | | | ttatgttttcatgctaagttcgat | |
| | | | | SEQ ID No.: 15 | |
| | | | | R | Eco RI |
| | | | | aaataaGAATTCtgatttttgtgaa | |
| | | | | tactgggaac | |
| | | | | SEQ ID No.: 16 | |
| | | 3′ | hTERT RPE-1 | F | Xba I |
| | | | | tcacaaTCTAGAgtgttcttatttt | |
| | | | | ttatgta | |
| | | | | SEQ ID No.: 17 | |
| | | | | R | Xba I |
| | | | | ctcactTCTAGAagcaggccagtca | |
| | | | | actcct | |
| | | | | SEQ ID No.: 18 | |
| CTNNB1 | T41A | 5′ | GenScript* | F | Eco RV, Not I, |
| | | | | ATATCaGCGGCCGCagaattcGTT | EcoRI |
| | | | | GCCATTAAGCCAGTCTG | |
| | | | | SEQ ID No.: 77 | |
| | | | | R | Eco RV, |
| | | | | GATATCGAATTCTTTTATTTAAACT | EcoRI |
| | | | | ATTATAC | |
| | | | | SEQ ID No.: 78 | |
| | | 3′ | hTERT-HME1 | F | Xba I, Spe |
| | | | | ATAATATCTAGAACTAGTTGTTGTG | I |
| | | | | GTGAAGAAAAGAGAG | |
| | | | | SEQ ID No.: 79 | |
| | | | | R | Xba I, Spe |
| | | | | GAATCTTCTAGAACTAGTTCTGAGG | I |
| | | | | TGGAATGGTGTCA | |
| | | | | SEQ ID No.: 80 | |
| EGFR | de1E746- | 5′ | GenScript* | F | Eco RI, |
| | A750 | | | ggaaatGAATTCGCGGCCGCataac | NotI, loxP |
| | | | | ttcgtataatgtatgctatacgaag | |
| | | | | ttatatcagtggtcctgtgag | |
| | | | | SEQ ID No.: 19 | |
| | | | | R | Eco RI |
| | | | | cccactGAATTCagaaagggaaaga | |
| | | | | catagaaa | |
| | | | | SEQ ID No.: 20 | |
| | | 3′ | hTERT-RPE1 | F | Nhe I |
| | | | | ctttccGCTAGCagctctagtgggt | |
| | | | | ataactccc | |
| | | | | SEQ ID No.: 21 | |
| | | | | R | Nhe I |
| | | | | tacacaGCTAGCgtgaggggccaga | |
| | | | | gattgta | |
| | | | | SEQ ID No.: 22 | |
| KRAS | G13D | 5′ | hTERT-RPE1 | F | Eco RI, Not I |
| | | | | taggcgGAATTCGCGGCCGCcggct | |
| | | | | cacttgcatctctta | |
| | | | | SEQ ID No.: 23 | |
| | | | | R | Eco RI |
| | | | | tgactgGAATTCtgtatcgtaatga | |
| | | | | actgtacttc | |
| | | | | SEQ ID No.: 24 | |
| | | 3′ | DLD1 | F | Xba I |
| | | | | cattacTCTAGAcgtctgcagtcaa | |
| | | | | ctggaat | |
| | | | | SEQ ID No.: 25 | |
| | | | | R | Xba I, loxP |
| | | | | gacagtTCTAGAataacttcgtata | |
| | | | | gcatacattatacgaagttatatat | |
| | | | | cctcatctgcttgggatg | |
| | | | | SEQ ID No.: 26 | |
| PIK3CA | E545K | 5′ | hTERT-RPE1 | F | Eco RV, Not I |
| | | | | ttatttGATATCGCGGCCGCaggct | |
| | | | | tgcagtgttttctcc | |
| | | | | SEQ ID No.: 27 | |
| | | | | R | Eco RV |
| | | | | ctggatGATATCatgatttacagaa | |
| | | | | aaagcaa | |
| | | | | SEQ ID No.: 28 | |
| | | 3′ | ME180 | F | Spe I |
| | | | | tctgtaACTAGTctgtgaatccaga | |
| | | | | ggggaaa | |
| | | | | SEQ ID No.: 29 | |
| | | | | R | Spe I |
| | | | | gcacagACTAGTtggcaaagaacac | |
| | | | | aaaagga | |
| | | | | SEQ ID No.: 30 | |
| | H1047R | 5′ | HCT116 | F | Eco RI, |
| | | | | ggtttcGAATTCGCGGCCGCgctgg | NotI |
| | | | | tcttgaactcccaa | |
| | | | | SEQ ID No.: 31 | |
| | | | | R | Eco RI |
| | | | | ttggagGAATTCatgttaatacctt | |
| | | | | caggtctttgc | |
| | | | | SEQ ID No.: 32 | |
| | | 3′ | HCT116 | F | Xba I |
| | | | | aggtatTCTAGAcatttgctccaaa | |
| | | | | ctgacca | |
| | | | | SEQ ID No.: 33 | |
| | | | | R | Xba I, loxP |
| | | | | tgtccaTCTAGAataacttcgtata | |
| | | | | atgtatgctatacgaagttatGTGA | |
| | | | | CTGCTTCCAAAACTGC | |
| | | | | SEQ ID No.: 34 | |

| | | |
|---|---|---|
| PTEN | R130* | THE ENTIRE CASSETTE Not I-PTEN-Not I was completely custom- |
| | | synthesized by Genescritpt |

TABLE 5

| | AA | | Primers | |
|---|---|---|---|---|
| Gene | Mutation | Exon | (F = forward; R = reverse) | Sequence primer |
| BRAF | V600E | 15 | F | TGTTTTCCTTTACTTACTACACC |
| | | | CCTGAAATTTGTCTGCGAAGT | TCA |
| | | | SEQ ID No.: 35 | SEQ ID No.: 39 |
| | | | R | |
| | | | TCTTTCCGCCTCAGAAGGTA | |
| | | | SEQ ID No.: 36 | |
| | | | F | |
| | | | CCTGAAATTTGTCTGCGAAGT | |
| | | | SEQ ID No.: 37 | |
| | | | R | |
| | | | TGATTTTTGTGAATACTGGGAAC | |
| | | | SEQ ID No.: 38 | |
| EGFR | de1E746- | 19 | F | GCTGGTAACATCCACCCAGA |
| | A750 | | GCTGAGGTGACCCTTGTCTC | SEQ ID No.: 44 |
| | | | SEQ ID No.: 40 | |
| | | | R | |
| | | | GCTTGGCTGGACGTAAACTC | |
| | | | SEQ ID No.: 41 | |
| | | | F | |
| | | | GCTGAGGTGACCCTTGTCTC | |
| | | | SEQ ID No.: 42 | |
| | | | R | |
| | | | CCACACAGCAAAGCAGAAAC | |
| | | | SEQ ID No.: 43 | |
| KRAS | G13D | 2 | F | GGTGGAGTATTTGATAGTGTATT |
| | | | GCCTTCTATCGCCTTCTTGA | AACC |
| | | | SEQ ID No.: 45 | SEQ ID No.: 51 |
| | | | R | |
| | | | ACAAGGACAGTTGGGGAATG | |
| | | | SEQ ID No.: 46 | |
| | | | F | |
| | | | TCTTGACGAGTTCTTCTGAGC | |
| | | | SEQ ID No.: 47 | |
| | | | R | |
| | | | AACAAGGACAGTTGGGGAAT | |
| | | | SEQ ID No.: 48 | |
| | | | F | |
| | | | CGTCTGCAGTCAACTGGAAT | |
| | | | SEQ ID No.: 49 | |
| | | | R | |
| | | | AACAAGGACAGTTGGGGAAT | |
| | | | SEQ ID No.: 50 | |
| PIK3CA | E545K | 9 | F | GGGAAAAATATGACAAAGAAAGC |
| | | | AGGACATAGCGTTGGCTACC | SEQ ID No.: 60 |
| | | | SEQ ID No.: 52 | |
| | | | R | |
| | | | TGGGTAGAATTTCGGGGATA | |
| | | | SEQ ID No.: 53 | |
| | | | F | |
| | | | CTGTGAATCCAGAGGGGAAA | |
| | | | SEQ ID No.: 54 | |
| | | | R | |
| | | | TGGGTAGAATTTCGGGGATA | |
| | | | SEQ ID No.: 55 | |
| | | | F | |
| | H1047R | 20 | TCTTGACGAGTTCTTCTGAGC | |
| | | | SEQ ID No.: 56 | |
| | | | R | |
| | | | TTGTGGGAGCCCAGAATTT | |
| | | | SEQ ID No.: 57 | |
| | | | F | |
| | | | CATTTGCTCCAAACTGACCA | |
| | | | SEQ ID No.: 58 | |
| | | | R | |
| | | | TTGTGGGAGCCCAGAATTT | |
| | | | SEQ ID No.: 59 | |
| PTEN | R130* | 5 | F | TCCAGGAAGAGGAAAGGAAAA |
| ZEO | | | GCTGCAGTCCATTGAGCATA | SEQ ID No.: 69 |
| | | | SEQ ID No.: 61 | |
| | | | R | |
| | | | TCCAGGAAGAGGAAAGGAAAA | |
| | | | SEQ ID No.: 62 | |
| | | | F | |
| | | | GCTGCAGTCCATTGAGCATA | |
| | | | SEQ ID No.: 63 | |
| | | | R | |
| | | | GCTTGGCTGGACGTAAACTC | |
| | | | SEQ ID No.: 64 | |
| | | | F | |
| | | | GCTGCAGTCCATTGAGCATA | |
| PTEN | R130* | 5 | SEQ ID No.: 65 | |
| NEO | | | R | |
| | | | TCCAGGAAGAGGAAAGGAAAA | |
| | | | SEQ ID No.: 66 | |
| | | | F | |
| | | | GCTGCAGTCCATTGAGCATA | |
| | | | SEQ ID No.: 67 | |
| | | | R | |
| | | | TCTTTCCGCCTCAGAAGGTA | |
| | | | SEQ ID No.: 68 | |
| CTNNB1 | T41A | 3 | F | CAGGACTTGGGAGGTATCCA |
| | | | GATGGAGCTGTGGTTGAGGT | SEQ ID No.: 74 |
| | | | SEQ ID No.: 70 | |
| | | | R | |
| | | | TCAAAACTGCATTCTGACTTTCA | |
| | | | SEQ ID No.: 71 | |
| | | | F | |
| | | | GATGGAGCTGTGGTTGAGGT | |
| | | | SEQ ID No.: 72 | |
| | | | R | |
| | | | TCTTTCCGCCTCAGAAGGTA | |
| | | | SEQ ID No.: 73 | |

RNA Extraction and cDNA Synthesis

To confirm the expression of the mutation at the transcriptional level, total RNA was isolated using the SV Total RNA Isolation System kit (Promega, Madison, Wis.) and reverse transcribed as previously described (1). 2 μL of the corresponding cDNA were directly amplified using Taq DNA Polymerase-mediated PCR reactions. A forward primer and a reverse primer annealing on the homology arm containing each mutation of the different constructs were used to produce the amplicon containing the mutated expressed sequence. The amplicons were sequenced to verify the expression of the introduced mutation at the RNA level.

### Cre-Mediated Excision of Selectable Marker Elements and PCR Analysis

To remove the Neo cassette from correctly targeted clones, cells were infected with an adenovirus that expresses the Cre recombinase. 24 h after infection, cells were plated in 96-well plates at limiting dilution using a non-selective medium. After 2 weeks, when cells in 96-well plates reached ˜60-80% confluence, DNA was extracted from single clones using Lyse-N-G0™ PCR Reagent (Pierce, Rockford, Ill.), as described above. The Neo cassette removal was assessed by PCR, as already described elsewhere (7). The presence of the targeted alleles was further reconfirmed by sequencing. To obtain DKI clones carrying both PIK3CA and EGFR mutations, a heterozygous PIK3CA KI clone (from which the Neo cassette was removed) was infected with the EGFR KI rAAV virus.

### Pharmacology Data Analysis (Pharmarray)

Cell growth inhibition at each drug concentration was initially normalized to vehicle treated cells for each clone. Then within each experiment we calculated a parameter that we named ‘Δ knock-in’ (ΔKI), corresponding to the variation expressed e.g. as percentage of inhibition between a KI clone and its parental line at each compound concentration and its corresponding signal to noise ratio (SNR)

SNR=|ΔKI|/{√[σ(WT)²+σ(KI)²]}.

To be considered significantly ‘KI specific’ at a given concentration in one experiment, a compound had to simultaneously display a |ΔKI|>30 and a SNR>10. A minimum of three experiments for each cell line were then summarized by calculating the average and standard deviation of the ΔKI values, and finally the averaged ΔKI values were included in the final report only when they were greater than 2σ and were significant in at least one experiment; we also included in the final analysis averaged ΔKI values that were greater than 3σ despite not being significant in any single experiment. All other ΔKI values not satisfying the stringent statistical criteria above mentioned were assigned a final ‘0’ score. All the analyzed ΔKI values were visualized using a recently developed gene expression data analysis program, named GEDAS (8). To allow a direct visualization of the different color shades, all ΔKI values were scaled down 5-fold. In fact, the maximum and minimum theoretical ΔKI values calculated by our method would be +100 (in case of a compound concentration killing 100% KI cells with no effect on the parental line) and −100 (in case of a compound concentration not affecting KI cells while killing all WT cells), respectively, while the GEDAS software allows visualization of data with a maximum fold change of ±20.

### Statistics

The NOEL (highest no observed effect level), IC₅₀ and IC₉₀ values for each drug were calculated using GraphPad Prism 4.0 software. Where indicated the results are given as the mean±s.d. Statistical analyses were performed by the two-tailed t-test with Bonferroni's multiple comparisons correction using the Instat program (GraphPad, GraphPad Software, Inc. San Diego, Calif.). Differences of means were considered significant at a significance level of 0.05 (*: p<0.05; **: p<0.01; ***: p<0.001).

### Results

### KI of Mutated BRAF, CTNNB1, PTEN, EGFR, ERAS and PIK3CA Alleles in the Genome of Human Cells

AAV mediated homologous recombination was employed to introduce somatic mutations commonly found in tumors in human somatic cells. Specifically the inventors focused on the following alleles EGFR (delE746-A750), KRAS (G13D), BRAF (V600E), CTNNB1 (T41A), PTEN (R130*) and PIK3CA (E545K and H1047R) that are found in multiple cancer types. These include among others lung (EGFR and KRAS), colorectal (KRAS, CTNNB1, BRAF, PIK3CA), breast (PIK3CA and PTEN), pancreatic (KRAS) and prostate (KRAS, BRAF and PTEN) carcinomas and melanoma (BRAF).

As recipient cells three non-transformed epithelial cell lines of breast (MCF10A, hTERT-HME1) and retinal (hTERT RPE-1) origin, and one cancer cell line (SW48) derived from a colorectal carcinoma were employed. These cells display a number of features rendering them appealing for genetic and biological manipulation. The cells derived from the breast and retinal epithelium can be propagated indefinitely in vitro, but are not tumorigenic, which makes them a suitable model to study oncogene-mediated transformation. Furthermore, these three cell lines have been previously used to assess a number of cellular phenotypes including growth factor dependent proliferation, motility and invasive growth. The colorectal cancer cell line SW48 was selected because (despite being fully tumorigenic) it does not carry any of the above-mentioned alleles and was therefore suitable as a recipient test platform for the KI approaches.

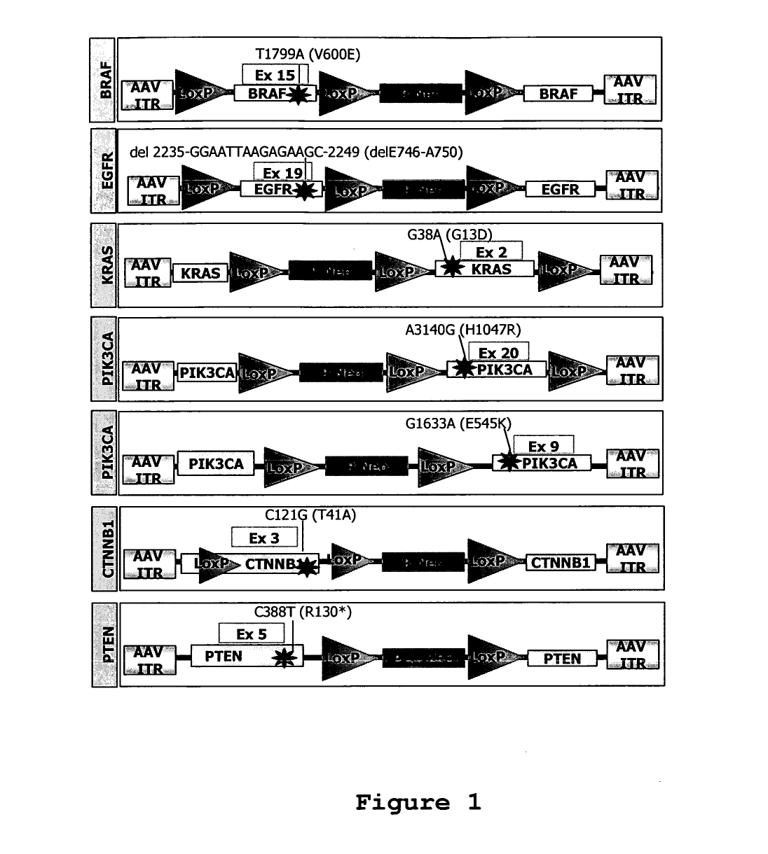

A common strategy was used to generate the recombinant AAV vectors required to knock-in each of the six cancer alleles (FIG. 1). In brief, the homologous recombination cassette was cloned within the AAV ITRs and consisted of two ˜1 kb sequences ('homology arms'), one of which contained the specific mutation (PI3KCA mutated homology arms are shown in SEQ ID No.:11 and 12, BRAF mutated homology arm is shown in SEQ ID No.:9, KRAS mutated homology arm is shown in SEQ ID No.:10, EGFR mutated homology arm is shown in SEQ ID No.:14, CTNNB1 mutated homology arm is shown in SEQ ID No.:8, and PTEN mutated homology arm is shown in SEQ ID No.:13). A selectable marker (SEQ ID NO.:75 and SEQ ID NO.:76) was placed between the homology arms flanked by two LoxP sites, to allow Cre recombinase mediated excision of the Neo cassette from the genome of the targeted cells (FIG. 1).

After infection with rAAV and G418 selection, clones with locus-specific integration of the targeted alleles were identified through a PCR screening approach as disclosed above. Positive clones were expanded and gDNA and RNA were extracted in order to sequence the targeted region to independently confirm the presence and the expression of the specific mutations.

Double KI clones carrying both the PIK3CA (H1047R) and EGFR (delE746-A750) mutations (hereafter referred to as DKI) were also generated in MCF10A and hTERT-HME1 cells, starting from clones in which the PIK3CA (H1047R) alteration had already been introduced. After infection with an adenovirus expressing the Cre recombinase to remove the Neo cassette, the PIK3CA Cre-out KI clones were infected with the EGFR-rAAV. Identification of the EGFR (delE746-A750) targeted clones was achieved as described for the single KI approach.

Following a similar experimental approach, other DKI clones carrying respectively KRAS (G13D) and PIK3CA (H1047R), EGFR (delE746-A750) and BRAF (V600E) were also generated. To account for clonal variability, multiple independent cell lines carrying each of the mutations were generated and analyzed at the biochemical, biological and pharmacological levels.

### Biochemical Analysis of Mutated Alleles in Human Cells

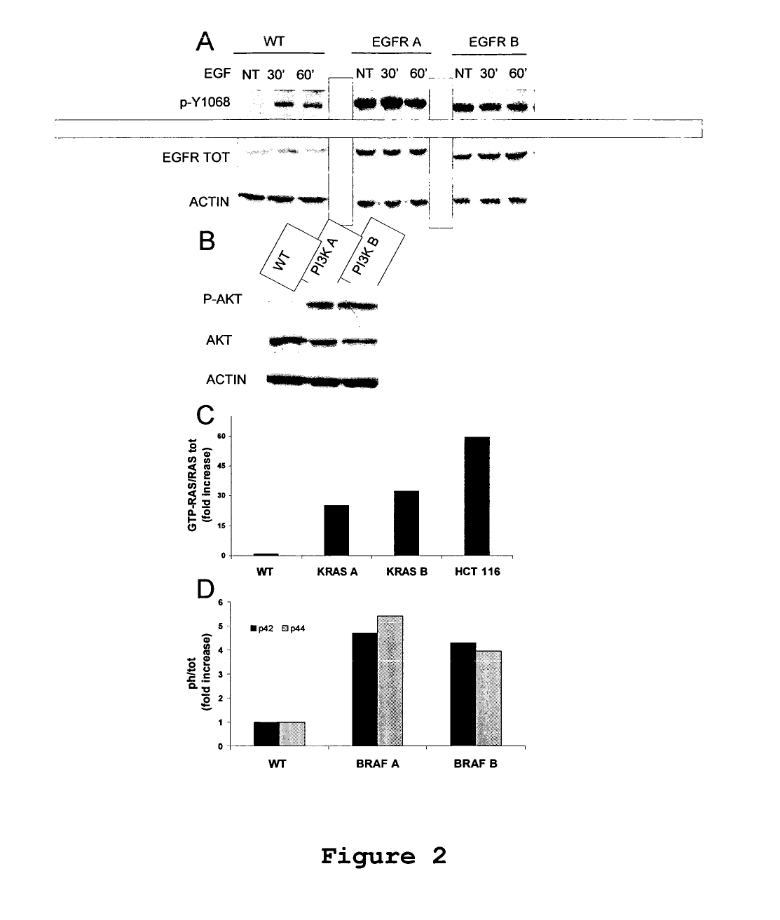

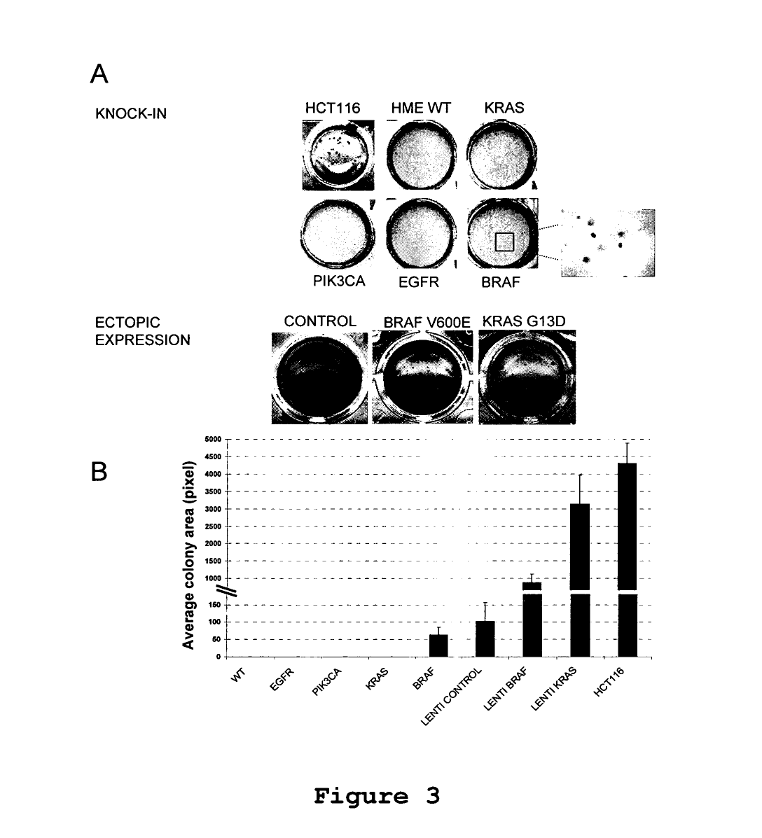

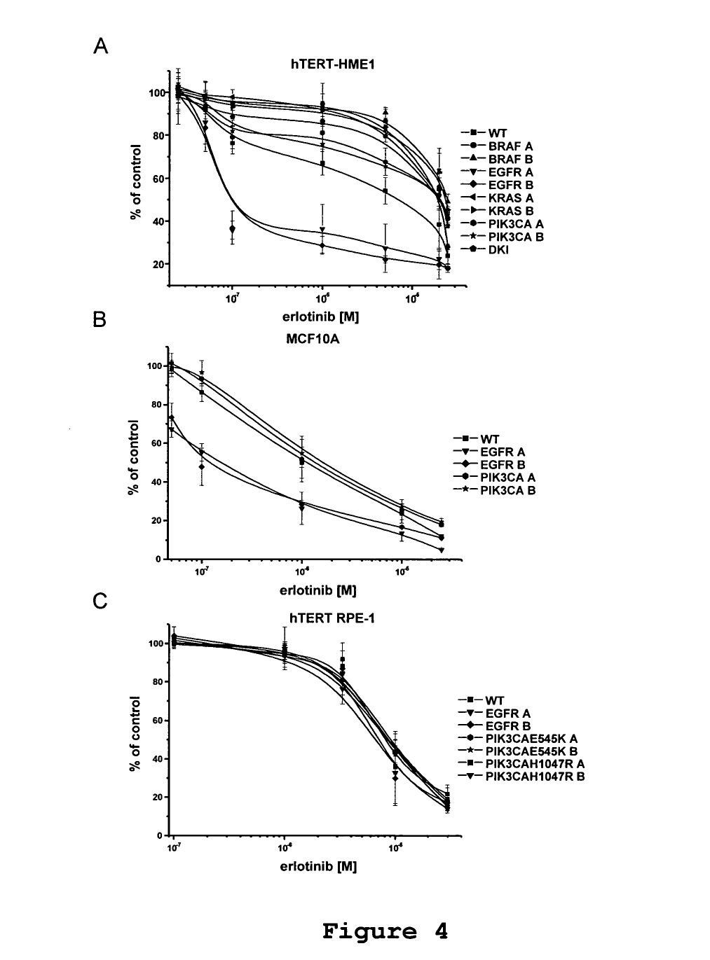

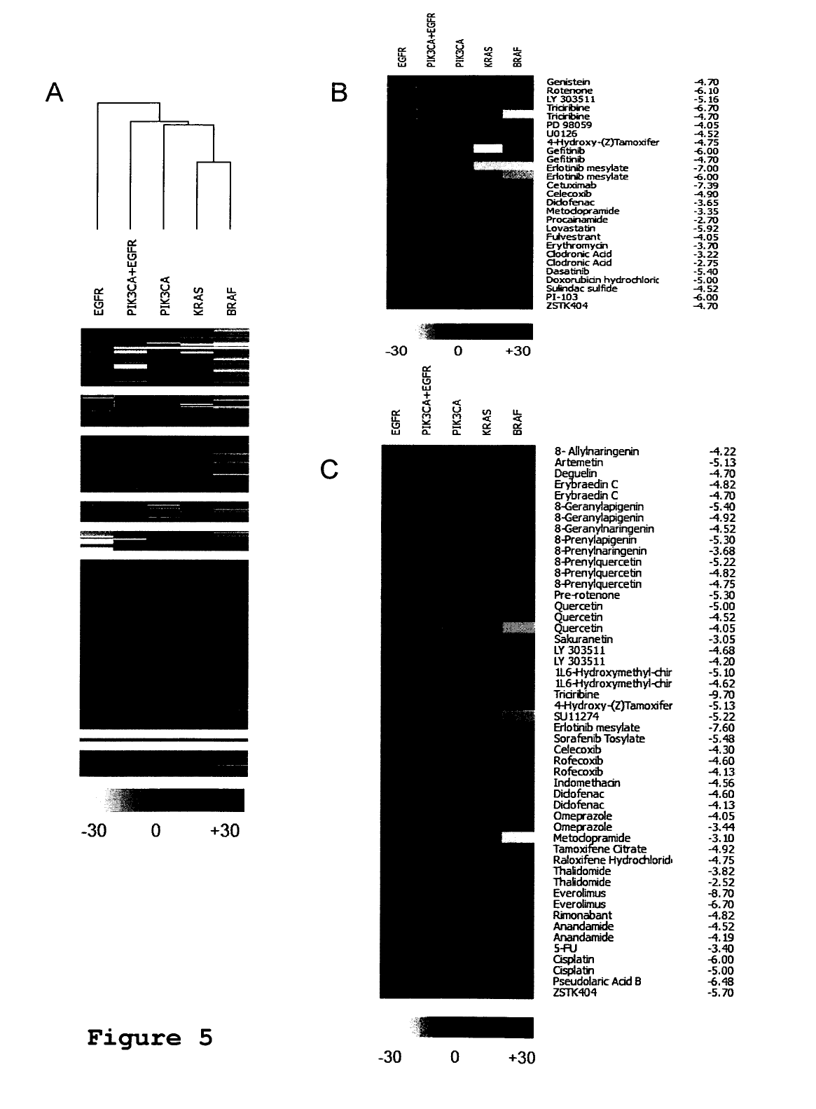

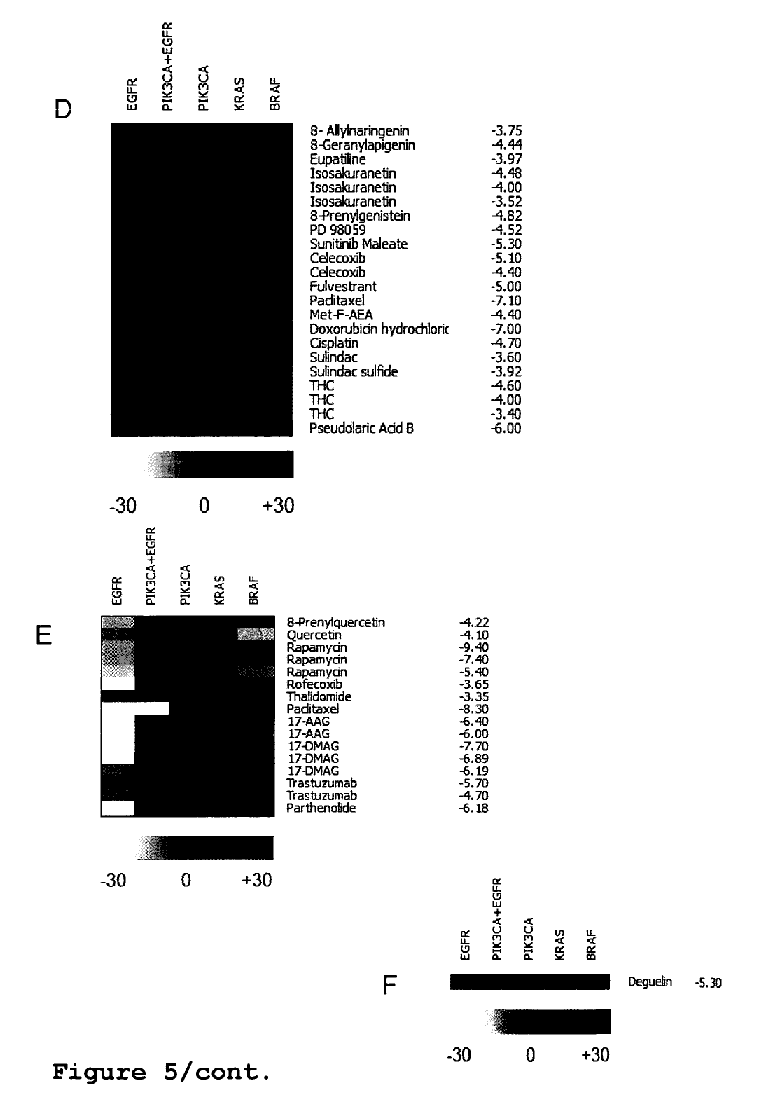

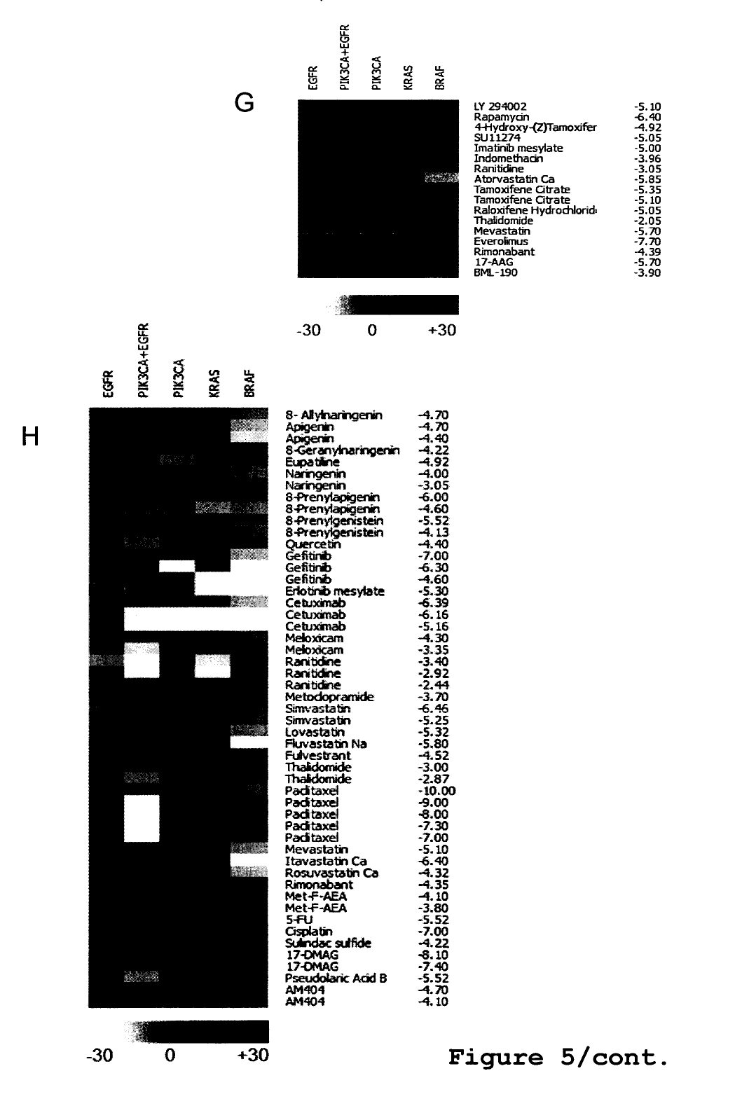

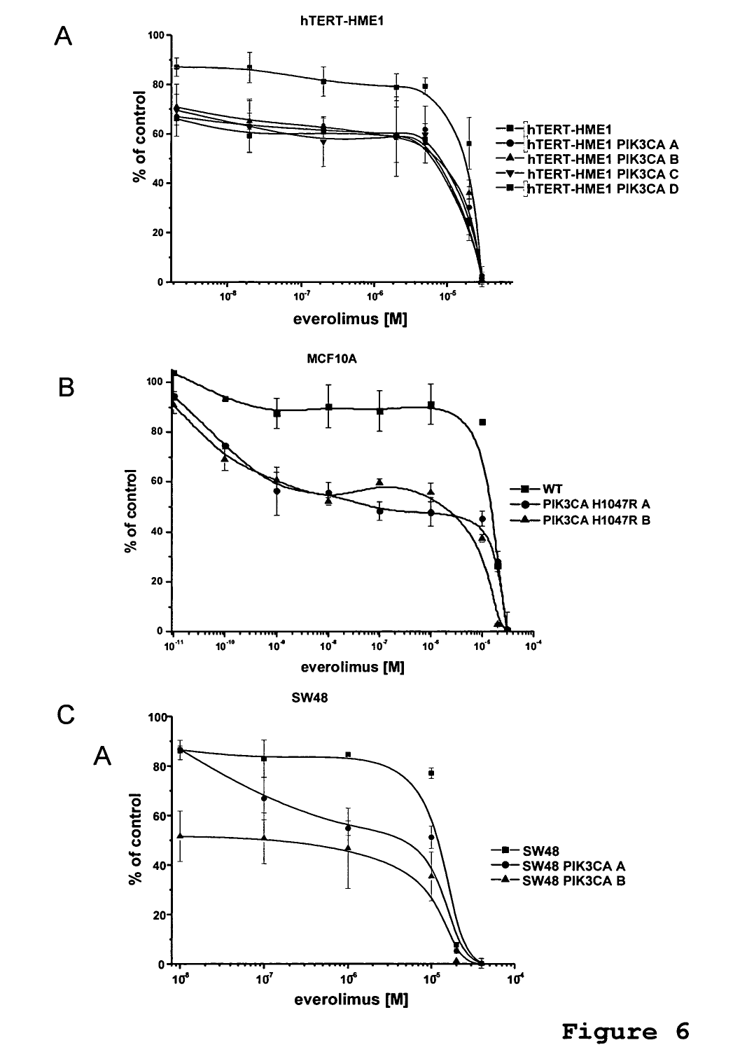

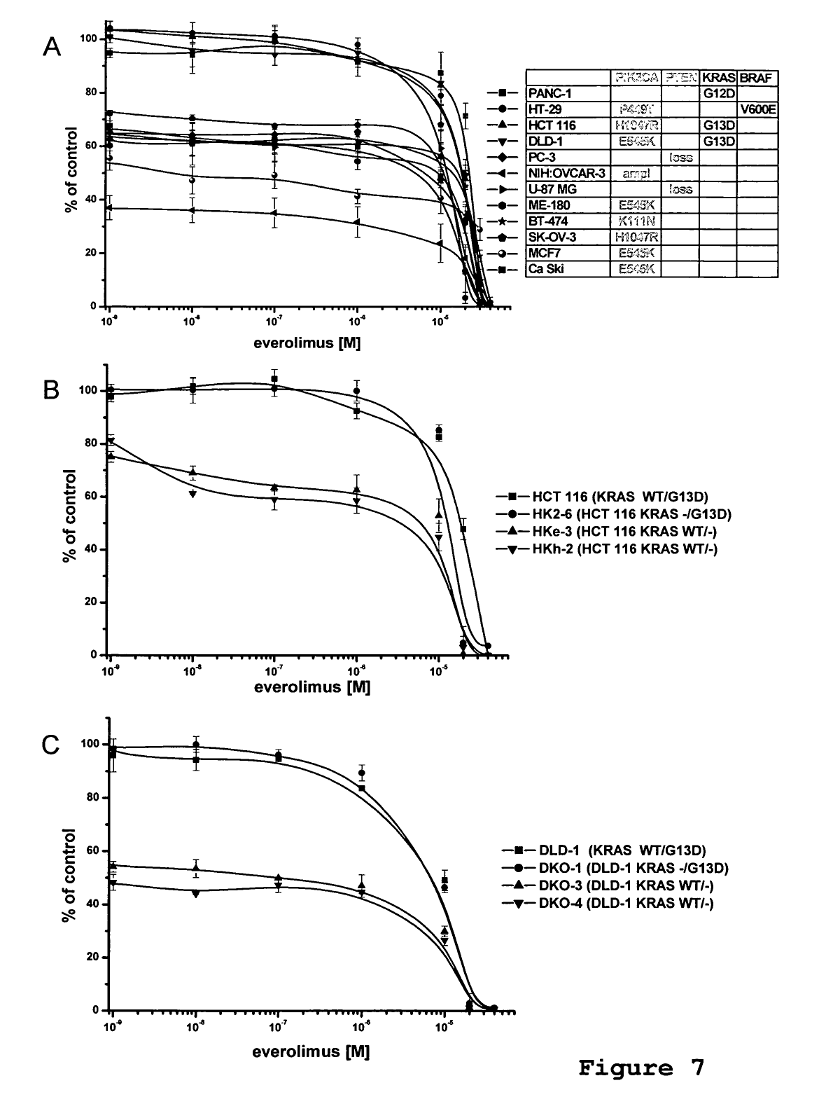

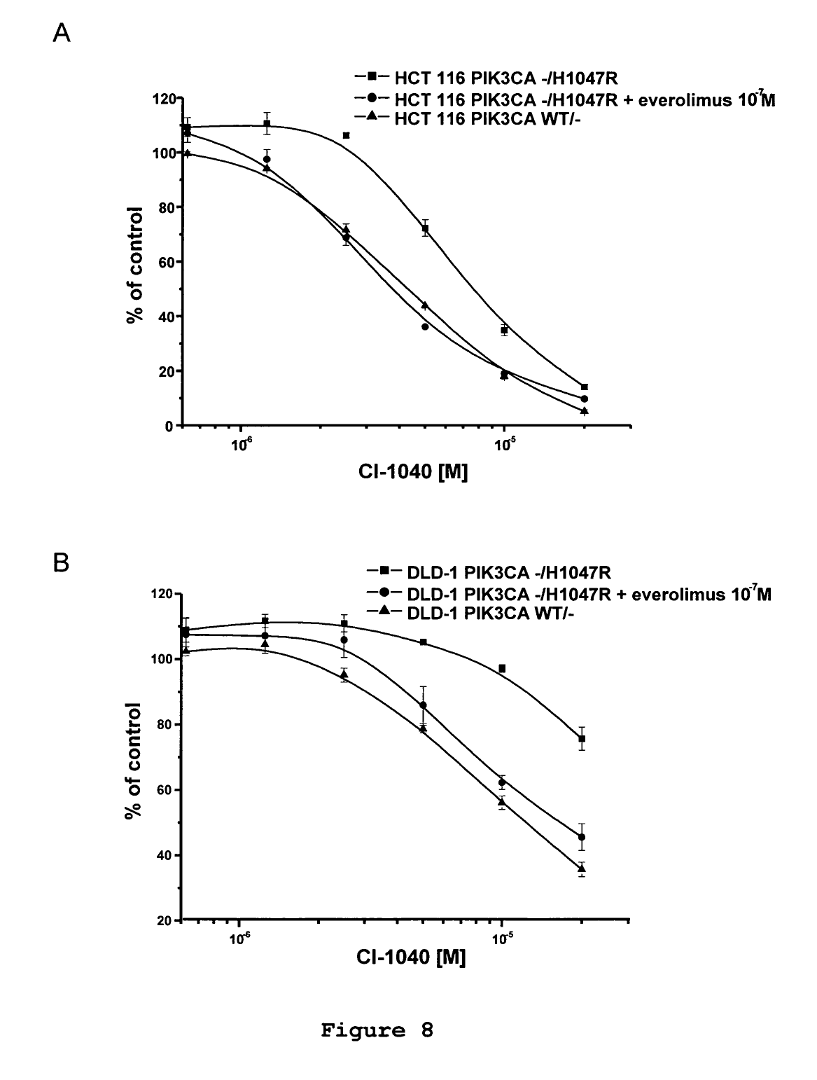

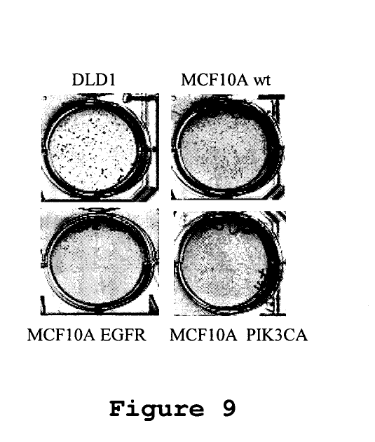

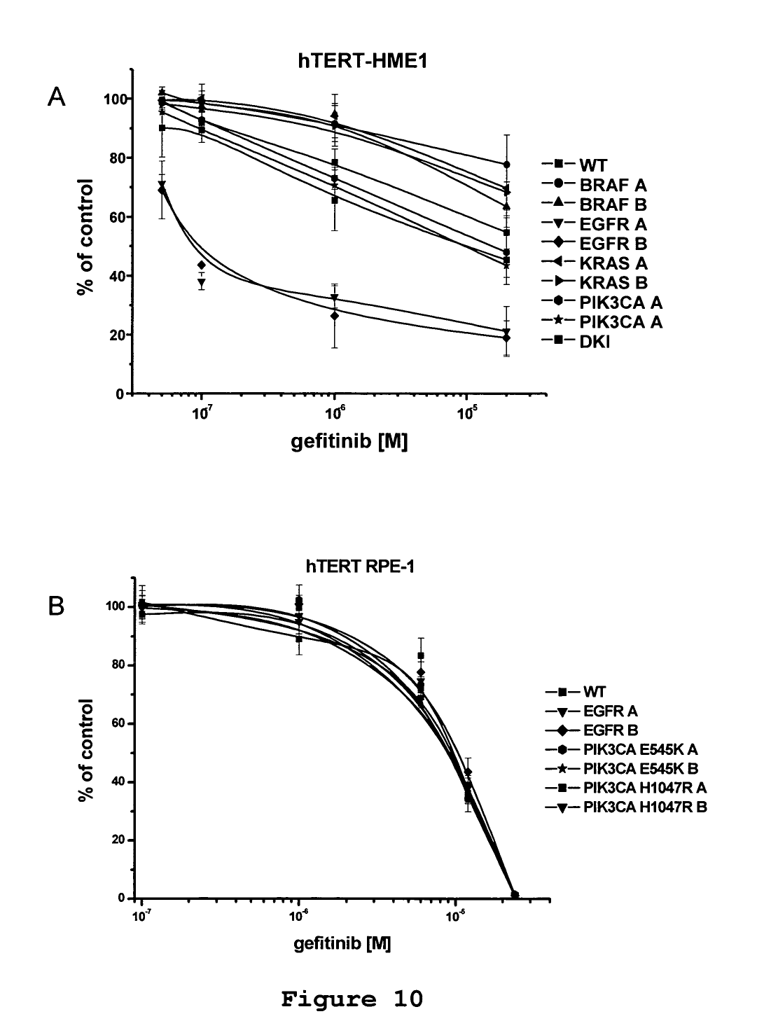

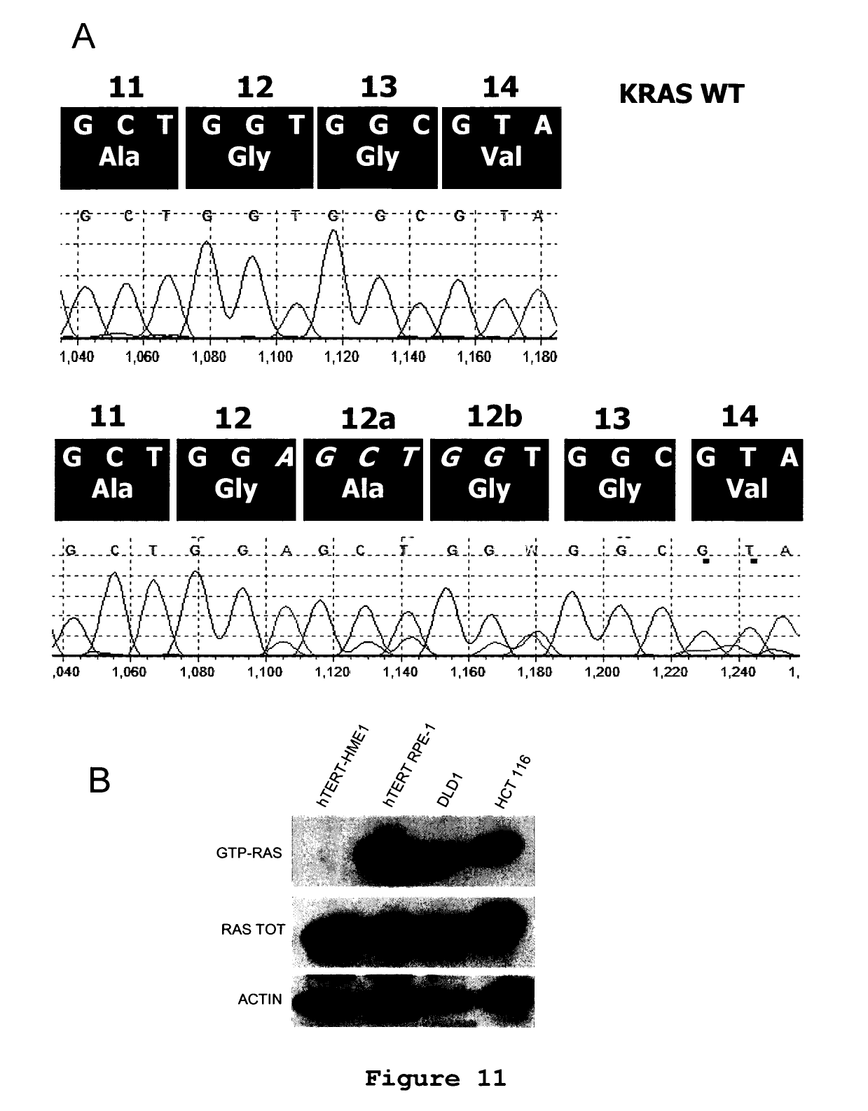

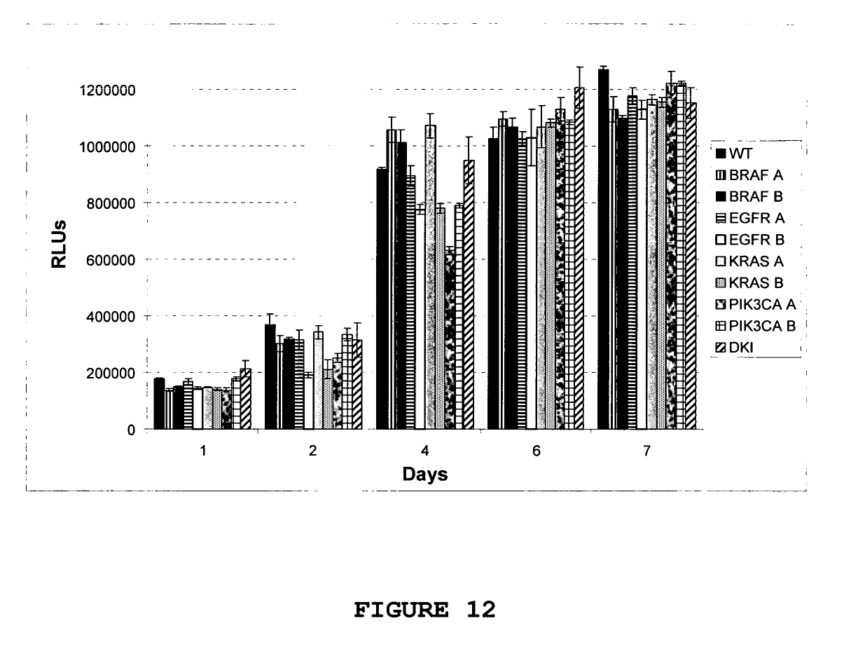

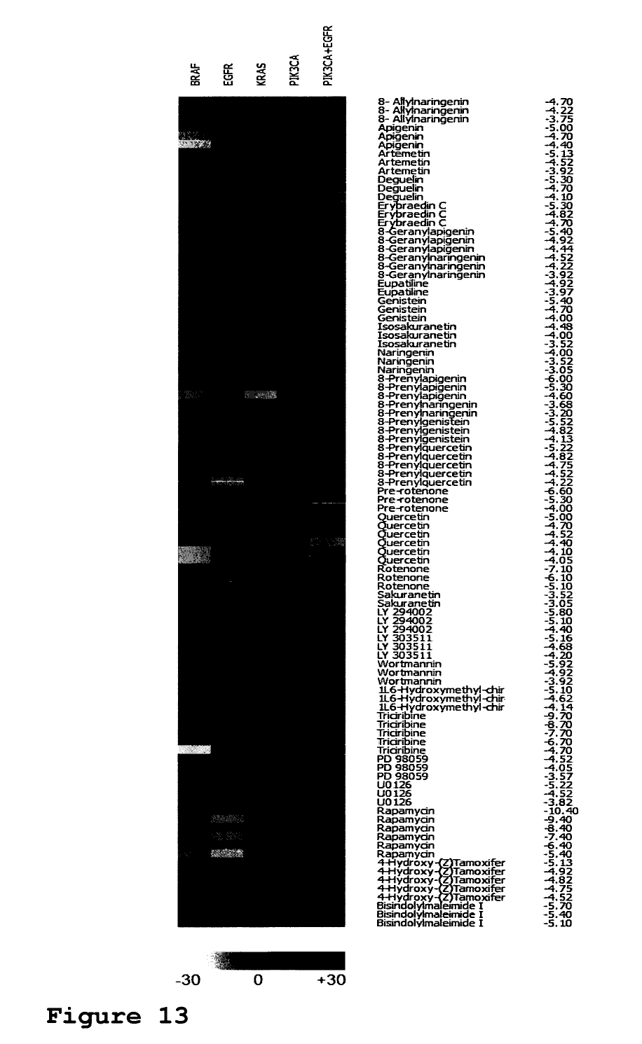

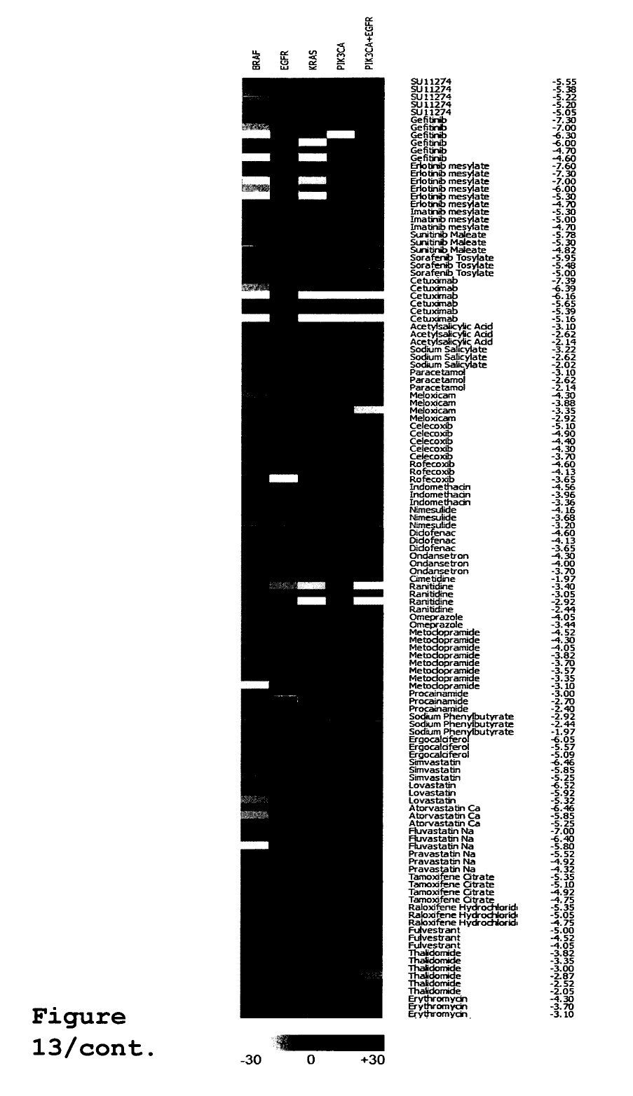

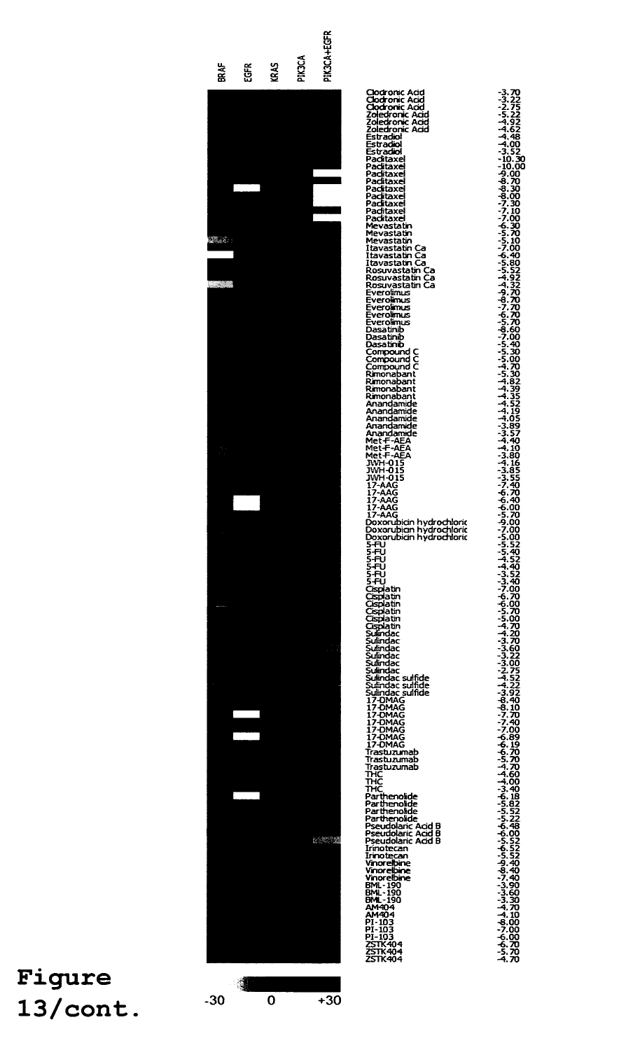

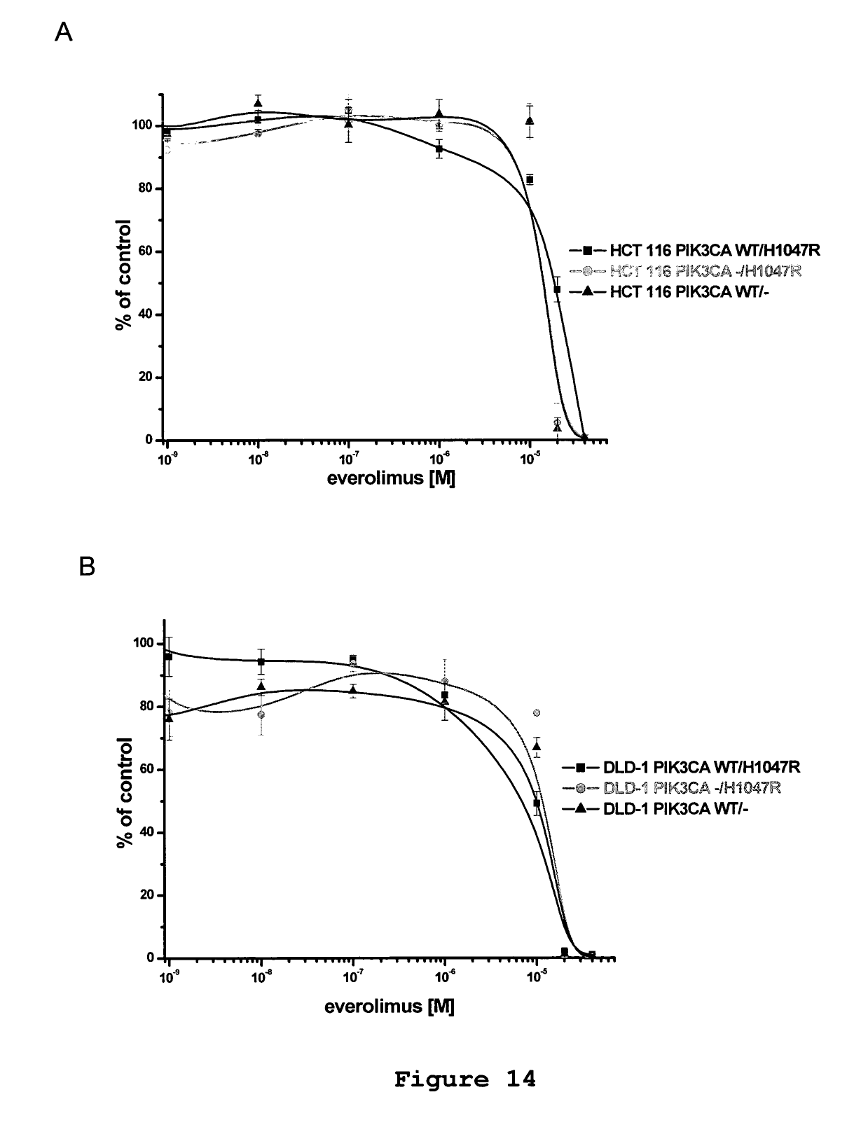

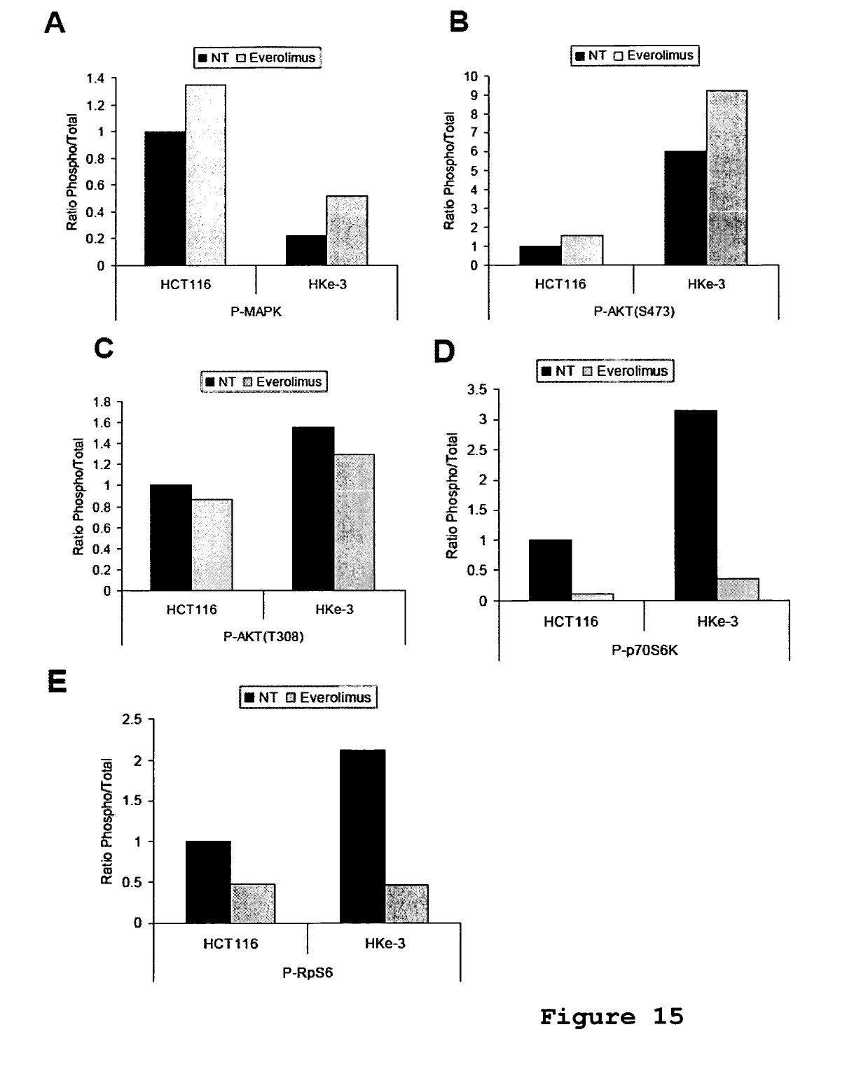

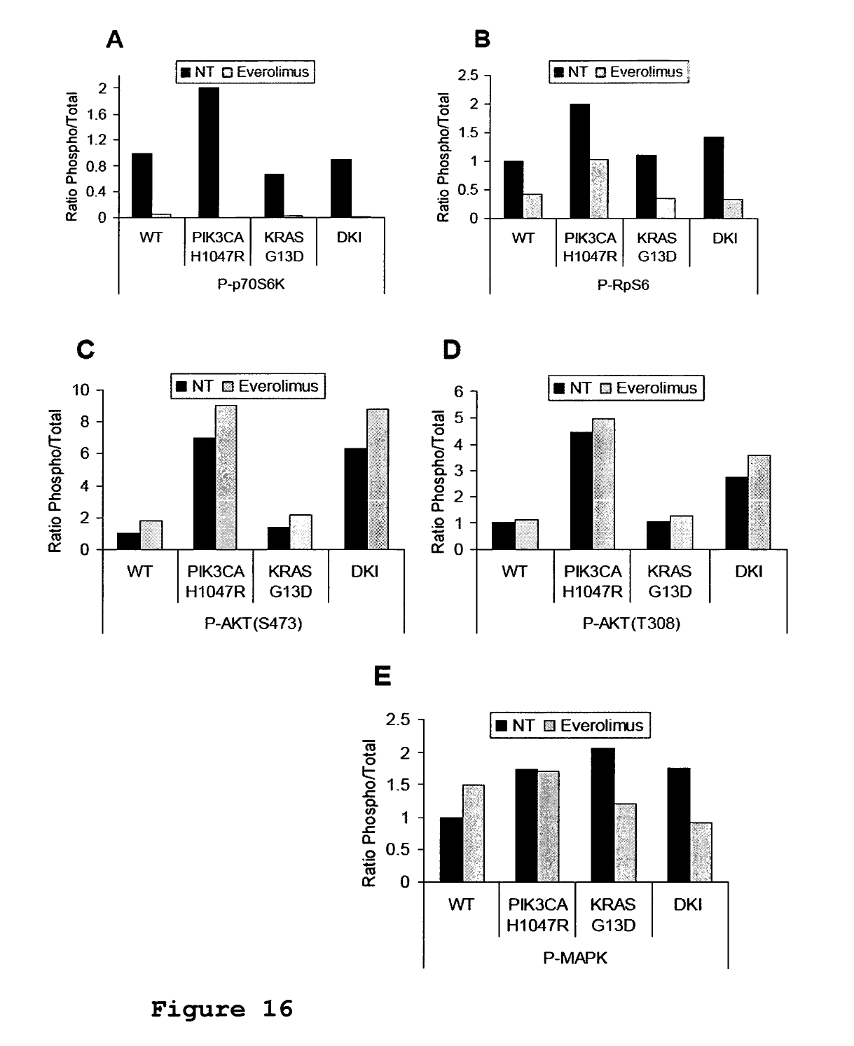

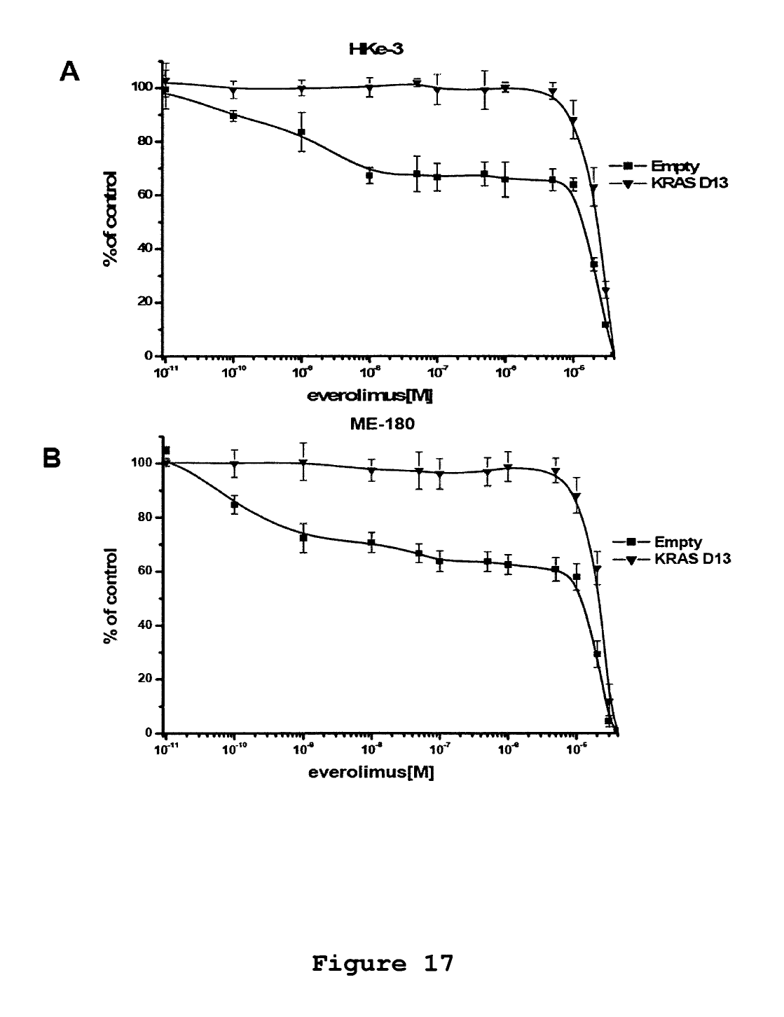

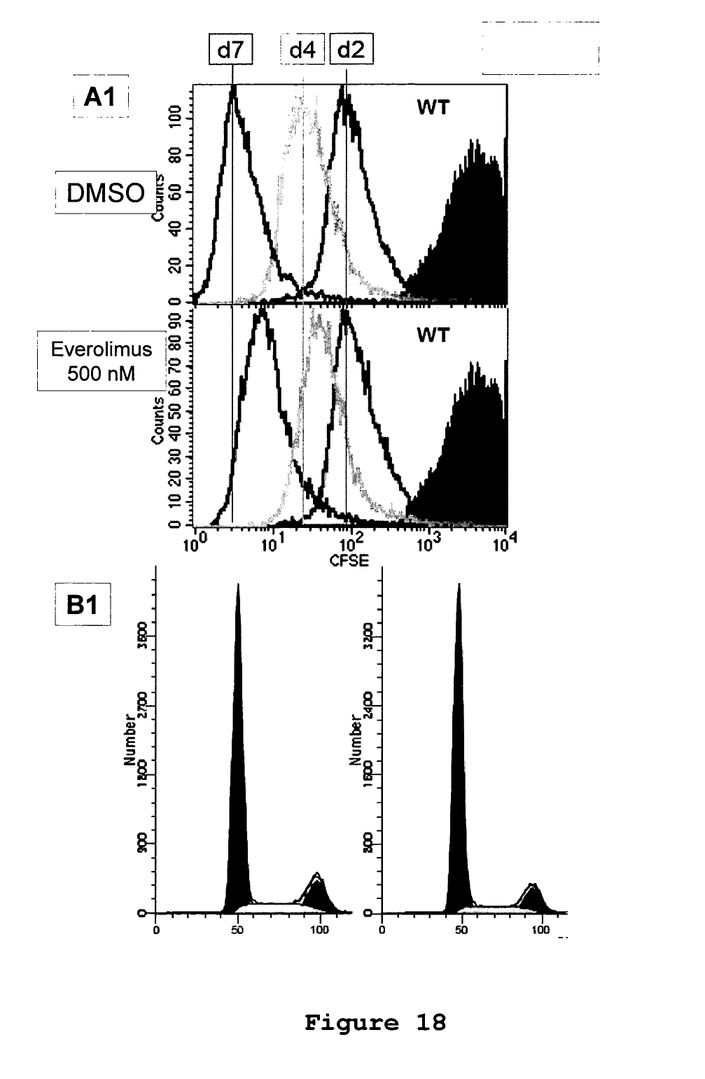

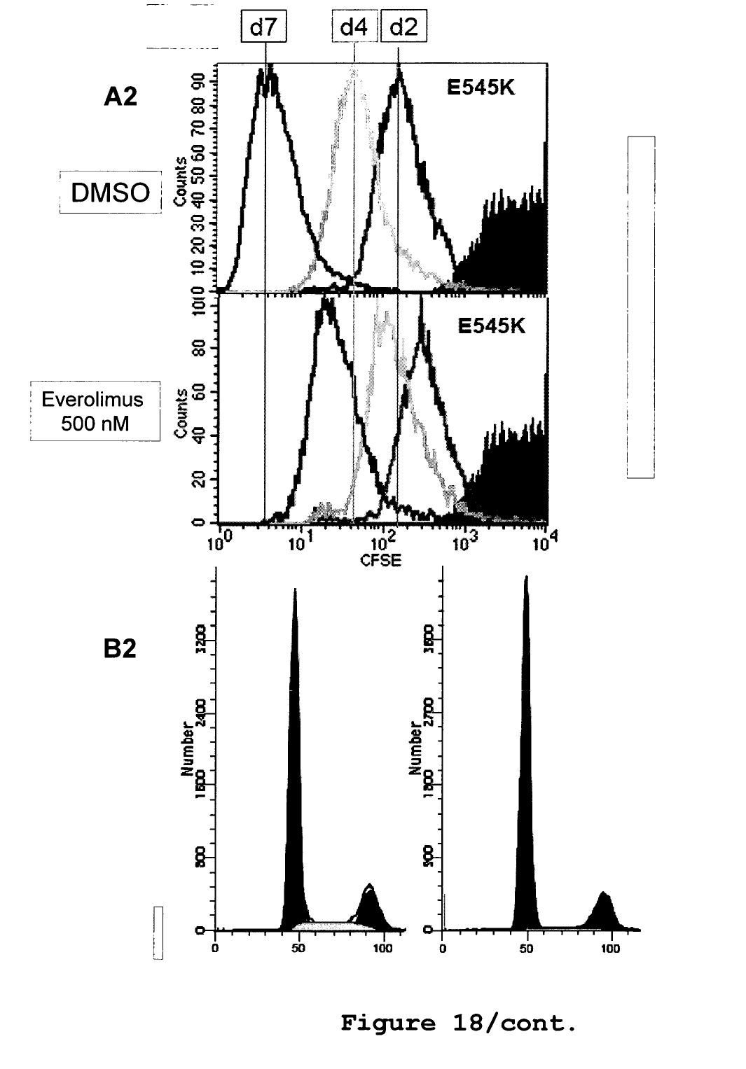

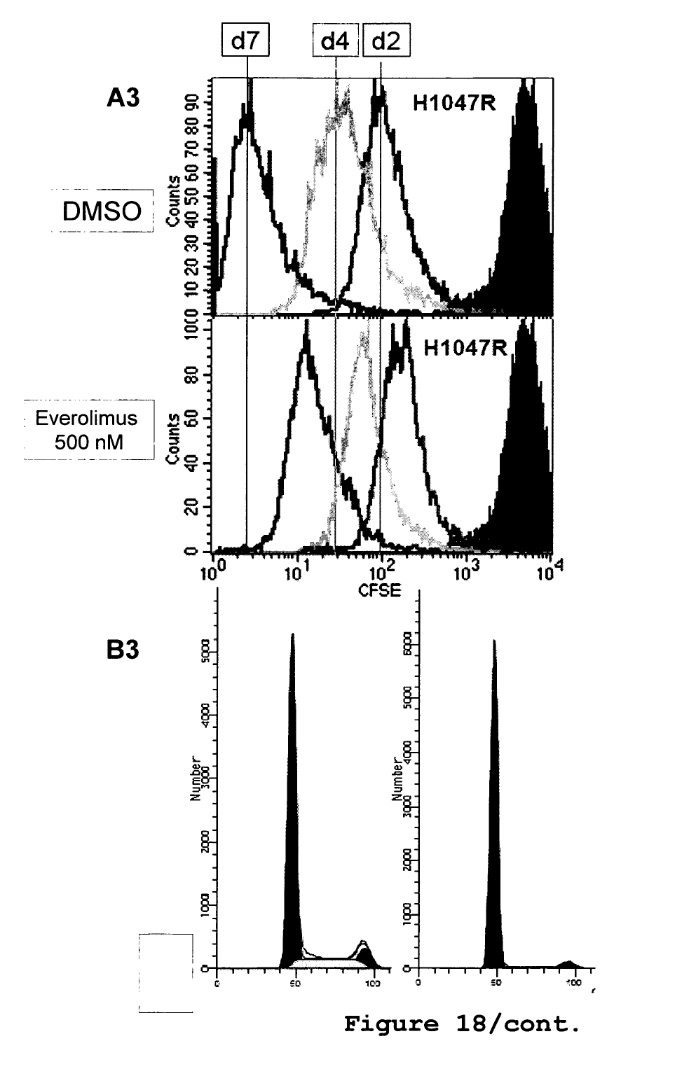

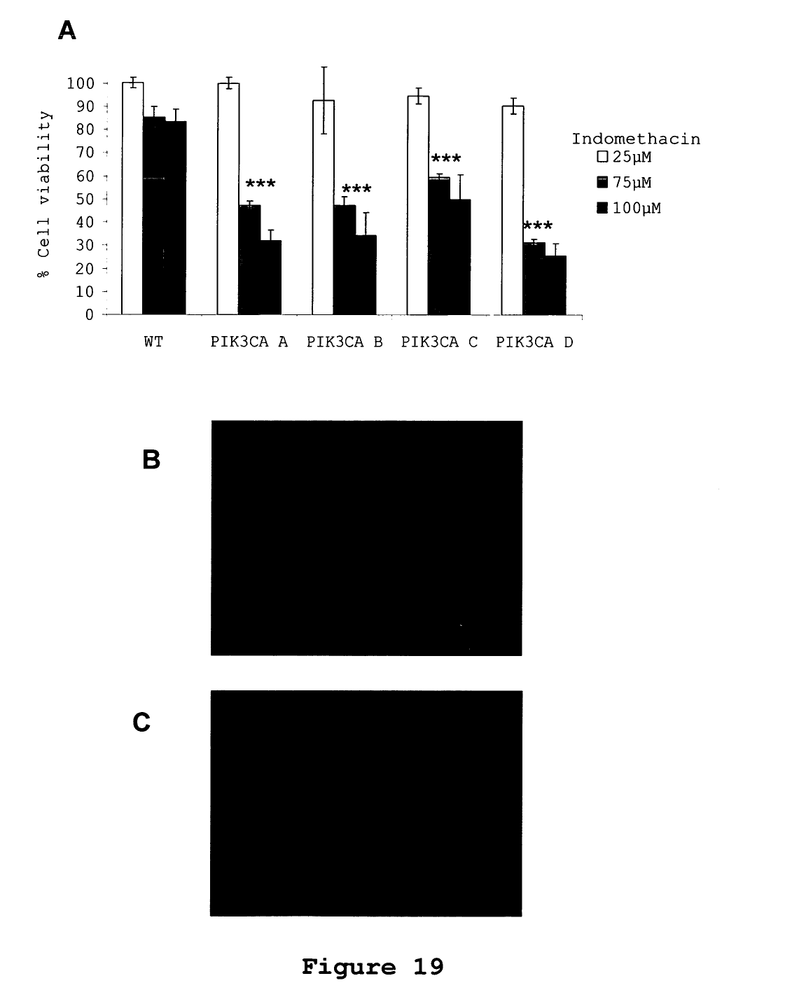

The cancer alleles that were knocked-in in human cells have been previously described to display distinct biochemical and biological properties. Indeed, introduction of oncogenic mutations in the EGFR, KRAS, BRAF and PIK3CA genes in hTERT-HME1 breast cells resulted in activation of the corresponding proteins and triggered specific signaling pathways (FIG. 2). As expected, EGFR KI cells showed striking constitutive (ligand-independent) phosphorylation of EGFR (FIG. 2A). Increased levels of total EGFR protein were also detected; these are likely due to the stabilization of the receptor and reduced degradation imparted by the E746-A750 deletion, as previously shown in lung cancer cells carrying the same allele. Interestingly, DKI cells carrying both the PIK3CA (H1047R) and EGFR (delE746-A750) mutations did not display this phenotype. KRAS, BRAF and PIK3CA mutated cells also displayed allele-specific biochemical features. These included, respectively, PI3K-mediated AKT activation (FIG. 2B), constitutive activation of the KRAS protein as measured by a GTP loading assay (FIG. 2C) and BRAF-initiated activation of the MAPK kinase signaling pathway (FIG. 2D). Similar results were obtained in multiple independent hTERT-HME1 clones of each genotype as well as in the MCF10A and hTERT RPE-1 KI cells carrying the same alleles.

### Transforming Potential of Cancer Alleles Ectopically Expressed or Knocked-in Human Somatic Cells

The in vitro measurable property that more closely correlates with the tumorigenic potential of cancer cells is their ability to grow in anchorage-independent fashion. Accordingly, ectopic expression of the cDNAs corresponding to the four cancer alleles had been previously shown to promote transformation of epithelial cells such as those used in this study.

The oncogenic properties of all KI cells were evaluated by a conventional colony-formation assay in soft agar. The corresponding wild type (WT) cells and the colon cancer cell line HCT 116 were used as negative and positive controls, respectively. EGFR, KRAS and PIK3CA KI hTERT-HME1 cells were unable to grow in soft agar, while BRAF mutated cells gave rise to few small colonies (FIG. 3A). Quantitative assessment of the number of colonies is provided in FIG. 3B. Similarly, no anchorage-independent growth was observed in either MCF10A (FIG. 9) or hTERT RPE-1 cells carrying cancer mutations. Of note, the BRAF mutated cells were not tumorigenic when injected in immunocompromised mice.

These data are in contrast with previous results obtained by overexpression of the corresponding alleles in a number of human cellular models. A direct comparison of the KI versus the ectopic expression methodology was thus performed. To achieve this goal, hTERT-HME1 cells were engineered to express the KRAS and BRAF mutated cDNAs under the control of viral promoters. The results were unequivocal in that hTERT-HME1 cells ectopically expressing any of the corresponding mutated cDNAs readily formed colonies (FIGS. 3A and 3B). In particular, a remarkable difference in the number and size of colonies was observed.

Thus, expression of common cancer alleles under their own promoter is generally not sufficient to transform human epithelial cells.

KI of Cancer Alleles Triggers ‘Oncogene Addiction’ Phenotypes that can be Unveiled by Mutation-Specific Drugs

On this basis, the present KI cell system could offer an unprecedented opportunity to explore the pharmacogenomic properties of cancer alleles, specifically oncogene addiction or resistance to pathway-targeted agents.

As an initial test-case, the ability to induce sensitization in the present isogenic models of EGFR tyrosine kinase inhibitors gefitinib and erlotinib, which are known to preferentially induce apoptosis in cells carrying EGFR somatic mutations, was assessed. Erlotinib preferentially inhibited the growth of hTERT-HME1 and MCF10A KI with the EGFR delE746-A750 allele (FIGS. 4A and 4B). Strikingly, the IC₅₀ values of erlotinib in EGFR mutant cells (0.16±0.06 μM, MCF10A, and 0.25±0.14 μM, hTERT-HME1), were over 10-fold less than those of the corresponding WT cells. Gefitinib showed a similar selectivity pattern (FIG. 10A).

To further dissect this phenomenon DKI cells, containing both EGFR and PIK3CA genetic alterations were treated with gefitinib and erlotinib. Notably, the combination PIK3CA with EGFR abrogates the sensitization seen with the EGFR KI alone (FIG. 4A). This suggests that activation of the PI3K/AKT signaling pathway can circumvent the blockade by EGFR tyrosine kinase inhibitors. These results well agree with recent findings in brain tumors cells that carry similar pathway lesions (EGFR and PTEN alterations) and are resistant to anti EGFR therapies.

Unexpectedly, no selectivity towards EGFR inhibitors was observed in the third cell line (hTERT RPE-1) carrying the EGFR delE746-A750 allele (FIGS. 4C and 10B). The present inventors and others have previously shown that constitutive activation of the RAS/RAF pathway (for example by oncogenic KRAS mutations) can impair the response to drugs targeting EGFR (9, 10). The present inventors therefore considered that a previously unreported activating alteration of the RAS/RAF pathway could be responsible for such lack of effect of erlotinib and gefitinib in hTERT RPE-1 cells. Indeed, mutational analysis of KRAS coding sequence in this line revealed that both the parental and KI cells carried a 6 base-pair insertion in exon 2 of this gene (FIG. 11A). Similar molecular alterations had been previously found in animal and human tumors (11, 12). Biochemical analysis demonstrated that this insertion strongly activates KRAS by permanently switching the corresponding mutated protein into the GTP-bound active state (FIG. 11B). Despite the presence of an activating KRAS mutation, hTERT RPE-1 are not transformed (data not shown), thus further confirming the present finding on the lack of transforming potential of endogenously expressed mutant KRAS alleles. In the present invention hTERT RPE-1 cells have acquired a KRAS gain of function mutation either during the immortalization procedure or during their continuous growth in culture. It is also possible (albeit unlikely) that the tissue of the individual from which the hTERT RPE-1 cells were established was already carrying the corresponding mutated KRAS allele.

Overall, KI of cancer alleles generate cellular models that properly recapitulate the drug response and resistance mechanisms naturally occurring in human tumors.

### Genotype-Specific Clustering of KI Cells by ‘Pharmarray’ Analysis

The striking ‘oncogene addiction’ phenotype demonstrated for EGFR inhibitors in the corresponding KI clones prompted the present inventors to investigate whether similar differential drug responses could be detected in the other KI cells.

To this end a custom library of biologically-active drugs (Table 3) was prepared which comprised:
1. Commonly employed chemotherapeutic agents (e.g. 5-FU, cisplatin)
2. Recently developed kinase inhibitors (e.g. dasatinib)
3. Drugs approved by FDA for a clinical indication other than cancer, but that were previously shown to have an anti-proliferative effect in vitro (e.g. simvastatin)
4. Drugs currently undergoing oncology clinical trials (e.g. everolimus, triciribine)
5. A small collection of natural bioactive compounds (e.g. apigenin, deguelin)
6. A number of ‘pathway specific’ pharmacological tools that were added to the library as controls (e.g. LY294002, PD98059).

Parental and KI cells were seeded in complete growth medium and cell density was assessed by determining cellular ATP content. Under these conditions, no significant differences were observed in the proliferative potential of the KI cells as compared to their normal WT counterpart (FIG. 12). Each compound was then preliminarily tested on WT cells, to determine the concentration referred as the highest no observed effect level (NOEL), the IC₅₀ and the IC₉₀ values. The effects of the drugs on cell viability were measured by the ATP bioluminescent assay. After the initial analysis, all KI clones and parental cells were assayed testing at least three concentrations of each drug (range shown in Table 3) and using a minimum of two clones for each different genotype. The differential activity (ΔKI values, expressed as a percentage of cell growth inhibition) between KI and parental cells was calculated for each compound at a given concentration. The results showed negligible variability among clones carrying the same mutation; therefore, the data obtained from multiple clones for each genotype were averaged. Data analysis details are provided in the Materials and Methods section, and the full set of averaged data of pharmacological responses at each tested drug concentration is provided in Table 6.

Normalized data were further analyzed using data clustering algorithms to better visualize the mutation-specific pharmacological phenotypes in isogenic cell pairs. For this purpose a new software application that the present inventors had previously developed for microarray data clustering and visualization (8) was adopted.

TABLE 6

| | | | | | | | PIK3CA + |
|---|---|---|---|---|---|---|---|
| Compound ID | Compound Name | Log(M) | BRAF | EGFR | KRAS | PIK3CA | EGFR |
| OGC-001 | 8-Allylnaringenin | −4.70 | −1.38 | 0 | 0 | 0 | 0 |
| OGC-001 | 8-Allylnaringenin | −4.22 | 2.08 | 1.68 | 0 | 0 | 4.88 |
| OGC-001 | 8-Allylnaringenin | −3.75 | 0.28 | 0 | 0 | 0 | 0 |
| OGC-002 | Apigenin | −5.00 | 0 | 0 | 0 | 0 | 0 |
| OGC-002 | Apigenin | −4.70 | −2.32 | 0 | 0 | 0 | 0 |
| OGC-002 | Apigenin | −4.40 | −3.18 | 0 | 0 | 0 | 0 |
| OGC-003 | Artemetin | −5.13 | 0 | 0 | 0 | 0 | 1.42 |
| OGC-003 | Artemetin | −4.52 | 0 | 0 | 0 | 0 | 0 |
| OGC-003 | Artemetin | −3.92 | 0 | 0 | 0 | 0 | 0 |
| OGC-004 | Deguelin | −5.30 | 0 | 0 | 4.78 | 0 | 0 |
| OGC-004 | Deguelin | −4.70 | 0 | 0 | 0 | 0 | 6.24 |
| OGC-004 | Deguelin | −4.10 | 0 | 0 | 0 | 0 | 0 |
| OGC-005 | Erybraedin C | −5.30 | 0 | 0 | 0 | 0 | 0 |
| OGC-005 | Erybraedin C | −4.82 | 0 | 0 | 0 | 0 | 0.02 |
| OGC-005 | Erybraedin C | −4.70 | 0 | 0 | 0 | 0 | 0.38 |
| OGC-006 | 8-Geranylapigenin | −5.40 | −0.12 | 0 | 0 | 0 | 2.18 |
| OGC-006 | 8-Geranylapigenin | −4.92 | 2.84 | 2.86 | 0 | 0 | 4.38 |
| OGC-006 | 8-Geranylapigenin | −4.44 | 1.16 | 0 | 0 | 0 | 0 |
| OGC-007 | 8-Geranylnaringenin | −4.52 | −0.84 | 4.16 | 0 | 2.66 | 3.72 |
| OGC-007 | 8-Geranylnaringenin | −4.22 | −0.28 | −0.02 | 0 | 0 | 0.04 |
| OGC-007 | 8-Geranylnaringenin | −3.92 | 0 | 0 | 0 | 0 | 0 |
| OGC-008 | Eupatiline | −4.92 | −0.1 | 0 | 0 | −1.66 | 0 |
| OGC-008 | Eupatiline | −3.97 | 2.9 | 0 | 0 | 0 | 0 |
| OGC-009 | Genistein | −5.40 | 0 | 0 | 0 | 0 | 0 |
| OGC-009 | Genistein | −4.70 | 0 | 3.98 | 0 | 0 | 0 |
| OGC-009 | Genistein | −4.00 | 0 | 0 | 0 | 0 | 0 |
| OGC-010 | Isosakuranetin | −4.48 | 0.7 | 0 | 0 | 0 | 0 |
| OGC-010 | Isosakuranetin | −4.00 | 2.26 | 0 | 0 | 0 | 0 |
| OGC-010 | Isosakuranetin | −3.52 | 0.44 | 0 | 0 | 0 | 0 |
| OGC-011 | Naringenin | −4.00 | −1.5 | 0 | 0 | 0 | 0 |
| OGC-011 | Naringenin | −3.52 | 0 | 0 | 0 | 0 | 0 |
| OGC-011 | Naringenin | −3.05 | −0.08 | 0 | 0 | 0 | 0 |
| OGC-012 | 8-Prenylapigenin | −6.00 | −0.88 | 0 | −0.68 | 0 | 0 |
| OGC-012 | 8-Prenylapigenin | −5.30 | 0.12 | 0 | 0 | 0 | 0.52 |
| OGC-012 | 8-Prenylapigenin | −4.60 | −1.9 | 0 | −2.52 | 0 | −1 |
| OGC-013 | 8-Prenylnaringenin | −3.68 | 0.88 | 1.16 | 0 | 0 | 1.52 |
| OGC-013 | 8-Prenylnaringenin | −3.20 | 0.02 | 0 | 0.02 | 0.02 | 0.02 |
| OGC-014 | 8-Prenylgenistein | −5.52 | −0.38 | 0 | 0 | 0 | 0 |
| OGC-014 | 8-Prenylgenistein | −4.82 | 0.32 | 0 | 0 | 0 | 0 |
| OGC-014 | 8-Prenylgenistein | −4.13 | −1.34 | 0 | 0 | 0 | 0 |
| OGC-015 | 8-Prenylquercetin | −5.22 | 0 | 0.54 | 0 | 0 | 0.84 |
| OGC-015 | 8-Prenylquercetin | −4.82 | 0 | 0 | 0 | 0 | 1.1 |
| OGC-015 | 8-Prenylquercetin | −4.75 | 0 | 0 | 0 | 0 | 0.8 |
| OGC-015 | 8-Prenylquercetin | −4.52 | 0 | 0 | 0 | 0 | 0 |
| OGC-015 | 8-Prenylquercetin | −4.22 | 0 | −2.34 | 0 | 0 | 1.14 |
| OGC-016 | Pre-rotenone | −6.60 | 0 | 0 | 0 | 0 | 0 |
| OGC-016 | Pre-rotenone | −5.30 | 0 | 6.74 | 0 | 0 | 6.64 |
| OGC-016 | Pre-rotenone | −4.00 | 0 | 0 | 0 | 0 | 0 |
| OGC-017 | Quercetin | −5.00 | 0 | 0 | 0 | 0 | 0.62 |
| OGC-017 | Quercetin | −4.70 | 0.64 | 0 | 0 | 0 | 0.24 |
| OGC-017 | Quercetin | −4.52 | 0 | 0 | 0 | 0 | 2.96 |
| OGC-017 | Quercetin | −4.40 | 0 | 0 | 0 | 0 | −1.66 |
| OGC-017 | Quercetin | −4.10 | −2.52 | −1.36 | 0 | 0 | 0.7 |
| OGC-017 | Quercetin | −4.05 | −2.76 | 0 | 0 | 0 | 5.46 |
| OGC-018 | Rotenone | −7.10 | 0 | 0 | 0 | 0 | 0 |
| OGC-018 | Rotenone | −6.10 | 0 | 7.32 | 0 | 0 | 0 |
| OGC-018 | Rotenone | −5.10 | 0 | 0 | 0 | 0 | 0 |
| OGC-019 | Sakuranetin | −3.52 | 2.24 | 0.78 | 0 | 2.4 | 1.46 |
| OGC-019 | Sakuranetin | −3.05 | 0.02 | 0.02 | 0 | 0 | 0.04 |
| OGC-020 | LY 294002 | −5.80 | 0 | 0 | 0 | 0 | 0 |
| OGC-020 | LY 294002 | −5.10 | 0 | 0 | 0 | 3.62 | 0 |
| OGC-020 | LY 294002 | −4.40 | 0 | 0 | 0 | 0 | 0 |
| OGC-021 | LY 303511 | −5.16 | 0 | 0.86 | 0 | 0 | 0.62 |
| OGC-021 | LY 303511 | −4.68 | 0 | 1.1 | 0 | 0 | 1.9 |
| OGC-021 | LY 303511 | −4.20 | 0 | 2.000002 | 0 | 0 | 2.4 |
| OGC-022 | Wortmannin | −5.92 | 0 | 0 | 0 | 0 | 0 |
| OGC-022 | Wortmannin | −4.92 | 0 | 0 | 0 | 0 | 0 |
| OGC-022 | Wortmannin | −3.92 | 0 | 0 | 0 | 0 | 0 |
| OGC-023 | 1L6-Hydroxymethyl- | −5.10 | 0 | 0 | 0 | 0 | 2.4 |
| | chiro-inositol-2-(R)- | | | | | | |
| | 2-O-methyl-3-O- | | | | | | |
| | octadecyl-sn- | | | | | | |
| | glycerocarbonate | | | | | | |
| OGC-023 | 1L6-Hydroxymethyl- | −4.62 | 0 | 0 | 0 | 0 | 3.32 |
| | chiro-inositol-2-(R)- | | | | | | |
| | 2-O-methyl-3-O- | | | | | | |
| | octadecyl-sn- | | | | | | |
| | glycerocarbonate | | | | | | |
| OGC-023 | 1L6-Hydroxymethyl- | −4.14 | 0 | 0 | 0 | 0 | 0 |
| | chiro-inositol-2-(R)- | | | | | | |
| | 2-O-methyl-3-O- | | | | | | |
| | octadecyl-sn- | | | | | | |
| | glycerocarbonate | | | | | | |
| OGC-024 | Triciribine | −9.70 | 0 | 0 | 0 | 0 | 0.14 |
| OGC-024 | Triciribine | −8.70 | 0 | 0 | 0 | 0 | 0 |
| OGC-024 | Triciribine | −7.70 | 0 | 0 | 0 | 0 | 0 |
| OGC-024 | Triciribine | −6.70 | 0 | 5.62 | 0 | 0 | 0 |
| OGC-024 | Triciribine | −4.70 | −3.96 | 9.62 | 0 | 3.54 | 5.92 |
| OGC-025 | PD 98059 | −4.52 | 4.000002 | 0 | 0 | 0 | 0 |
| OGC-025 | PD 98059 | −4.05 | 0 | 3.5 | 0 | 0 | 0 |
| OGC-025 | PD 98059 | −3.57 | 0 | 0 | 0 | 0 | 0 |
| OGC-026 | U0126 | −5.22 | 0 | 0 | 0 | 0 | 0 |
| OGC-026 | U0126 | −4.52 | 0 | 6.76 | 0 | 0 | 0 |
| OGC-026 | U0126 | −3.82 | 0 | 0 | 0 | 0 | 0 |
| OGC-027 | Rapamycin | −10.40 | 0 | 0 | 0 | 0 | 0 |
| OGC-027 | Rapamycin | −9.40 | 0 | −2.38 | 0 | 0 | 0.66 |
| OGC-027 | Rapamycin | −8.40 | 0 | 0 | 0 | 0 | 0 |
| OGC-027 | Rapamycin | −7.40 | 0 | −2.46 | 0 | 0 | 1.6 |
| OGC-027 | Rapamycin | −6.40 | 0 | 0 | 0 | 4.98 | 0 |
| OGC-027 | Rapamycin | −5.40 | −1.84 | −3.1 | 0 | 0 | 1.22 |
| OGC-028 | 4-Hydroxy- | −5.13 | 0 | 0 | 0 | 0 | 3.6 |
| | (Z)Tamoxifen | | | | | | |
| OGC-028 | 4-Hydroxy- | −4.92 | 0 | 3.36 | 0 | 5.66 | 0.92 |
| | (Z)Tamoxifen | | | | | | |
| OGC-028 | 4-Hydroxy- | −4.82 | 0 | 0 | 0 | 0 | 0 |
| | (Z)Tamoxifen | | | | | | |
| OGC-028 | 4-Hydroxy- | −4.75 | 0 | 3.9 | 0 | 0 | 1.52 |
| | (Z)Tamoxifen | | | | | | |
| OGC-028 | 4-Hydroxy- | −4.52 | 0 | 0 | 0 | 0 | 0 |
| | (Z)Tamoxifen | | | | | | |
| OGC-029 | Bisindolylmaleimide I | −5.70 | 0 | 0 | 0 | 0 | 0 |
| OGC-029 | Bisindolylmaleimide I | −5.40 | 0 | 0 | 0 | 0 | 0 |
| OGC-029 | Bisindolylmaleimide I | −5.10 | 0 | 0 | 0 | 0 | 0 |
| OGC-030 | SU11274 | −5.55 | 0 | 0 | 0 | 0 | 0 |
| OGC-030 | SU11274 | −5.38 | 0 | 0 | 0 | 0 | 0 |
| OGC-030 | SU11274 | −5.22 | −1.88 | 0 | 0 | 0 | 3.48 |
| OGC-030 | SU11274 | −5.20 | 0 | 0 | 0 | 0 | 0 |
| OGC-030 | SU11274 | −5.05 | 0 | 0 | 0 | 0.18 | 0.16 |
| OGC-031 | Gefitinib | −7.30 | 0 | 0 | 0 | 0 | 0 |
| OGC-031 | Gefitinib | −7.00 | −2.52 | 0 | 0 | 0 | −0.38 |
| OGC-031 | Gefitinib | −6.30 | −6.26 | 0 | 0 | −4.92 | −0.04 |
| OGC-031 | Gefitinib | −6.00 | 0 | 7.2 | −5.18 | 0 | 0 |
| OGC-031 | Gefitinib | −4.70 | 0 | 5.08 | 0 | 0 | 0 |
| OGC-031 | Gefitinib | −4.60 | −7 | 0 | −7.6 | 0 | 1.46 |
| OGC-032 | Erlotinib mesylate | −7.60 | −0.06 | 0 | 0 | 0.5 | 0.5 |
| OGC-032 | Erlotinib mesylate | −7.30 | 0 | 0 | 0 | 0 | 0 |
| OGC-032 | Erlotinib mesylate | −7.00 | −3.72 | 8 | −3.85 | 0 | 0 |
| OGC-032 | Erlotinib mesylate | −6.00 | −2.66 | 6.88 | 0 | 0 | 0 |
| OGC-032 | Erlotinib mesylate | −5.30 | −6.89 | 5.88 | −5.93 | 0 | 0 |
| OGC-032 | Erlotinib mesylate | −4.70 | 0 | 0 | 0 | 0 | 0 |
| OGC-033 | Imatinib mesylate | −5.30 | 0 | 0 | 0 | 0 | 0 |
| OGC-033 | Imatinib mesylate | −5.00 | 0 | 7.3 | 0 | 9.56 | 0 |
| OGC-033 | Imatinib mesylate | −4.70 | 0 | 0 | 0 | 0 | 0 |
| OGC-034 | Sunitinib Maleate | −5.78 | 0 | 0 | 0 | 0 | 0 |
| OGC-034 | Sunitinib Maleate | −5.30 | 4.42 | 0 | 0 | 0 | 0 |
| OGC-034 | Sunitinib Maleate | −4.82 | 0 | 0 | 0 | 0 | 0 |
| OGC-035 | Sorafenib Tosylate | −5.95 | 0 | 0 | 0 | 0 | 0 |
| OGC-035 | Sorafenib Tosylate | −5.48 | 0 | 0 | 0 | 0 | 6.52 |
| OGC-035 | Sorafenib Tosylate | −5.00 | 0 | 0 | 0 | 0 | 0 |
| OGC-036 | Cetuximab | −7.39 | 0 | 3.08 | 0 | 0 | 0 |
| OGC-036 | Cetuximab | −6.39 | −2.5 | 0 | 0 | 0 | 0 |
| OGC-036 | Cetuximab | −6.16 | −8.6 | 0 | −8.02 | −5.72 | −4.5 |
| OGC-036 | Cetuximab | −5.65 | 0 | 0 | 0 | 0 | 0 |
| OGC-036 | Cetuximab | −5.39 | 0 | 0 | 0 | 0 | 0 |
| OGC-036 | Cetuximab | −5.16 | −8.68 | 0 | −8.74 | −5.7 | −4.72 |
| OGC-037 | Acetylsalicylic Acid | −3.10 | 0 | 0 | 0 | 0 | 0 |
| OGC-037 | Acetylsalicylic Acid | −2.62 | 0 | 0 | 0 | 0 | 0 |
| OGC-037 | Acetylsalicylic Acid | −2.14 | 0 | 0 | 0 | 0 | 0 |
| OGC-038 | Sodium Salicylate | −3.22 | 0 | 0 | 0 | 0 | 0 |
| OGC-038 | Sodium Salicylate | −2.62 | 0 | 0 | 0 | 0 | 0 |
| OGC-038 | Sodium Salicylate | −2.02 | 0 | 0 | 0 | 0 | 0 |
| OGC-040 | Paracetamol | −3.10 | 0 | 0 | 0 | 0 | 0 |
| OGC-040 | Paracetamol | −2.62 | 0 | 0 | 0 | 0 | 0 |
| OGC-040 | Paracetamol | −2.14 | 0 | 0 | 0 | 0 | 0 |
| OGC-041 | Meloxicam | −4.30 | −1.38 | 0 | 0 | 0 | −0.5 |
| OGC-041 | Meloxicam | −3.88 | 0 | 0 | 0 | 0 | 0 |
| OGC-041 | Meloxicam | −3.35 | 0 | 0 | 0 | 0 | −3.58 |
| OGC-041 | Meloxicam | −2.92 | 0 | 0 | 0 | 0 | 0 |
| OGC-042 | Celecoxib | −5.10 | 0.94 | 0 | 0 | 0 | 0 |
| OGC-042 | Celecoxib | −4.90 | 0 | 3.4 | 2.68 | 0 | 0.94 |
| OGC-042 | Celecoxib | −4.40 | 3.42 | 0 | 0 | 0 | 0 |
| OGC-042 | Celecoxib | −4.30 | 0 | 0 | 2.5 | 0 | 1.64 |
| OGC-042 | Celecoxib | −3.70 | 0 | 0 | 0 | 0 | 0 |
| OGC-043 | Rofecoxib | −4.60 | 0 | 0 | 0 | 0 | 0.76 |
| OGC-043 | Rofecoxib | −4.13 | 0 | 0 | 0 | 0 | 7.16 |
| OGC-043 | Rofecoxib | −3.65 | 0 | −6.98 | 0 | 0 | 2.66 |
| OGC-045 | Indomethacin | −4.56 | 0 | 0 | 0 | 0 | 0.96 |
| OGC-045 | Indomethacin | −3.96 | 0 | 0 | 0 | 10.06 | 0 |
| OGC-045 | Indomethacin | −3.36 | 0 | 0 | 0 | 0 | 0 |
| OGC-046 | Nimesulide | −4.16 | 0 | 0 | 0 | 0 | 0 |
| OGC-046 | Nimesulide | −3.68 | 0 | 0 | 0 | 0 | 0 |
| OGC-046 | Nimesulide | −3.20 | 0 | 0 | 0 | 0 | 0 |
| OGC-047 | Diclofenac | −4.60 | 0 | 0 | 0 | 0 | 1.42 |
| OGC-047 | Diclofenac | −4.13 | 0 | 0 | 0 | 0 | 1.48 |
| OGC-047 | Diclofenac | −3.65 | 0 | 1.12 | 0 | 0 | 0 |
| OGC-048 | Ondansetron | −4.30 | 0 | 0 | 0 | 0 | 0 |
| OGC-048 | Ondansetron | −4.00 | 0 | 0 | 0 | 0 | 0 |
| OGC-048 | Ondansetron | −3.70 | 0 | 0 | 0 | 0 | 0 |
| OGC-049 | Cimetidine | −1.97 | 0 | 0 | 0 | 0 | 0 |
| OGC-050 | Ranitidine | −3.40 | 0 | −2.08 | −3.68 | 0 | −5.72 |
| OGC-050 | Ranitidine | −3.05 | −0.88 | −0.38 | 0 | 0.96 | −0.02 |
| OGC-050 | Ranitidine | −2.92 | 0 | 0 | −7.44 | 0 | −5.74 |
| OGC-050 | Ranitidine | −2.44 | 0 | 0 | −0.12 | 0 | −0.04 |
| OGC-051 | Omeprazole | −4.05 | 0 | 1.74 | 0 | 2.98 | 3.8 |
| OGC-051 | Omeprazole | −3.44 | 0 | 0 | 1.46 | 3.28 | 3.88 |
| OGC-052 | Metoclopramide | −4.52 | 0 | 0 | 0 | 0 | 0 |
| OGC-052 | Metoclopramide | −4.30 | 0 | 0 | 0 | 0 | 0 |
| OGC-052 | Metoclopramide | −4.05 | 0 | 0 | 0 | 0 | 0 |
| OGC-052 | Metoclopramide | −3.82 | 0 | 0 | 0 | 0 | 0 |
| OGC-052 | Metoclopramide | −3.70 | −0.66 | 0 | 0 | 0 | 0.2 |
| OGC-052 | Metoclopramide | −3.57 | 0 | 0 | 0 | 0 | 0 |
| OGC-052 | Metoclopramide | −3.35 | 0 | 9.94 | 0 | 0 | 0 |
| OGC-052 | Metoclopramide | −3.10 | −4.48 | 0 | 0 | 0 | 2.8 |
| OGC-053 | Procainamide | −3.00 | 0 | 0 | 0 | 0 | 0 |
| OGC-053 | Procainamide | −2.70 | 0 | 14.12 | 0 | 0 | 0 |
| OGC-053 | Procainamide | −2.40 | 0 | 0 | 0 | 0 | 0 |
| OGC-054 | Sodium Phenylbutyrate | −2.92 | 0 | 0 | 0 | 0 | 0 |
| OGC-054 | Sodium Phenylbutyrate | −2.44 | 0 | 0 | 0 | 0 | 0 |
| OGC-054 | Sodium Phenylbutyrate | −1.97 | 0 | 0 | 0 | 0 | 0 |
| OGC-055 | Ergocalciferol | −6.05 | 0 | 0 | 0 | 0 | 0 |
| OGC-055 | Ergocalciferol | −5.57 | 0 | 0 | 0 | 0 | 0 |
| OGC-055 | Ergocalciferol | −5.09 | 0 | 0 | 0 | 0 | 0 |
| OGC-057 | Simvastatin | −6.46 | 0 | 0 | 0 | 0 | −0.48 |
| OGC-057 | Simvastatin | −5.85 | 0 | 0 | 0 | 0 | 0 |
| OGC-057 | Simvastatin | −5.25 | −1.26 | 0 | 0 | 0 | 0 |
| OGC-058 | Lovastatin | −6.52 | 0 | 0 | 0 | 0 | 0 |
| OGC-058 | Lovastatin | −5.92 | 0 | 4.06 | 0 | 0 | 0 |
| OGC-058 | Lovastatin | −5.32 | −2.18 | 0 | 0 | 0 | 0 |
| OGC-059 | Atorvastatin Ca | −6.46 | 0 | 0 | 0 | 0 | 0 |
| OGC-059 | Atorvastatin Ca | −5.85 | −2.6 | 0 | 0 | 9.44 | 0 |
| OGC-059 | Atorvastatin Ca | −5.25 | 0 | 0 | 0 | 0 | 0 |
| OGC-060 | Fluvastatin Na | −7.00 | 0 | 0 | 0 | 0 | 0 |
| OGC-060 | Fluvastatin Na | −6.40 | 0 | 0 | 0 | 0 | 0 |
| OGC-060 | Fluvastatin Na | −5.80 | −6.52 | 0 | 0 | 0 | 0 |
| OGC-061 | Pravastatin Na | −5.52 | 0 | 0 | 0 | 0 | 0 |
| OGC-061 | Pravastatin Na | −4.92 | 0 | 0 | 0 | 0 | 0 |
| OGC-061 | Pravastatin Na | −4.32 | 0 | 0 | 0 | 0 | 0 |
| OGC-062 | Tamoxifene Citrate | −5.35 | 0 | −0.54 | 0 | 0.9 | −0.92 |
| OGC-062 | Tamoxifene Citrate | −5.10 | 0 | 0 | 0 | 1.42 | 0.96 |
| OGC-062 | Tamoxifene Citrate | −4.92 | 0 | 0 | 0 | 0 | 0.78 |
| OGC-062 | Tamoxifene Citrate | −4.75 | 0 | 0 | 0 | 0 | 0 |
| OGC-063 | Raloxifene | −5.35 | 0 | 0 | 0 | 0 | 0 |
| | Hydrochloride | | | | | | |
| OGC-063 | Raloxifene | −5.05 | 0 | 0 | 0 | 3.2 | 0 |
| | Hydrochloride | | | | | | |
| OGC-063 | Raloxifene | −4.75 | 0 | 0 | 0 | 0 | 3.54 |
| | Hydrochloride | | | | | | |
| OGC-064 | Fulvestrant | −5.00 | 0.34 | 0 | 0 | 0 | 0 |
| OGC-064 | Fulvestrant | −4.52 | −0.6 | 0 | 0 | 0 | 0 |
| OGC-064 | Fulvestrant | −4.05 | 0.18 | 2.36 | 0 | 0 | 0 |
| OGC-065 | Thalidomide | −3.82 | 0 | 0 | 0 | 0 | 0.32 |
| OGC-065 | Thalidomide | −3.35 | 0 | −0.68 | 0 | 0 | −0.8 |
| OGC-065 | Thalidomide | −3.00 | 0 | 0 | 0 | 0 | −0.56 |
| OGC-065 | Thalidomide | −2.87 | 0 | 0 | 0 | 0 | −2.16 |
| OGC-065 | Thalidomide | −2.52 | 0 | 0 | 0 | 0 | 1.02 |
| OGC-065 | Thalidomide | −2.05 | 0 | 0 | 0 | 1.9 | 0 |
| OGC-066 | Erythromycin | −4.30 | 0 | 0 | 0 | 0 | 0 |
| OGC-066 | Erythromycin | −3.70 | 0 | 6.26 | 0 | 0 | 0 |
| OGC-066 | Erythromycin | −3.10 | 0 | 0 | 0 | 0 | 0 |
| OGC-067 | Clodronic Acid | −3.70 | 0 | 0 | 0 | 0 | 0 |
| OGC-067 | Clodronic Acid | −3.22 | 0 | 7.96 | 0 | 0 | 0 |
| OGC-067 | Clodronic Acid | −2.75 | 0 | 2.86 | 0 | 0 | 0 |
| OGC-068 | Zoledronic Acid | −5.22 | 0 | 0 | 0 | 0 | 0 |
| OGC-068 | Zoledronic Acid | −4.92 | 0 | 0 | 0 | 0 | 0 |
| OGC-068 | Zoledronic Acid | −4.62 | 0 | 0 | 0 | 0 | 0 |
| OGC-069 | Estradiol | −4.48 | 0 | 0 | 0 | 0 | 0 |
| OGC-069 | Estradiol | −4.00 | 0 | 0 | 0 | 0 | 0 |
| OGC-069 | Estradiol | −3.52 | 0 | 0 | 0 | 0 | 0 |
| OGC-070 | Paclitaxel | −10.30 | 0 | 0 | 0 | 0 | 0 |
| OGC-070 | Paclitaxel | −10.00 | −1.6 | 0 | 0 | 0 | −0.5 |
| OGC-070 | Paclitaxel | −9.00 | 0 | 0 | 0 | 0 | −6.08 |
| OGC-070 | Paclitaxel | −8.70 | 0 | 0 | 0 | 0 | 0 |
| OGC-070 | Paclitaxel | −8.30 | 0 | −4.98 | 0 | 0 | −6.4 |
| OGC-070 | Paclitaxel | −8.00 | 0 | 0 | 0 | 0 | −6.94 |
| OGC-070 | Paclitaxel | −7.30 | 0 | 0 | 0 | 0 | −7.26 |
| OGC-070 | Paclitaxel | −7.10 | 2.38 | 0 | 0 | 0 | 0 |
| OGC-070 | Paclitaxel | −7.00 | 0 | 0 | 0 | 0 | −6.98 |
| OGC-071 | Mevastatin | −6.30 | 0 | 0 | 0 | 0 | 0 |
| OGC-071 | Mevastatin | −5.70 | 0 | 0 | 0 | 5.3 | 0 |
| OGC-071 | Mevastatin | −5.10 | −2.5 | 0 | 0 | 0 | 0 |
| OGC-072 | Itavastatin Ca | −7.00 | 0 | 0 | 0 | 0 | 0 |
| OGC-072 | Itavastatin Ca | −6.40 | −7.2 | 0 | 0 | 0 | 0 |
| OGC-072 | Itavastatin Ca | −5.80 | 0 | 0 | 0 | 0 | 0 |
| OGC-073 | Rosuvastatin Ca | −5.52 | 0 | 0 | 0 | 0 | 0 |
| OGC-073 | Rosuvastatin Ca | −4.92 | 0 | 0 | 0 | 0 | 0 |
| OGC-073 | Rosuvastatin Ca | −4.32 | −3 | 0 | 0 | 0 | 0 |
| OGC-074 | Everolimus | −9.70 | 0 | 0 | 0 | 0 | 0 |
| OGC-074 | Everolimus | −8.70 | 0 | 0 | 0 | 0 | 0.68 |
| OGC-074 | Everolimus | −7.70 | 0 | 0 | 0 | 6.46 | 0 |
| OGC-074 | Everolimus | −6.70 | 0 | 0 | 0 | 0 | 1.38 |
| OGC-074 | Everolimus | −5.70 | 0 | 0 | 0 | 0 | 0 |
| OGC-075 | Dasatinib | −8.60 | 0 | 0 | 0 | 0 | 0 |
| OGC-075 | Dasatinib | −7.00 | 0 | 0 | 0 | 0 | 0 |
| OGC-075 | Dasatinib | −5.40 | 0 | 1.32 | 0 | 0 | 0 |
| OGC-076 | Compound C | −5.30 | 0 | 0 | 0 | 0 | 0 |
| OGC-076 | Compound C | −5.00 | 0 | 0 | 0 | 0 | 0 |
| OGC-076 | Compound C | −4.70 | 0 | 0 | 0 | 0 | 0 |
| OGC-077 | Rimonabant | −5.30 | 0 | 0 | 0 | 0 | 0 |
| OGC-077 | Rimonabant | −4.82 | 0 | 0 | 0 | 0 | 2.44 |
| OGC-077 | Rimonabant | −4.39 | 0.02 | 0.08 | 0 | 0.08 | 0.06 |
| OGC-077 | Rimonabant | −4.35 | −0.04 | 0 | 0 | 0 | 0 |
| OGC-078 | Anandamide | −4.52 | 0 | 0 | 0 | 0 | 2.06 |
| OGC-078 | Anandamide | −4.19 | 0 | 0 | 0 | 0 | 1.94 |
| OGC-078 | Anandamide | −4.05 | 0 | 0 | 0 | 0 | 0 |
| OGC-078 | Anandamide | −3.89 | 0 | 0 | 0 | 0 | 0 |
| OGC-078 | Anandamide | −3.57 | 0 | 0 | 0 | 0 | 0 |
| OGC-079 | Met-F-AEA | −4.40 | 0.3 | 0 | 0 | 0 | 0 |
| OGC-079 | Met-F-AEA | −4.10 | −1.22 | 0 | 0 | 0 | 0 |
| OGC-079 | Met-F-AEA | −3.80 | −1 | 0 | 0 | 0 | 0 |
| OGC-080 | JWH-015 | −4.16 | 0 | 0 | 0 | 0 | 0 |
| OGC-080 | JWH-015 | −3.85 | 0 | 0 | 0 | 0 | 0 |
| OGC-080 | JWH-015 | −3.55 | 0 | 0 | 0 | 0 | 0 |
| OGC-081 | 17-AAG | −7.40 | 0 | 0 | 0 | 0 | 0 |
| OGC-081 | 17-AAG | −6.70 | 0 | 0 | 0 | 0 | 0 |
| OGC-081 | 17-AAG | −6.40 | 0.42 | −12.18 | 0 | 0 | 2.78 |
| OGC-081 | 17-AAG | −6.00 | 0 | −11.7 | 0 | 0 | 0 |
| OGC-081 | 17-AAG | −5.70 | 0.08 | 0 | 0 | 3 | 1.18 |
| OGC-082 | Doxorubicin | −9.00 | 0 | 0 | 0 | 0 | 0 |
| | hydrochloride | | | | | | |
| OGC-082 | Doxorubicin | −7.00 | 5.94 | 0 | 0 | 0 | 0 |
| | hydrochloride | | | | | | |
| OGC-082 | Doxorubicin | −5.00 | 0 | 0.92 | 0 | 0 | 0 |
| | hydrochloride | | | | | | |
| OGC-083 | 5-FU | −5.52 | −1.02 | 0 | 0 | 0 | 0 |
| OGC-083 | 5-FU | −5.40 | 0 | 0 | 0 | 0 | 0 |
| OGC-083 | 5-FU | −4.52 | 0 | 0 | 0 | 0 | 0 |
| OGC-083 | 5-FU | −4.40 | 0 | 0 | 0 | 0 | 0 |
| OGC-083 | 5-FU | −3.52 | 0 | 0 | 0 | 0 | 0 |
| OGC-083 | 5-FU | −3.40 | 0 | 0 | 0 | 0 | 2.08 |
| OGC-084 | Cisplatin | −7.00 | 0 | 0 | 0 | 0 | −1.32 |
| OGC-084 | Cisplatin | −6.70 | 0 | 0 | 0 | 0 | 0 |
| OGC-084 | Cisplatin | −6.00 | 0 | 0 | 0 | 0 | 0.7 |
| OGC-084 | Cisplatin | −5.70 | 7.62 | 0 | 0 | 0 | 2.16 |
| OGC-084 | Cisplatin | −5.00 | 0 | 4.84 | 0 | 0 | 4.9 |
| OGC-084 | Cisplatin | −4.70 | 3.82 | 0 | 0 | 0 | 0 |
| OGC-085 | Sulindac | −4.20 | 0 | 0 | 0 | 0 | 0 |
| OGC-085 | Sulindac | −3.70 | 0 | 0 | 0 | 0 | 0 |
| OGC-085 | Sulindac | −3.60 | 1.64 | 0 | 0 | 0 | −1.5 |
| OGC-085 | Sulindac | −3.22 | 0 | 0 | 0 | 0 | 0 |
| OGC-085 | Sulindac | −3.00 | 0 | 0 | 0 | 0 | 0 |
| OGC-085 | Sulindac | −2.75 | 0 | 0 | 0 | 0 | 0 |
| OGC-086 | Sulindac sulfide | −4.52 | 0.3 | 1.54 | 0 | 0 | 0 |
| OGC-086 | Sulindac sulfide | −4.22 | −0.28 | 0 | 0 | 0 | 0 |
| OGC-086 | Sulindac sulfide | −3.92 | 1.84 | 0 | 0 | 0 | 0 |
| OGC-087 | 17-DMAG | −8.40 | 0 | 0 | 0 | 0 | 0 |
| OGC-087 | 17-DMAG | −8.10 | 0 | 0 | 0 | 0 | −1.02 |
| OGC-087 | 17-DMAG | −7.70 | 0 | −8.56 | 0 | 0 | 0 |
| OGC-087 | 17-DMAG | −7.40 | 0 | 0 | 0 | 0 | −0.12 |
| OGC-087 | 17-DMAG | −7.00 | 0 | 0 | 0 | 0 | 0 |
| OGC-087 | 17-DMAG | −6.89 | 0 | −12.5 | 0 | 0 | 4.46 |
| OGC-087 | 17-DMAG | −6.19 | 0 | −1.5 | 0 | 0 | 1.4 |
| OGC-088 | Trastuzumab | −6.70 | 0 | 0 | 0 | 0 | 0 |
| OGC-088 | Trastuzumab | −5.70 | 0 | −0.46 | 0 | 0 | 0 |
| OGC-088 | Trastuzumab | −4.70 | 0 | −1.48 | 0 | 0 | 0 |
| OGC-089 | THC | −4.60 | 0.46 | 0 | 0 | 0 | 0 |
| OGC-089 | THC | −4.00 | 0.3 | 0 | 0 | 0 | 0 |
| OGC-089 | THC | −3.40 | 0.88 | 0 | 0 | 0 | 0 |
| OGC-090 | Parthenolide | −6.18 | 0 | −4.66 | 0 | 0 | 2.42 |
| OGC-090 | Parthenolide | −5.82 | 0 | 0 | 0 | 0 | 0 |
| OGC-090 | Parthenolide | −5.52 | 0 | 0 | 0 | 0 | 0 |
| OGC-090 | Parthenolide | −5.22 | 0 | 0 | 0 | 0 | 0 |
| OGC-091 | Pseudolaric Acid B | −6.48 | 0 | 0 | 0 | 0 | 0.54 |
| OGC-091 | Pseudolaric Acid B | −6.00 | 5.04 | 0 | 0 | 0 | 0 |
| OGC-091 | Pseudolaric Acid B | −5.52 | 0 | 0 | 0 | 0 | −2.66 |
| OGC-092 | Irinotecan | −6.52 | 0 | 0 | 0 | 0 | 0 |
| OGC-092 | Irinotecan | −5.52 | 0 | 0 | 0 | 0 | 0 |
| OGC-093 | Vinorelbine | −9.40 | 0 | 0 | 0 | 0 | 0 |
| OGC-093 | Vinorelbine | −8.40 | 0 | 0 | 0 | 0 | 0 |
| OGC-093 | Vinorelbine | −7.40 | 0 | 0 | 0 | 0 | 0 |
| OGC-095 | BML-190 | −3.90 | 0 | 0 | 0 | 0.72 | 0 |
| OGC-095 | BML-190 | −3.60 | 0 | 0 | 0 | 0 | 0 |
| OGC-095 | BML-190 | −3.30 | 0 | 0 | 0 | 0 | 0 |
| OGC-096 | AM404 | −4.70 | −1.68 | 0 | 0 | 0 | 0 |
| OGC-096 | AM404 | −4.10 | −0.12 | 0 | 0 | 0 | 0 |
| OGC-097 | PI-103 | −8.00 | 0 | 0 | 0 | 0 | 0 |
| OGC-097 | PI-103 | −7.00 | 0 | 0 | 0 | 0 | 0 |
| OGC-097 | PI-103 | −6.00 | 0 | 3.9 | 0 | 0 | 0 |
| OGC-098 | ZSTK404 | −6.70 | 0 | 0 | 0 | 0 | 0 |
| OGC-098 | ZSTK404 | −5.70 | 0 | 0 | 0 | 0 | 4.38 |
| OGC-098 | ZSTK404 | −4.70 | 0 | 2.5 | 0 | 0 | 2.18 |

Unclustered ΔKI values are depicted in FIG. 13, while analyzed data (herein defined as ‘pharmarray’) are shown in FIG. 5A for the hTERT-HME1 cell model. Black-colored boxes indicate drugs that—at the indicated concentrations—preferentially inhibited the growth of mutated cells, while white boxes show compounds to which KI cells were more resistant than their WT counterpart does. Grey boxes indicate no significant differences in response between KI and parental cells.

The vast majority of drugs did not show selectivity towards any specific genotype as shown by the predominant black columns. However, the approach successfully identified a set of colored clusters that were cell- and genotype-specific (FIG. 5).

When an unsupervised ‘FuzzySOM’ clustering analysis of the pharmacogenomic data was performed using the pharmarray approach, a clear segregation of the KI cells was readily obtained (FIG. 5A). Specifically, the pharmarray analysis generated genotype-specific trees reflecting the signaling pathways in which the corresponding oncogenic mutations are known to act. These included on one side the cells carrying KRAS and BRAF mutations, on the other side the PIK3CA, EGFR and DKI (PIK3CA+EGFR) clones (FIG. 5A).

The pharmarray analysis presented herein can be more generally applied (analogously to the transcriptome analysis) to interrogate the chemical-genomic properties of normal and tumor cells.

### Profiling Biologically Active Compounds on Cells Carrying Specific Cancer Alleles Unveils Distinct ‘Oncogene Addiction’ or Resistance Phenotypes

To validate its potential, the pharmarray method was initially applied to identify compounds that clustered according to their ability to inhibit EGFR mutated cells selectively. As expected, cetuximab, gefitinib and erlotinib were retrieved, confirming that this strategy can be successfully applied to identify previously validated pharmacogenomic interactions. In addition to gefitinib and erlotinib, the same approach retrieved other less specific but already known EGFR inhibitors, such as genistein and dasatinib (FIG. 5B). Alongside the EGFR sensitive drug cluster, the analysis identified a clearly distinct resistant (white) cluster (FIG. 5E) of drugs to which EGFR mutated cells were less susceptible than their WT counterpart. Among others, this group comprised geldanamycin derivatives (17-DMAG and 17-AAG) and the anti-ERBB2 monoclonal antibody trastuzumab.

Additional ‘resistant’ and ‘sensitive’ genotype-specific clusters were retrieved by the pharmarray approach (FIGS. 5C-5H). For example, an evident ‘green-resistant’cluster of drugs with differential effects on the KRAS and BRAF mutated cells was retrieved by this analysis (FIG. 5H). This group included drugs inhibiting the EGFR, such as gefitinib, erlotinib, and cetuximab, indicating that KRAS, BRAF and PIK3CA mutations could bypass EGFR blockade and protect the cells from the antiproliferative effects observed with such compounds. Moreover, this cluster indicated that BRAF mutated cells are more resistant to several members of the cholesterol-lowering statins, including simvastatin, lovastatin, fluvastatin, mevastatin, itavastatin, and rosuvastatin (FIG. 5H).

Among the other clusters obtained by this analysis two additional prominent ‘black-inhibitory’ clusters of drugs affecting preferentially the PIK3CA+EGFR DKI genotype (FIG. 5C) and the PIK3CA mutated genotype (FIG. 5G) were retrieved by the pharmarray analysis. The latter cluster included known inhibitors of the PI3K pathway, such as LY294002, rapamycin and everolimus. Among the compounds unexpectedly active on PIK3CA mutated cells, the pharmarray retrieved indomethacin (FIG. 5G). Indomethacin is a NSAID widely employed for several forms of arthritis and for closing the patent ductus arteriosus of preterm infants, but is not approved with an oncology indication. An extended analysis confirmed the initial screening results, indicating that this compound acts preferentially on cells carrying the PIK3CA mutation with both anti-proliferative and pro-apoptotic effects (FIG. 19).

Since everolimus is presently undergoing extensive oncology clinical trials, its activity was further characterized on the isogenic cells. Both the hTERT-HME1 PIK3CA KI clones used during the initial screening as well as two additional clones of the same genotype were treated with a wide range of everolimus concentrations and observed a significant antiproliferative effect only in mutated cells. Importantly, as seen in the biochemical and biological experiments presented above, all clones of the same genotype gave comparable results (FIG. 6A). The correlation between the KI of PIK3CA mutations and the sensitivity to everolimus was confirmed also in MCF10A thus excluding that the effect could be cell dependent (FIG. 6B). To assess whether the PIK3CA-everolimus relationship might be mutation-specific and/or could be affected by the occurrence of other tumor related alterations also the SW48 PI3KCA KI were examined. Similar to the results obtained in breast immortalized cells, the introduction of an activating PIK3CA mutation (E545K) in the SW48 background triggered sensitization to everolimus (FIG. 6C).

To shed light on the preferential effect induced by everolimus in PIK3CA KI clones, the present inventors also performed FACS analysis. It has been found that treatment with everolimus of hTERT-HME1 cells resulted in a cytostatic effect that was significantly more pronounced in PIK3CA KI clones compared to their WT counterpart. While vehicle-only treated cells proliferated at a comparable rate, exposure to everolimus for 7 days slowed cell growth in all genotypes, with the effect being particularly evident in PIK3CA H1047R, less pronounced in PIK3CA E545K and only minimal in WT cells (FIG. 18A). Upon treatment, all hTERT-HME1 PIK3CA KI clones accumulated in the G0/G1-phase of the cell cycle (FIG. 18B), and, accordingly, the proportions of cells in the S- and G2/M-phases decreased (Table 7). The data shown in table 7 are the results of hTERT-HME1 cells of the indicated genotype incubated for 48 h with everolimus (500 nM), wherein the effect on cell cycle was analyzed by FACS. Upon treatment, only hTERT-HME1 PIK3CA KI clones significantly accumulated in the G0/G1-phase of the cell cycle. Accordingly, the proportions of cells in the S- and G2/M-phases decreased. Means of at least 4 independent experiments are shown. Significance by paired t test was taken at p<0.01. Apoptosis was almost undetectable and did not vary between vehicle only- or drug-treated cells (FIG. 18B).

TABLE 7

| | |
|---|---|
| | Everolimus |
| DMSO | 500 nM |

| | Mean | SD | Mean | SD | p |
|---|---|---|---|---|---|
| hTERT-HME1 WT | | | | | |
| G1 | 71.6 | 6.6 | 75.7 | 5.9 | .029 |
| G2/M | 14.2 | 2.2 | 14.2 | 4.6 | 0.979 |
| S | 14.7 | 3.9 | 10.1 | 1.7 | 0.047 |
| G2 + S | 28.9 | 5.9 | 24.3 | 6.0 | 0.022 |
| Sub-G1 | 1.0 | 0.3 | 0.9 | 0.4 | 0.543 |
| KI PIK3CA E545K | | | | | |
| G1 | 67.5 | 5.0 | 78.5 | 1.2 | 0.002 |
| G2/M | 17.9 | 4.8 | 12.7 | 3.1 | 0.015 |
| S | 14.6 | 2.4 | 8.8 | 3.5 | 0.002 |
| G2 + S | 32.5 | 5.0 | 21.5 | 1.2 | 0.002 |
| Sub-G1 | 1.0 | 0.3 | 0.9 | 1.2 | 0.811 |
| KI PIK3CA H1047R | | | | | |
| G1 | 73.0 | 3.6 | 88.7 | 4.7 | 0.003 |
| G2/M | 9.6 | 0.6 | 4.9 | 1.2 | 0.003 |
| S | 17.4 | 3.5 | 6.4 | 4.3 | 0.008 |
| G2 + S | 27.0 | 3.6 | 11.3 | 4.7 | 0.003 |
| Sub-G1 | 1.4 | 1.3 | 0.6 | 0.4 | 0.181 |

### The Mutational Status of KRAS and PIK3CA is a Determinant of Response to Everolimus in Human Tumor Cells

The present pharmacogenomic analysis of non-transformed cells carrying cancer alleles point to a relationship between the occurrence of PIK3CA mutations and sensitivity to everolimus. The present inventors next assessed whether and to what extent these findings might be applicable to human cancer cells in which mutations in the PIK3CA pathway naturally occur alongside with additional genetic alterations. To this end a panel of cell lines derived from glioblastoma, breast, ovarian, prostate, endometrial and colorectal carcinomas which are known to carry genetic alterations in PIK3CA or PTEN (FIG. 7) were treated with everolimus. Interestingly, tumor cells could be classified in two main groups based on their response to everolimus (FIG. 7A). Everolimus-resistant cells (such as HT-29, HCT 116 and DLD-1) carried mutations in both PIK3CA and KRAS/BRAF. On the contrary, cells sensitive to this compound displayed PIK3CA pathway alterations but no mutation in the KRAS/BRAF genes (FIG. 7A).

### Genetic Ablation of the KRAS D13 Mutation Restores Sensitivity of Cancer Cells to Everolimus.

The present inventors considered that genetic alterations of the KRAS pathway could represent a major genetic determinant of everolimus resistance in tumor cells carrying PIK3CA oncogenic alleles. To formally test this hypothesis, the present inventors took advantage of HCT 116 cells in which the KRAS D13 mutant allele had been genetically deleted by homologous recombination. Strikingly, it has been found that HCT 116 derivative cells retaining only the KRAS WT allele (named HKh-2 and HKe-3) were sensitive to everolimus, while both the parental and the isogenic cells carrying mutated KRAS were equally resistant to this compound (FIG. 7B). As a further control, we employed HCT 116 cells in which the PIK3CA mutation H1047R had been deleted by targeted homologous recombination. As expected, since all clones retained a mutated KRAS allele, the derivative isogenic cells were non-responsive to everolimus (FIG. 14a).

The effect of everolimus on HCT 116 cells and its derivative KRAS WT clones were also assessed at the biochemical level. As expected, KRAS mutant (HCT 116 parental) cells showed increased MAPK phosphorylation (FIG. 15A). Interestingly, although both cell lines had mutated PIK3CA, KRAS mutant (HCT 116 parental) cells displayed reduced activation of members of the PI3K/AKT/mTOR signaling, including AKT (FIG. 15, B and C), p70S6K (FIG. 5D), RpS6 (FIG. 15E), as compared to the KRAS WT derivatives (HKe-3). This suggests that, in PIK3CA mutated cells, genetic ablation of mutant KRAS determined a compensatory hyper-activation of PI3K/AKT/mTOR signaling. After 30 minutes' treatment with everolimus, phosphorylation of p70S6K was abrogated in both parental and KRAS D13 deleted cells (FIG. 15D), and the levels of activated RpS6 decreased accordingly (FIG. 15E). In addition, drug treated HKe-3 cells showed higher level of MAPK phosphorylation compared to the parental counterpart carrying the KRAS oncogenic allele (FIG. 15A).

### Knock-in or Ectopic Expression of Mutated KRAS Abrogates Everolimus' Sensitivity of Cells Carrying PIK3CA Mutations.

To further explore the role of mutant KRAS on everolimus' response, the present inventors recapitulated the genetic milieu of the HCT116 colorectal cancer cells in hTERT-HME1 cells, by introducing via homologous recombination both KRAS G13D and PIK3CA H1047R alleles in their genome. This approach generated double-KI (DKI) cells, in which each mutation is expressed under the corresponding gene's own promoter. When exposed to everolimus, these double mutant cells displayed a cell cycle response comparable to that observed in the parental WT population The data shown in table 8 are the results of hTERT-HME1 cells of the indicated genotype incubated for 48 h with everolimus (500 nM), wherein cell nuclei were stained with propidium iodide and the effect on cell cycle was analyzed by FACS. Means of at least 4 independent experiments are shown. Significance by paired t test was taken at p<0.01.

TABLE 8

| | |
|---|---|
| | Everolimus |
| DMSO | 500 nM |

| | Mean | SD | Mean | SD | p |
|---|---|---|---|---|---|
| hTERT-HME1 WT | | | | | |
| G1 | 71.6 | 6.6 | 75.7 | 5.9 | 0.029 |
| G2/M | 14.2 | 2.2 | 14.2 | 4.6 | 0.979 |
| S | 14.7 | 3.9 | 10.1 | 1.7 | 0.047 |
| KI PIK3CA H1047R | | | | | |
| G1 | 73.0 | 3.6 | 88.7 | 4.7 | 0.003 |
| G2/M | 9.6 | 0.6 | 4.9 | 1.2 | 0.003 |
| S | 17.4 | 3.5 | 6.4 | 4.3 | 0.008 |
| KI KRAS G13D | | | | | |
| G1 | 73.3 | 6.2 | 75.7 | 9.0 | 0.325 |
| G2/M | 13.6 | 1.4 | 13.1 | 3.7 | 0.668 |
| S | 13.0 | 5.4 | 11.2 | 5.5 | 0.204 |
| DKI KRAS G13D + | | | | | |
| PIK3CA H1047R | | | | | |
| G1 | 70.9 | 2.8 | 77.5 | 8.6 | 0.216 |
| G2/M | 10.8 | 1.3 | 10.5 | 3.9 | 0.869 |
| S | 18.2 | 1.6 | 12.0 | 4.7 | 0.077 |

As expected, p70S6K phosphorylation was abrogated by drug treatment (FIG. 16A) and this was accompanied by a decrease of activated phospho-RpS6 levels (FIG. 16B). An increase of phospho-AKT was also present in all genotypes upon drug exposure (FIG. 16, C and D). Notably, after everolimus treatment, levels of phospho-MAPK resulted essentially unchanged in WT cells and in PIK3CA H1047R mutated cells, while were decreased in KRAS G13D mutated cells (FIG. 16E).

Next, it has been assessed whether these results could be confirmed in cancers cells. The present inventors transduced HCT116-derivative clones that had only the KRAS WT allele (HKe-3), and the endometrial cancer cell line ME-180 (carrying PIK3CA E545K mutant and KRAS WT) with a lentiviral vector encoding for KRAS G13D cDNA.

(Re-)Introduction of mutated KRAS resulted in decreased response to the antiproliferative effects of everolimus when compared to cells transduced with a control vector (FIG. 17, A and B).

Combinatorial Pharmacological Suppression of mTOR and MEK is Synergistic in Human Colorectal Cancer Cells Carrying KRAS and PIK3CA Oncogenic Mutations

The above observations indicate that, in cancer cells carrying PIK3CA mutations, genetic targeting of the KRAS oncogenic pathway results in everolimus sensitivity. The present inventors set out to verify whether these results could be recapitulated by combinatorial pharmacological modulation of both KRAS and PIK3CA in cancer cells. The development of specific mutated kras inhibitors has so far remained elusive; the present inventors therefore employed a compound, CI-1040 (also known as PD 184352) that inhibits one of kras immediate downstream signaling effectors, MEK. According to working hypothesis, the present inventors predicted that HCT 116 and DLD-1 isogenic cells retaining only the WT PIK3CA (PIK3CA WT/−) allele would be more sensitive to CI-1040 than those carrying mutated PIK3CA (PIK3CA-/H1047R). Experimental verification indeed showed that the MEK inhibitor affects to a greater extent PIK3CA WT/− cancer cells than their isogenic mutant pairs (FIGS. 8A and 8B). Notably, and further confirming the present findings, treatment of PIK3CA mutant cells with a combination of CI-1040 and a single-fixed clinically relevant concentration of everolimus (10⁻⁷ M) had effects comparable to those achievable by the MEK inhibitor alone in PIK3CA WT/− cells (FIGS. 8A and 8B).

The nature of CI-1040/everolimus pharmacological interaction was further evaluated using the combination index method (13). Over a wide range of concentrations, the combination of these two compounds synergistically inhibited the proliferation of both HCT 116 and DLD-1 colorectal cancer cells resulting in combination indices (CI₅₀) of 0.67 and 0.40, respectively.

The combined genetic and pharmacological analysis indicate that combinatorial targeting of both the KRAS/MEK/MAPK and PIK3CA/AKT/mTOR pathways could result in synergistic antiproliferative activity in cancer cells displaying concomitant mutations in KRAS and PIK3CA.

Naturally, while the principle of the invention remains the same, the details of construction and the embodiments may widely vary with respect to what has been described and illustrated purely by way of example, without departing from the scope of the present invention.

### REFERENCES

1. Arena, S., Isella, C., Martini, M., de Marco, A., Medico, E., and Bardelli, A. 2007. Knock-in of Oncogenic Kras Does Not Transform Mouse Somatic Cells But Triggers a Transcriptional Response that Classifies Human Cancers. Cancer Res 67:8468-8476.
2. Hua, V. Y., Wang, W. K., and Duesberg, P. H. 1997. Dominant transformation by mutated human ras genes in vitro requires more than 100 times higher expression than is observed in cancers. Proc Natl Acad Sci USA 94:9614-9619.
3. Verhaegen, M., Bauer, J. A., Martin de la Vega, C., Wang, G., Wolter, K. G., Brenner, J. C., Nikolovska-Coleska, Z., Bengtson, A., Nair, R., Elder, J. T., et al. 2006. A novel BH3 mimetic reveals a mitogen-activated protein kinase-dependent mechanism of melanoma cell death controlled by p53 and reactive oxygen species. Cancer Res 66:11348-11359.
4. Greenman, C., Stephens, P., Smith, R., Dalgliesh, G. L., Hunter, C., Bignell, G., Davies, H., Teague, J., Butler, A., Stevens, C., et al. 2007. Patterns of somatic mutation in human cancer genomes. Nature 446:153-158.
5. Sjoblom, T., Jones, S., Wood, L. D., Parsons, D. W., Lin, J., Barber, T. D., Mandelker, D., Leary, R. J., Ptak, J., Silliman, N., et al. 2006. The consensus coding sequences of human breast and colorectal cancers. Science 314:268-274.
6. Samuels, Y., Diaz, L. A., Jr., Schmidt-Kittler, O., Cummins, J. M., Delong, L., Cheong, I., Rago, C., Huso, D. L., Lengauer, C., Kinzler, K. W., et al. 2005. Mutant PIK3CA promotes cell growth and invasion of human cancer cells. Cancer Cell 7:561-573.
7. Arena, S., Pisacane, A., Mazzone, M., Comoglio, P. M., and Bardelli, A. 2007. Genetic targeting of the kinase activity of the Met receptor in cancer cells. Proc Natl Acad Sci USA 104:11412-11417.
8. Fu, L., and Medico, E. 2007. FLAME, a novel fuzzy clustering method for the analysis of DNA microarray data. BMC bioinformatics 8:3.
9. Benvenuti, S., Sartore-Bianchi, A., Di Nicolantonio, F., Zanon, C., Moroni, M., Veronese, S., Siena, S., and Bardelli, A. 2007. Oncogenic activation of the RAS/RAF signaling pathway impairs the response of metastatic colorectal cancers to anti-epidermal growth factor receptor antibody therapies. Cancer Res 67:2643-2648.
10. Pao, W., Wang, T. Y., Riely, G. J., Miller, V. A., Pan, Q., Ladanyi, M., Zakowski, M. F., Heelan, R. T., Kris, M. G., and Varmus, H. E. 2005. KRAS mutations and primary resistance of lung adenocarcinomas to gefitinib or erlotinib. PLoS Med 2:e17.
11. Bollag, G., Adler, F., elMasry, N., McCabe, P. C., Conner, E., Jr., Thompson, P., McCormick, F., and Shannon, K. 1996. Biochemical characterization of a novel KRAS insertion mutation from a human leukemia. J Biol Chem 271:32491-32494.
12. Higinbotham, K. G., Rice, J. M., Buzard, G. S., and Perantoni, A. O. 1994. Activation of the K-ras gene by insertion mutations in chemically induced rat renal mesenchymal tumors. Oncogene 9:2455-2459.
13. Chou, T. C., and Talalay, P. 1984. Quantitative analysis of dose-effect relationships: the combined effects of multiple drugs or enzyme inhibitors. Adv Enzyme Regul 22:27-55.

1. An isogenic human cell line, comprising at least one mutated cancer allele, in that said at least one mutated cancer allele is under the control of an endogenous promoter of said cell line, said endogenous promoter being corresponding to the wild-type cancer allele promoter, and in that said cancer allele is selected from the group consisting of BRAF, EGFR, PIK3CA, PTEN, CTNNB1, c-KIT, c-MET, EPHA3, Erbb2, AKT1, FGFR2, MSH6, ABL1, STAT1, STAT4, RET, AKT3, TEK, VAV3, ALK, LYN, NOTCH, IDH1, ROR1, FLT3, ALK, SRC, BCL9, RPS6KA2, PDPK1, NTRK3, NTRK2, AKT3, KDR, MKK4, FBWX7, MEK1, OBSCN, TECTA, MLL3, NRAS, HRAS, TP53, APC, RbI, CDKN2A (p16), BRCA1, BRCA2, PTCH1, VHL, SMAD4, PER1, MEN1, NF1, NF2, ATM, and PTPRD.

2. The isogenic human cell line according to claim 1, wherein said cell line carries at least two mutated cancer alleles, wherein said at least two cancer alleles are selected from the group consisting of BRAF, EGFR, PIK3CA, PTEN, CTNNB1, c-KIT, c-MET, EPHA3, Erbb2, AKT1, FGFR2, MSH6, ABL1, STAT1, STAT4, RET, AKT3, TEK, VAV3, ALK, LYN, NOTCH, IDH1, ROR1, FLT3, ALK, SRC, BCL9, RPS6KA2, PDPK1, NTRK3, NTRK2, AKT3, KDR, MKK4, FBWX7, MEK1, OBSCN, TECTA, MLL3, NRAS, HRAS, TP53, APC, RbI, CDKN2A (plβ), BRCA1, BRCA2, PTCH1, VHL, SMAD4, PER1, MEN1, NF1, NF2, ATM, PTPRD, and KRAS.

3. The isogenic human cell line according to claim 1, wherein said at least one mutated cancer allele is selected from the group consisting of the mutated cancer alleles listed in Table 2a.

4. The isogenic human cell line according to claim 1, wherein said human cell line is selected from the group consisting of among MCFlOA, hTERT-HME1, HTERT-RPE-I, HCT 116, DLD-I, SW48, NuLi, CuFi, CHON-001, CHON-002, BJ-5ta, hTERT-HME1 (ME16C), hTERT RPE-I, hTERT-HPNE, NeHepLxHT, T HESCs, RWPE-I, RWPE-2, WPE-stem, WPE-int, WPE1-NA22, WPE1-NB14, WPE1-NBI1, WPE1-NB26, RWPE2-W99, WPMY-I, WPE1-NB26-64, WPE1-NB26-65, HBE4-E6/E7 [NBE4-E6/E7], JVM-13, MeT-5A, BBM, BZR, BEAS-2B, MCF 1OA, MCF 1OF, MCF-10-2A, B-3, HBE4-E6/E7-C1, HK-2, CHON-001, CHON-002, HS-5, PWR-IE, THLE-3, HCE-2 [50. B1], 46BR. IN, BRISTOL 8, AGLCL, C211, GM1899A, GS-109-V-63, GS-109-V-34, H9, HFFF2, HFL1, HG261, HH-8, HL, Hs 68, Hs 888Lu, HsI. Tes, IM 9, MRC-5 pd19, MRC-5 pd25, MRC-5 pd30, MRC-5 pd30, MRC-5 SV1 TG1, MRC-5 SV1 TG2, MRC-5 SV2, MRC-7, MRC-9, MT-2, PNT1A, PNT1A (SERUM FREE), PNT2, PNT2 (SERUM FREE), SVCT, SVCT-MI2, TK6, TK6TGR, TOU (TOU 1-2), WI 26 VA4, WI 38, WI 38VA13 Subline 2RA, WiDr, WIL2 NS, WIL2.NS.6TG, WILCL, OVCAR-5, OVCAR-4, OVCAR-3, NCI-H522, NCI-H460, NCI-H322M, NCI-H23, NCI-H226, NCI/ADR-RES, MOLT-4, MDA-N, MDA-MB-435, MDA-MB-231, MCF7, Malme-3M, M14, LOXIMV1, KM12, K-562, IGROV1, HT-29, Hs 578T, HOP-92, HOP-62, HL-60, HCT-15, HCT-116, HCC-2998, EKVX, DU-145, COLO-205, CCRF-CEM, CAKI-I, BT-549, ACHN, A549, A498, and 786-0 cell lines.

5. The isogenic human cell line according to claim 1, wherein the mutated BRAF cancer allele carries the mutation V600E as shown in SEQ ID No.: 3.

6. The isogenic human cell line according to claim 1, wherein the mutated EGFR cancer allele carries the mutation delE746-A750 as shown in SEQ ID No.: 5.

7. The isogenic human cell line according to claim 1, wherein the mutated PIK3CA cancer allele carries the mutations E545K and H1047R as shown in SEQ ID No.: 1 and 2, respectively.

8. The isogenic human cell line according to claim 1, wherein the mutated CTNNB1 cancer allele carries the mutation T41A as shown in SEQ ID No.: 6.

9. The isogenic human cell line according to claim 1, wherein the mutated PTEN cancer allele carries the mutation R130* as shown in SEQ ID No.: 7.

10. The isogenic human cell line according to claim 1, wherein said cell line carries at least one detectable marker.

11. The isogenic human cell line according to claim 10, wherein said at least one marker is selected among a fluorescent, radioactive, luminescent, phosphorescent marker.

12. The isogenic human cell line according to claim 1, wherein said cell line carries at least one knocked-out or inactivated tumor suppressor gene.

13. The isogenic human cell line according to claim 12, wherein said at least one tumor suppressor gene is selected from the group consisting of PTEN, TP53, APC, p21, RbI, BUB1, BRCA1, BRCA2, PTCH, VHL, SMAD4, PER1, TSC2, CDKN2A, DCC, MEN-I, NF1, ATM, PTPRD, LRP1B and NF2.

14. A method of using an isogenic human cell line according to claim 1 for generating xenografts apt to induce tumor growth in a non-human laboratory animal model.

15. A method of using of an isogenic human cell line according to claim 1 for producing non-human transgenic laboratory animals susceptible to develop a tumor, said tumor carrying at least one mutated cancer allele, said cancer allele being selected from the group consisting of BRAF, EGFR, PIK3CA, PTEN, CTNNB1, c-KIT, c-MET, EPHA3, Erbb2, AKT1, FGFR2, MSH6, ABL1, STAT1, STAT4, RET, AKT3, TEK, VAV3, ALK, LYN, NOTCH, IDH1, ROR1, FLT3, ALK, SRC, BCL9, RPS6KA2, PDPK1, NTRK3, NTRK2, AKT3, KDR, MKK4, FBWX7, MEK1, OBSCN, TECTA, MLL3, NRAS, HRAS, TP53, APC, RbI, CDKN2A (plβ), BRCA1, BRCA2, PTCH1, VHL, SMAD4, PER1, MEN1, NF1, NF2, ATM, PTPRD, and KRAS.

16. The method of claim 15, wherein said non-human transgenic laboratory animals carrying a tumor are used for determining the sensitivity/resistance of said tumor to a pharmacological agent administered to said transgenic animals.

17. An in vitro method for determining sensitivity/resistance of a patient suffering from a tumor to a pharmacological agent, characterized in that said process comprises: a) identifying at least one mutated cancer allele in a tissue affected by a tumor of said patient; b) providing an isogenic human cell line representative of said tissue, said cell line comprising at least said mutated cancer allele, wherein said cancer allele is under the control of an endogenous promoter of said cell line, said endogenous promoter being corresponding to the wild-type cancer allele promoter; c) putting in contact said isogenic cell line with said pharmacological agent; d) determining a variation of proliferation, cytotoxicity and/or apoptosis of said isogenic cell line in presence of said pharmacological agent; said variation of proliferation, cytotoxicity and/or apoptosis being indicative of said sensitivity/resistance of said patient to said pharmacological agent.

18. The method of claim 17, wherein said process further comprises; b1) providing a wild-type isogenic human cell line representative of said tissue, being said wild-type isogenic human cell line free of said mutated cancer allele; c1) putting in contact said wild-type isogenic cell line with said pharmacological agent; d1) determining a variation of proliferation, cytotoxicity and/or apoptosis of said wild-type isogenic cell line in presence of said pharmacological agent.

19. The method of claim 17, wherein said sensitivity/resistance is evaluated as the relative variation of proliferation, apoptosis and/or cytotoxicity between said isogenic human cell line comprising said at least mutated cancer allele and said wild-type isogenic human cell line.

20. The method of claim 17, wherein said pharmacological agent is selected from the group consisting of chemotherapeutic agents, tyrosine kinase inhibitors, antiproliferative agents, antiemetics, antacids, H2 antagonists, proton pump inhibitors, laxatives, anti-obesity drugs, antidiabetics, vitamins, dietary minerals, antithrombotics, antihemorrhagics, antianginals, antihypertensives, diuretics, vasolidators, beta blockers, calcium channel blockers, rennin-angiotensin system drugs, antihyperlipidemics (statins, fibrates, bile acid sequestrants), antipsoriatic, sex hormones, hormonal contraceptives, fertility agents, SERMs, hypothalamic-pituitary hormones, corticosteroids (glucocorticoids, mineralocorticoids), thyroid hormones/antithyroid agents, antibiotics, antifungals, antimycobacterial, antivirals, vaccines, antiparasitic (antiprotozoals, anthelmintics), immunomodulators (immunostimulators, immunosuppressants), anabolic steroids, anti-inflammatories (NSAID), antirheumatics, corticosteroids, muscle relaxants, bisphosphonate, anesthetics, analgesics, antimigraines, anticonvulsants, mood stabilizers, antiparkinson drug, psycholeptic (anxiolytics, antipsychotics, hypnotics/sedatives), psychoanaleptic (antidepressants, stimulants/psychostimulants), decongestants, bronchodilators, and H1 antagonists.

21. The method of claim 17, wherein said isogenic human cell line is selected from the group consisting of MCFlOA, hTERT-HME1, HTERT-RPE-I, HCT 116, DLD-I, SW48, NuLi, CuFi, CHON-001, CHON-002, BJ-5ta, hTERT-HME1 (ME16C), hTERT RPE-I, hTERT-HPNE, NeHepLxHT, T HESCs, RWPE-I, RWPE-2, WPE-stem, WPE-int, WPE1-NA22, WPE1-NB14, WPE1-NBIl, WPE1-NB26, RWPE2-W99, WPMY-I, WPE1-NB26-64, WPE1-NB26-65, HBE4-E6/E7 [NBE4-E6/E7], JVM-13, MeT-5A, BBM, BZR, BEAS-2B, MCF 1OA, MCF 1OF, MCF-10-2A, B-3, HBE4-E6/E7-C1, HK-2, CHON-001, CHON-002, HS-5, PWR-IE, THLE-3, HCE-2 [50. B1], 46BR. IN, BRISTOL 8, AGLCL, C211, GM1899A, GS-109-V-63, GS-109-V-34, H9, HFFF2, HFL1, HG261, HH-8, HL, Hs 68, Hs 888Lu, HsI. Tes, IM 9, MRC-5 pd19, MRC-5 pd25, MRC-5 pd30, MRC-5 pd30, MRC-5 SV1 TG1, MRC-5 SV1 TG2, MRC-5 SV2, MRC-7, MRC-9, MT-2, PNT1A, PNT1A (SERUM FREE), PNT2, PNT2 (SERUM FREE), SVCT, SVCT-MI2, TK6, TK6TGR, TOU (TOU 1-2), WI 26 VA4, WI 38, WI 38VA13 Subline 2RA, WiDr, WIL2 NS, WIL2.NS.6TG, WILCL, OVCAR-5, OVCAR-4, OVCAR-3, NCI-H522, NCI-H460, NCI-H322M, NCI-H23, NCI-H226, NCI/ADR-RES, MOLT-4, MDA-N, MDA-MB-435, MDA-MB-231, MCF7, Malme-3M, M14, LOXIMV1, KM12, K-562, IGROVT, HT-29, Hs 578T, HOP-92, HOP-62, HL-60, HCT-15, HCT-116, HCC-2998, EKVX, DU-145, COLO-205, CCRF-CEM, CAKI-I, BT-549, ACHN, A549, A498, and 786-0 cell lines.

22. The method of claim 17, wherein said cancer allele is selected from the group consisting of BRAF, EGFR, PIK3CA, PTEN, CTNNB1, c-KIT, c-MET, EPHA3, Erbb2, AKT1, FGFR2, MSH6, ABL1, STAT1, STAT4, RET, AKT3, TEK, VAV3, ALK, LYN, NOTCH, IDH1, ROR1, FLT3, ALK, SRC, BCL9, RPS6KA2, PDPK1, NTRK3, NTRK2, AKT3, KDR, MKK4, FBWX7, MEK1, OBSCN, TECTA, MLL3, NRAS, HRAS, TP53, APC, RbI, CDKN2A (p16), BRCA1, BRCA2, PTCH1, VHL, SMAD4, PER1, MEND, NF1, NF2, ATM, PTPRD, and KRAS.

23. The method of claim 17, wherein said at least one mutated cancer allele is selected from the group consisting of the mutated cancer alleles listed in Table 2a.

24. A cell bank comprising a plurality of isogenic human cell lines, wherein said cell lines comprise at least one mutated cancer allele, wherein said at least one mutated cancer allele is under the control of an endogenous promoter of said cell line, said endogenous promoter being corresponding to the wild-type cancer allele promoter.

25. The cell bank of claim 24, wherein said cell line carries at least two mutated cancer alleles.

26. The cell bank of claim 24, wherein said cell lines are selected from the group consisting of MCFlOA, hTERT-HME1, HTERT-RPE-I, HCT 116, DLD-I, SW48, NuLi, CuFi, CHON-001, CHON-002, BJ-5ta, hTERT-HME1 (ME16C), hTERT RPE-I, hTERT-HPNE, NeHepLxHT, T HESCs, RWPE-I, RWPE-2, WPE-stem, WPE-int, WPE1-NA22, WPE1-NB14, WPE1-NBIl, WPE1-NB26, RWPE2-W99, WPMY-1, WPE1-NB26-64, WPE1-NB26-65, HBE4-E6/E7 [NBE4-E6/E7], JVM-13, MeT-5A, BBM, BZR, BEAS-2B, MCF 10A, MCF 10F, MCF-10-2A, B-3, HBE4-E6/E7-C1, HK-2, CHON-001, CHON-002, HS-5, PWR-IE, THLE-3, HCE-2 [50. B1], 46BR. IN, BRISTOL 8, AGLCL, C211, GM1899A, GS-109-V-63, GS-109-V-34, H9, HFFF2, HFL1, HG261, HH-8, HL, Hs 68, Hs 888Lu, HsI. Tes, IM 9, MRC-5 pd19, MRC-5 pd25, MRC-5 pd30, MRC-5 pd30, MRC-5 SV1 TG1, MRC-5 SV1 TG2, MRC-5 SV2, MRC-7, MRC-9, MT-2, PNT1A, PNT1A (SERUM FREE), PNT2, PNT2 (SERUM FREE), SVCT, SVCT-MI2, TK6, TK6TGR, TOU (TOU 1-2), WI 26 VA4, WI 38, WI 38VA13 Subline 2RA, WiDr, WIL2 NS, WIL2.NS.6TG, WILCL, OVCAR-5, OVCAR-4, OVCAR-3, NCI-H522, NCI-H460, NCI-H322M, NCI-H23, NCI-H226, NCI/ADR-RES, MOLT-4, MDA-N, MDA-MB-435, MDA-MB-231, MCF7, Malme-3M, M14, LOXIMV1, KM12, K-562, IGROV1, HT-29, Hs 578T, HOP-92, HOP-62, HL-60, HCT-15, HCT-116, HCC-2998, EKVX, DU-145, COLO-205, CCRF-CEM, CAKI-I, BT-549, ACHN, A549, A498, and 786-0 cell lines.

27. The cell bank of claim 24, wherein said at least one cancer allele is selected among BRAF, EGFR, PIK3CA, PTEN, CTNNB1, C-KIT, C-MET, EPHA3, Erbb2, AKT1, FGFR2, MSH6, ABL1, STAT1, STAT4, RET, AKT3, TEK, VAV3, ALK, LYN, NOTCH, IDH1, ROR1, FLT3, ALK, SRC, BCL9, RPS6KA2, PDPK1, NTRK3, NTRK2, AKT3, KDR, MKK4, FBWX7, MEK1, OBSCN, TECTA, MLL3, NRAS, HRAS, TP53, APC, RbI, CDKN2A (p16), BRCA1, BRCA2, PTCH1, VHL, SMAD4, PER1, MEN1, NF1, NF2, ATM, PTPRD, and KRAS.

28. The cell bank of claim 23, wherein said at least one mutated cancer allele is selected from the group consisting of the mutated cancer alleles listed in Table 2a.

29. Everolimus for use in the treatment of a patient suffering from a tumor, wherein said tumor carries a mutated PIK3CA cancer allele and is free of a KRAS mutated cancer allele.

30. Indomethacin for use as a medicament in the treatment of a patient suffering from a tumor.
